(19) United States
(12) Patent Application Publication
(10) Pub. No.: US 2020/0405868 A1
(43) Pub. Date: Dec. 31, 2020

(54) GEL-FORMING POLYPEPTIDES

(71) Applicant: Adepthera LLC, Palo Alto, CA (US)

(72) Inventor: Sheau Yu Teddy Hsu, Menlo Park, CA (US)

(21) Appl. No.: 16/970,894

(22) PCT Filed: Mar. 14, 2019
(86) PCT No.: PCT/US19/22275

§ 371 Date: Aug. 18, 2020

Related U.S. Application Data
(60) Provisional application No. 62/642,593, filed on Mar. 13, 2018.

(51) Int. Cl. A61K 47/42; A61K 9/00; A61K 9/06

(52) U.S. Cl. CPC A61K 47/42; A61K 45/06; A61K 9/06; A61K 9/0024

(57) ABSTRACT

Stable aqueous gel or semisolid gel pharmaceutical composition comprising a gel-forming and water-soluble peptide are optionally combined with an appropriate excipient and a therapeutic agent. The pharmaceutical composition forms a gel depot following administration to an individual, where the gel nanostructure releases the peptide or the encapsulated therapeutic agent(s) over a extended period of time. The gel-forming compounds can be formulated in aqueous, suspension, or solid formulations, comprising 0.01% to 99% of a gel-forming polypeptide by weight with relation to the total weight of the formulation. These formulations are useful in drug delivery and as implantable drug depot for long term delivery of therapeutic agents, antigens, or cells. Also provided are methods of producing a gel-forming compound, through the modification of a chemical compound with a gel-forming enhancing motif.

### CROSS REFERENCE

This application claims benefit of U.S. Provisional Patent Application No. 62/643,593, filed Mar. 15, 2018 which application is incorporated herein by reference in its entirety.

### FIELD

Provided herein are compositions and methods for sustained delivery of therapeutic agents, provided as stable aqueous formulations of gel-forming polypeptide compounds that deliver sustained release of therapeutic agents.

### BACKGROUND

Among common therapeutic modalities, polypeptides represent a middle group between ‘small molecule’ drugs and larger injectable biologics and nucleic acid-based therapeutics. Therapeutic peptides and proteins are generally administered parenterally, e.g., by subcutaneous injection. An advantage of synthetic peptides is that they can incorporate non-natural amino acids to reduce instability and proteolytic cleavage liabilities as well as provide better manufacturing reproducibility.

As therapeutics, most peptides suffer from drawbacks, such as the need for frequent injections due to short half-lives resulting from sensitivity to proteolysis and rapid renal clearance. Many therapeutic treatments require either the continuous or repeated administration over an extended period of time, leading to both discomfort and inconvenience to patients. Therefore, sustained-release formulation or half-life extension technologies have been developed to deliver polypeptide and protein therapeutics over a prolonged period of time. The use of sustained release formulations also allows the delivery of therapeutics to selected tissues or organs, in order to minimize systemic adverse effects.

Techniques employing slow-release nano-structures can improve pharmacokinetic and pharmadynamic properties of therapeutics following delivery into the body, or following topical treatments. This technique can provide sustained regulation of cellular signaling and reduce the peak-to-trough effect of therapeutic agents (e.g. small molecule drugs, peptides, hormones, proteins, nucleic acids, cells, or pro-drugs). Biocompatible nanoscale drug carriers have the potential to provide substantial improvement of drug delivery, and to improve efficacy and reduce systemic side effects. For example, oil suspension and crystal particle suspension have been used extensively for sustained delivery of small molecule drugs. In these procedures, the oil and solid particle are used to create a barrier for the dispersion of soluble therapeutics.

Polymeric gels have been used for the delivery of small molecules and polypeptides, including gel-forming peptides, such as lanreotide. These gel-forming peptides form polymers and aqueous gel at high concentrations, in contrast to the majority of polypeptides, which are either present as aqueous solution or becomes an insoluble precipitate at high concentrations.

Biocompatible polymeric peptides and hydrogels have a wide variety of applications in biotechnology and medicine, especially in the controlled delivery and release of drugs and therapeutics, in addition to functioning as supports in tissue engineering (see, for example, (U.S. Pat. Nos. 5,034,229; 5,057,318; and 5,110,596). Hydrogels can contain networks of monomers that interact to give self-supporting, hydrogen-bonded nanostructures, or that form through hydrophobic interactions and Van der Waals force. For example, polymeric microcapsules and matrixes with polylactic polymers have been used for the delivery of small molecule, peptide, and protein drugs (e.g., Kent, et al., U.S. Pat. No. 4,675,189). Hydrogel polymers may comprise PLA (poy-lactic acid), PGA (poly-glycolic acid), poly lactide-co-glycolides (PLGA), polyalkylcyanoacrylates, poly-e-caprolactones, poly-N-isopropyl acrylamide (NIPA), cellulose ether, hyaluronic acid, lecithin, polyacrylic acid, poly-ε-caprolactone, polyvinylpyrrolidone, polyvinyl alcohol, polyethylene glycol (PEG), and agarose as well as (co)polymer agents obtained by combination or modification of these. The use of polymeric gel delivery system for sustained delivery of drugs provides many advantages and is well known in the art.

Polymers such as PLGA polymers, are progressively eroded when administered into the body. These types of sustained-release formulations have been used to deliver, for example, GnRH analogs over weeks or months. Such formulations have benefits including increased dosage accuracy and improved patient compliance. However there are disadvantages to the use of polymeric hydrogels. Many formulations contain a support material and a pharmaceutical composition, where the support material may have immunogenic properties. Supporting materials may not degrade after administration and will therefore accumulate in the body. Formulations may require complex fabrication or manufacturing procedures. Furthermore, the therapeutic cargo typically represents only a small portion of the therapeutic preparation.

Among the most widely studied self-assembling peptide hydrogels are those made of modified oligopeptides with repeated amino acid sequences, such as those made of tetraphenylethylene-capped dipeptides (e.g., tetraphenylethylene-diglycine (TPE-GG), Yeh et al. 2016 A novel nanostructured supramolecular hydrogel self-assembled from tetraphenylethylene-capped dipeptides. Soft Matter. 12:6347-51; fluorenylmethoxycarbonyl (Fmoc) conjugated-diphenylalanine (Fmoc-FF), Fmoc-tyrosine, or naphthalene-dipeptide hydrogels, Truong et al. 2015 Dissolution and degradation of Fmoc-diphenylalanine self-assembled gels results in necrosis at high concentrations in vitro. Biomater Sci. 3:298-307; Frith et al. 2016 Self-assembly of small peptide amphiphiles, the structures formed and their applications. Philos Trans A Math Phys Eng Sci. 374(2072); Morris et al., 2015 Structural determinants in a library of low molecular weight gelators. Soft Matter. 11:1174-81; Zhou et al. 2014 Extracellular matrix formation in self-assembled minimalistic bioactive hydrogels based on aromatic peptide amphiphiles. J Tissue Eng. 5:2041731414531593; Eckes et al. 2014 β sheets not required: combined experimental and computational studies of self-assembly and gelation of the ester-containing analogue of an Fmoc-dipeptide hydrogelator. Langmuir. 30:5287-96; palmitoyl-V3A3E3 and palmitoyl-A3V3E3, Fu and Nguyen 2015 Sequence-Dependent Structural Stability of Self-Assembled Cylindrical Nanofibers by Peptide Amphiphiles. Biomacromolecules. 16:2209-19).

In addition, a variety of permutations of hydrogel made of complex molecules have been reported. For example, self-assembled gel can be derived from mix of bifunctional peptide LHRH-MPGANLS and siRNA (Liu et al. 2017 Target-specific delivery of siRNA into hepatoma cells' cytoplasm by bifunctional carrier peptide. Drug Deliv Transl Res. 7:147-155), mix of self-assembling peptide and heparin (Liu et al. 2016 Sustained release of hepatocyte growth factor by cationic self-assembling peptide/heparin hybrid hydrogel improves β-cell survival and function through modulating inflammatory response. Int J Nanomedicine. 11:4875-4890), mix of glycosyl-nucleoside and lipid (Kaplan et al. 2016 Self-assembled nanofiber hydrogels for mechanoresponsive therapeutic anti-TNFα antibody delivery. 52:5860-3), use of oxo-ester-mediated chemical ligation to incorporate cysteine, a cysteine-based dipeptide, and a sterically hindered unnatural amino acid (penicillamine) into the peptides (Rasale et al. 2016 Controlling Peptide Self-Assembly through a Native Chemical Ligation/Desulfurization Strategy. Chem Asian J. 11:926-35), and the use of a fusion protein composed of the immunostimulatory Mycobacterium tuberculosis heat shock protein 70 (MtbHSP70) and the biotin binding protein, avidin (Leblanc 2014 VaxCelerate II: rapid development of a self-assembling vaccine for Lassa fever. Hum Vaccin Immunother. 10:3022-38). However, the use of these artificial sequences for drug delivery comes with the risk of eliciting immunogenic responses and causing cytotoxic effect, thereby limiting the usage of these nanostructures in medical applications. In addition, many of these molecules require complex manufacturing processes.

Studies of self-assembled hydrogels showed that the three-dimensional networks of nanofibers of putative gel nanostructures are diverse. For example, the palmitoyl-V3A3E3 gel contains continuous with predominately alkyl tails, whereas the palmitoyl-A3V3E3 gel is disjointed with interconnecting micelles. Likewise, a study of a group of repeat-sequence-containing hydrogel named MAX1 analogs (e.g., VKVKVKVKV(D)PPTKVKVKVKV-NH2) indicated that the self-assembly and hydrogelation rates and mechanical stiffness of the analogs vary drastically despite that all analogs formed networks of entangled fibrils with the similar diameters (Chen et al. 2014 Tuning gelation kinetics and mechanical rigidity of β-hairpin peptide hydrogels via hydrophobic amino acid substitutions. 6:14360-8). These data indicated that the internal structural arrangement of peptide gels and the correlation with structural stability and mechanical behavior of hydrogel nanostructures remains to be fully defined.

The use of gels formed by peptides would provide significant advantages in formulation, however there is a lack of understanding of the requirement for rationale design of self-assembled gel-forming therapeutic peptides. It remains extremely difficult to predict whether a peptide will self-assemble into a gel in aqueous solution, and to design a peptide that forms polymers or a gel by itself at a critical aggregation concentration. Further, peptides can behave differently in solution with different pH, ionic strength, temperature, or co-ingredients. Improved sustained release reagents and formulations as well as therapeutic peptides that can form polymeric gels by itself are urgently needed to improve the delivery of a variety of therapeutics in clinics.

### SUMMARY OF THE INVENTION

Compositions and methods are provided that relate to gel-forming polypeptide therapeutics. Formulations of these gel-forming polypeptides provide advantages, in that therapeutically effective levels of the polypeptide are maintained in vivo for extended periods of time (i.e., increased resident time), thereby increasing treatment intervals and reducing the peak-to-trough effect and total dose of active ingredient, relative to conventional formulations. In some embodiments, methods are provided for formulation and manufacture of such gel-forming peptides. In certain embodiments peptides that naturally form gels are identified, and may be formulated for optimal delivery of the gel-forming peptide and for therapeutic use according to the methods described herein. In other embodiments, peptides that do not naturally form gels, or that only weakly form gels, are modified by joining to a gel-forming-enhancing motif to gain or increase the gel-forming capability, and may be formulated for therapeutic use according to the methods described herein.

In some embodiments, composition and therapeutic formulations are provided, comprising an effective dose of a therapeutic agent in a formulation that adopts a gel configuration on administration. The therapeutic formulation may, for example, provide the therapeutic peptide in a solution or liquid gel, which transitions to a gel upon administration. The resulting gel nanostructure can function as a physical barrier to reduce the diffusion or dispersion of the therapeutic agent, to reduce proteolytic degradation, and to reduce renal clearance of the therapeutic agent, thereby increasing the resident time. In some embodiments the therapeutic agent is a gel-forming polypeptide, which is optionally engineered to enhance gel formation, and reduce the minimal concentration of the gel-forming polypeptide required to provide a gel configuration in vivo. In some embodiments the therapeutic agent is provided in combination with a gel-forming polypeptide. In some embodiments the formulation is free of artificial polymeric gel carrier such as silicon, chitosan, PLA, PGA, PLGA, etc. In some embodiments the formulation is provided in a very low ionicity aqueous solution. In other embodiments the formulation is provided in a two part container, where a dry form of the peptide is mixed immediately prior to administration with an aqueous excipient, which may have very low ionicity.

In some embodiments, gel-forming polypeptides comprise salts of polypeptide hormones and their agonistic, antagonistic, or nonfunctional analogs of ligands for cell surface receptors, e.g. such as GPCRs, tyrosine kinase receptors, etc., and therapeutic agents that act on cell surface targets or enzymes, and salts of biologically active or inactive analogs thereof. Soluble, gel-forming polypeptides also include gel-forming carrier peptides and prodrugs in the form of a gel nanostructure. Gel-forming polypeptides, which comprise a portion of a native polypeptide hormone sequence or analog of a native hormone sequence, can be used as carrier polymers and, once gelled, are capable of controlling the delivery of a therapeutic agent or agents within the gel at a rate suitable for therapeutic use, while avoiding the concern of immunogenic responses.

In some embodiments, a gel-forming polypeptide or combination of polypeptides forms a self-assembled gel in aqueous solution. The gel-forming polypeptide may have a physiologically relevant bioactivity or a therapeutic activity, or may provide a purely structural role. The formulation may comprise additional bioactive molecules. In such formulations, the gel-forming polypeptide in the gel can be released into the physiological environment over an extended period of time, along with the optional additional bioactive agent(s). The gel nanostructure also serves as a physical barrier that decreases the solubility of monomeric peptide and enzymatic degradation of the gel-forming polypeptide.

In some embodiments, a gel-forming therapeutic agent, including without limitation a peptide agent, comprises or is modified to comprise one or more gel-enhancing motifs, which facilitate intermolecular bond formation. An enhancing motif may be a self-assembly-enhancing motif sequence, which promotes self-assembly of polypeptides in solution. The gel-forming motif induces self-assembly of the polypeptide into an aqueous gel. The gel-forming-enhancing motif is optionally derived from a secreted circulating human hormone. The gel-forming-enhancing motif may be fused or otherwise conjugated to a therapeutic agent that functions as a regulator of a cell surface receptor, a biological function, or an enzyme, or as an antigen. The conjugation may be covalent or non-covalent. In some embodiments, a gel-forming polypeptide is modified to comprise a signaling motif that activates or antagonizes a target receptor-mediated signaling pathway, a biological process, or an enzyme reaction. In some embodiments, the therapeutic agent is a small molecule, a peptidomimetic, a biological drug, a nucleic acid, an antigen, an organelle, or a cell.

In some embodiments, a gel-enhancing motif comprises a fragment of a secreted circulating peptide ligand of a cell surface receptor, e.g. a GPCR, including without limitation those peptides identified herein as naturally gel-forming. In some embodiments a gel-enhancing motif is at least about 2, and not more than about 52 amino acids in length, and may be up to 5, up to 7, up to 9, up to 12, up to 15, up to 18, up to 21, up to 24, up to 52 amino acids in length. In some embodiments, the gel-enhancing motif comprises or consists of a fragment of adrenomedullin, adrenomedullin 2, CGRP, or a chimeric polypeptide derived therefrom. In some embodiments, the gel-enhancing motif comprises or consists of a 2-52 amino acid fragment with at least 50%, at least 75%, at least 90% or 100% sequence identity to Pal-KVQKLSAPVDPSSPHSY. In some embodiments, the gel-enhancing motif comprises or consists of a 6-amino-acid segment with at least 50%, at least 75%, at least 90% or 100% sequence identity with Pal-SSPHSY. In some embodiments, the gel-enhancing motif comprises or consists of the 3-amino-acid segment Pal-HSY or Pal-KSY, with the proviso that a peptide naturally containing a Y or SY residue at the amino terminus may be modified by the addition of just those amino acids required to make the HSY sequence. In some embodiments, the gel-enhancing motif comprises or consists of the 2-amino-acid segment Pal-HS, with the proviso that a peptide naturally containing a H, Y, or S residue at the amino terminus may be modified by the addition of just those amino acids required to make the Pal-HS sequence. Alternatively, an amino acid sequence may be substituted at one or more residues to generate the HSY or HS motif. A KSY motif may alternatively be used. In certain embodiments the gel-enhancing motif comprises a conjugated palmitate residue. In certain embodiments the motif is linked to a peptide with mini-PEG at the carboxy or amino terminus, or to a side chain of an amino acid. In some embodiments the peptide comprises a detectable marker, e.g. FITC.

In some embodiments, a therapeutic agent and gel-enhancing motif are covalently linked as portions of a self-assembling gel-forming molecule. In some embodiments, a therapeutic agent is encapsulated by the self-assembling gel-forming polypeptide selected from the list consisting of SEQ ID NOS: 1-15, 48-58, 61, 64, 106-114, 116-124, 126-131, 139-140, and 201-275. In some embodiments, a gel-forming therapeutic; or combination of gel-forming peptide and a therapeutic is administered to a subject and the gel-forming polypeptide/therapeutic localizes in the delivery site and has an increased resident time.

In some embodiments the gel-forming polypeptide is a prodrug that become an active therapeutic agent only after it is dissociated from the gel nanostructure, and carriers of an encapsulated therapeutic agent.

Gel-forming polypeptides include, without limitation, the naturally gel-forming and engineered gel-forming polypeptides set forth in Tables 1, 2 and 3 herein. Such polypeptides include CGRP, ADM, ADM2, Pramlintide, oxytocin, kisspeptin, Pralmorelin, thrombopoietin peptide analog, Romiplostim analog, urocortin 3, Substance P, GLP-1, GnRH analog, and GLP-2 receptor ligand analog, a bombesin receptor antagonist, gamma-MSH, and gel-forming ligands for opioid receptors, and their analogs thereof; etc. Also included are polypeptides and analogs thereof that activate compstatin- and thymosin alpha 1-mediated pathways, thymosin beta 4-mediated pathways, and their analogs thereof. Gel-forming polypeptides also include polypeptides that contain a self-assembly-enhancing motif conjugated to a functional sequence, which include without limitation ADM, ADM2, GnRH, GnRH antagonist, vasopressin, oxytocin, apelin, neurotensin, kisspeptin, bombesin, deltorphin, enkephalin, substance P, saralasin, calcitonin, Pramlintide (amylin analog), exenatide 4, GLP-1, Teduglutide (GLP-2 analog), afamelanotide (melanotan I), melanotan II, gamma-MSH, ACTH1-24, setmelanotide, PYY3-36, urocortin 2, urocortin 3, parathyroid hormone, HOE140 (a BKR2 antagonist), bradykinin receptor 1 antagonist (BKR1 antagonist), Pralmorelin, sermorelin, atrial natriuretic peptide (ANP), thymosin alpha-1, thymosin beta 4, vasoactive intestinal peptide (VIP), TAT cell-penetrating-enhancing peptide, a kallikrein inhibitor, antimicrobial peptide such as temporin A, Glatiramer peptide (or Copaxone), matrix modifying peptide 1, matrix modifying peptide 4, matrix modifying peptide 7, matrix modifying peptide 8, acetyl hexapeptide-3 matrix modifying peptide, insulin, relaxins, PTHrP, bobmesin receptor antagonist, and their analogs thereof.

In one embodiment, a method is provided for treating a patient with a gel-forming formulation, the method comprising administering an effective dose and concentration of a therapeutic agent in a formulation that adopts a gel configuration on administration. The therapeutic formulation may be a liquid solution or a liquid gel prior to administration. The therapeutic formulation may be administered by, for example, intramuscular, subcutaneous, intradermal, or intraperitoneal injection, infusion, or administered intranasally, intrauterinely, intraocularly, topically, orally, or intrarectally, wherein the composition forms a gel after interaction with the patient's bodily fluids.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-1C. Graphic representation of a peptide gel formed by SEQ ID NO: 1 peptide.

FIG. 2. Bull frogs treated with the MSH gel made of SEQ ID NO: 224 had lasting color change when compared to those treated with the wild-type analog SEQ ID NO: 29 (100 nmoles/kg body weight).

### DETAILED DESCRIPTION

Compositions and methods are provided for the formulation and use of gel-forming polypeptides. When administering a therapeutic agent as a gel, cost-effective processes can be used for formulation, preparation and manufacture. The immunogenicity of the formulation is low, as the gel-forming polypeptides are derived from simple secreted human peptide hormone sequences. The formulations allow the therapeutic agents to retain a volume of distribution similar to that of their wild-type or native counterparts. The composition according to the present invention may be prepared by simply mixing the active ingredient and an aqueous solution, thus dramatically reducing the volume, cost, and manufacturing time for these therapeutic candidates when compared to the preparation made with known sustained-release formulations.

Without being limited by the theory, it has been discovered that certain secreted polypeptide hormones can self-assemble to form gel nanostructures without the addition of an artificial polymer or other carrier matrix to control the peptide's release profile. These peptides may contain a gel-forming-enhancing motif, and automatically gel upon interaction with an aqueous solution. Peptide formulations that exploit this ability dramatically increase the loading capacity of a therapeutic agent, and at the same time reduce the cost and manufacturing time as compared to known sustained-release artificial polymer-based formulations. Moreover, the use of these gel-forming polypeptides eliminates the need to use organic solvents in the formulation of a sustained release formulation.

GPCRs and other surface receptors play a pervasive physiological role and are the leading target class for pharmaceuticals. Researchers have designed novel agonists and inhibitors of a variety of GPCRs and other cell surface receptors to allow the regulation of receptor signaling in patients, however a major drawback of the usage of many polypeptide and nonpolypeptide therapeutics is their short half-life and resident time in vivo. The short half-life is commonly associated with a sensitivity to proteolysis and/or renal clearance. An important technique to lengthen in vivo half-life and resident time is the use of self-assembling peptide gel formulations. Unlike the use of artificial polymer molecules such as PEG, PLA, PGA, PLGA, or collagen to encapsulate a therapeutic, certain therapeutic polypeptides such as lanreotide can self-assemble into gel nanostructures in aqueous solution. After injecting into a patient, the lanreotide gel then gradually release the lanreotide monomer from the gel depot, allowing the sustained release of lanreotide in the circulation. This allows the prolonged modulation of somatostatin receptors in patients.

Peptides that are identified to form gels by themselves can be formulated to extend the resident time of these molecules in vivo. Peptides that do not naturally form gels can be engineered by conjugation to gel-enhancing sequence motifs. The gel-enhancing motif can be conjugated to any part of a therapeutic to promote gel-forming capability without dramatically changing the mass of the resulting molecules. Gel-enhancing motifs can be used to promote gel-forming capability of small molecule drugs, peptides, proteins, or other biomolecules and extend the resident time in vivo.

Another embodiment of the present invention includes compositions comprising first self-assembling peptide incorporating a first biological signal and a second self-assembling peptide incorporating a second biological signal.

### Definitions

The practice of the present invention may employ conventional techniques of molecular biology (including recombinant techniques), microbiology, cell biology, biochemistry, peptide chemistry and immunology within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction” (Mullis et al., eds., 1994); and “Current Protocols in Immunology” (J. E. Coligan et al., eds., 1991); as well as updated or revised editions of all of the foregoing.

Although specific peptides are exemplified herein, any of a number of alternative peptides and methods apparent to those of skill in the art upon contemplation of this disclosure are equally applicable and suitable for use in practicing the invention. The methods of the invention, as well as tests to determine their efficacy in a particular patient or application, can be carried out in accordance with the teachings herein using procedures standard in the art. Thus, the practice of the present invention may employ conventional techniques of molecular biology (including recombinant techniques), microbiology, cell biology, biochemistry and immunology within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction” (Mullis et al., eds., 1994); and “Current Protocols in Immunology” (J. E. Coligan et al., eds., 1991); as well as updated or revised editions of all of the foregoing.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, primates and humans.

The term “gel” refers to a liquid, semi-solid, or solid three-dimensional network that spans the volume of a liquid medium. A hydrogel is a network of polymer chains that are hydrophilic, sometimes found as a colloidal gel in which water is the dispersion medium. Hydrogels are highly absorbent, and may comprise greater than 50% water, greater than 60%, greater than 70%, greater than 80%, and may be from about 85% to about 99.9% water. Hydrogels are characterized by an elastic modulus which exhibits a pronounced plateau extending to times at least of the order of seconds, and by a viscous modulus which is considerably smaller than the elastic modulus in the plateau region. The elastic modulus of hydrogels is generally within 10-10² kPa range. Because they easily break and slump under their own weight, tensile and bending tests are not suitable configurations to assess elastic modulus. Methods known in the art may be used to characterize gels described herein, e.g. see Kocen et al. (2017) Biomedical Materials 12(2), “Viscoelastic behaviour of hydrogel-based composites for tissue engineering under mechanical load” and Jonker et al. (2012) Chem. Mater. 24 (5), pp 759-773, “Peptide- and Protein-Based Hydrogels”.

What is meant by a “clear aqueous solution” is a solution comprised of a flowing aqueous solution wherein preferably 95%±5% of the polypeptide solute is completely dissolved so that the solution is relatively transparent and freely movable with a tilt of the container or a snap of the finger. A clear solution may have trace amounts of observable solute or particles depending on the purity of the solutes used. However, such particles are not sufficient to create a milky or cloudy appearance. On the other hand, the gel-forming polypeptides assume a liquid gel or semisolid gel appearance. The most critical criterium for this assessment is the increase in aqueous viscosity. A liquid gel appearance is that exhibit consistant increase in viscosity and to exhibit a liquid gel as exemplified by a honey or glycerine. The liquid gel could be clear or exhibit an opaque appearance. A liquid gel does not apply to a suspension which is a heterogeneous mixture composed of insoluble particles. A liquid gel could as a continuous phase, whereas the gel is a homogeneous, single-phase solution with high viscosity, but does reach a semisolid appearance.

A semisolid gel is a formulation comprised of a solvent and one or more solutes wherein the solute may be completely or partially dissolved, so that the formulation assumes a transparent or consistent opaque appearance, and the high viscosity render the solution rather immovable upon the snap of a finger or a tilt of the container. The semisolid gel composition does not apply to a suspension which is a heterogeneous mixture composed of high levels of observable insoluble particles, whereas a solution is not homogeneous.

An insoluble precipitates by a suspension or a solution is an aqueous formulation comprised of one or more solutes wherein the solute is not dissolved or partially dissolved, so that the formulation is not a consistent composition that could be as liquid as a clear or opaque solution or more viscous as a gel.

The terms “semisolid suspension” and “semisolid therapeutic composition” are used interchangeably herein to refer to viscous, paste-like suspension of therapeutic polypeptides in a liquid solvent, such as water. A semisolid suspension according to the invention includes (1) a semisolid, soluble, gel-forming polypeptide particles and up to 50 percent, by weight, of a pharmaceutically acceptable formulation to provide the semisolid consistency. The salt form of the gel-forming polypeptides that can be used in the compositions of the invention should gel in bodily fluids when administered to a patient, and, once gelled, are capable of sustained delivery of the peptide at a rate suitable for a therapeutic use of the drug.

A self-assembled gel-forming polypeptide is a polypeptide that forms a gel upon contact with an aqueous solution, including for example physiological fluids such as blood, etc. Self-assembly peptide gel formation has been defined as the spontaneous organization of peptides in solution via non-covalent interactions (Whitesides et al.; Science 1991, 254, 1312-1319). These gels create a nanostructure that modify rheological properties.

It has been proposed that peptide gels belong to two major categories: one mainly consists of sequences of alternating hydrophobic and hydrophilic amino acids (an amphiphilic peptide), and the other is peptide with a hydrophobic group and forms nanofibers (a peptide amphiphile)(Mata et al., 2010 Biomaterials 31, 6004; Shah et al., 2010 P Natl Acad Sci USA 107, 3293; Huang et al. 2010 Biomaterials 31, 9202; Webber et al., 2011 P Natl Acad Sci USA 108, 13438. Capito, et. al.; 2008 Science 319, 1812-1816). An example of peptide amphiphiles with alkyl tails on one terminus include amphiphiles derived from peptide motifs found in collagen. A peptide amphiphile is usually formed through hydrogen-bonding between beta-sheet forming amino acids and hydrophobic interactions of the tails. Alternatively, peptides consisting of alternating hydrophobic and hydrophilic amino acids can self-assemble into gels with parallel or anti-parallel beta sheets. Thus, peptide gels may include extended cross-beta structure that form tubes and fibers, and alpha-helices that form triple-helix collagen-like structures, helix barrels, or coiled-coil bundles.

Self-assembly of gels is typically driven by a combination of hydrogen bonds and Van der Waals interactions between monomeric molecules (Reches and Gazit; Curr. Nanoscience 2006, 2, 105-111). Once in solution, the gel-formation potential of the self-assembling peptide monomer increases rapidly until a critical concentration is reached. Solvent conditions including pH, salt concentration, dielectric property and temperature can alter the self-assemble process of almost all gel-forming peptides (Zhang et al., 2010 Nature Materials, 9, 594-601). For polypeptides containing charged residues, high salt concentration can lower the critical aggregation concentration (Ellis-Behnke et. al. 2006 Nanomedicine: Nanotechnology, Biology and Medicine 2, 207-215). Likewise, the pH can affect the self-assemble property of peptides (Aggeli et al.; Angew. Chem. Int. Ed. 2003, 42, 5603-5606).

Growth factors have been physically entrapped in polypeptide hydrogels, either covalently linked or bound electrostatically to anionic polymers or structures such as heparin. Drawbacks to these and related systems include non-specificity of bound growth factors or a requirement for degradation of covalent bonds to achieve the desired effect. Further, these peptides may incur adverse immunogenic responses after administration due to artificial sequences with repeated motifs (e.g., the FKFEFKFE motif- and RADARADA motif-containing gel-forming peptides), or derivation from large proteins such as fibronectin and laminin or from noncirculating proteins such as amyloid protein. A peptide gel made of native or modified secreted circulating polypeptide sequences, as described herein, has a lower potential for immunogenic response and a better safety profile. Because most polymeric carriers usually represent 75-99% of the mass in sustained release formulation, the use of a gel-forming therapeutics can increase the effective load of therapeutic drugs by more than 20-50 fold.

The term “sustained release” as used herein means release of the active substance or a carrier material in a patient such that the patient receives a dose of the therapeutic substance over a prolonged period of time. The proportion of therapeutic agents in the composition that will be released in a time window will be determined by the release rate which it is desired to achieve.

Aqueous excipients. In some embodiments of the invention, gel-forming peptides are formulated for delivery in an aqueous excipient, where the excipient may be very low ionicity. Such excipients may be defined as less than about 50 mM total ion concentration, less than about 25 mM total ion concentration, less than about 15 mM total ion concentration, less than about 10 mM total ion concentration, less than about 5 mM total ion concentration, less than about 2.5 mM total ion concentration, less than about 1 mM total ion concentration, less than about 0.1 mM total ion concentration, less than about 0.01 mM total ion concentration, less than about 0.001 mM total ion concentration, less than about 0.0001 mM total ion concentration, less than about 0.00001 mM total ion concentration. The salts that are present may include, for example, Na⁺, K⁺, Cl⁻, Mn⁺⁺, Mg⁺⁺, Ca⁺⁺, PO₄⁻, etc.

The term “carrier peptide” refers to a gel-forming peptide that will form a gel in aqueous solution or upon contact with bodily fluids, and that can be used to encapsulate or associate with therapeutic agents for sustained delivery.

The term “prodrug peptide” refers to a gel-forming peptide that cannot act on a biological target while in the gel nanostructure and only becomes active upon dissociation from the gel nanostructure.

A gel-enhancing motif is an amino acid sequence that enhances the ability of a small molecule, a peptide, a biological, an antigen, a nucleotide, or a therapeutic molecule to form a gel nanostructure in aqueous solutions. A gel-enhancing motif may comprise or consist of a fragment of a secreted circulating peptide ligand of a cell surface receptor. In some embodiments a gel-enhancing motif is at least about 2, and not more than about 52 amino acids in length, and may be up to 5, up to 7, up to 9, up to 12, up to 15, up to 18, up to 21, up to 24, up to 52 amino acids in length. In some embodiments, the gel-enhancing motif comprises or consists of a fragment of adrenomedullin, adrenomedullin 2, CGRP, or a chimeric polypeptide derived therefrom. In some embodiments, the gel-enhancing motif comprises or consists of a 2-52 amino acid fragment with at least 50%, at least 75%, at least 90% or 100% sequence identity to Pal-KVQKLSAPVDPSSPHSY. In some embodiments, the gel-enhancing motif comprises or consists of a 6-amino-acid segment with at least 50%, at least 75%, at least 90% or 100% sequence identity with Pal-SSPHSY. In some embodiments, the gel-enhancing motif comprises or consists of the 3-amino-acid segment Pal-HSY or Pal-KSY, with the proviso that a peptide naturally containing a Y or SY residue at the amino terminus may be modified by the addition of just those amino acids required to make the HSY sequence. In some embodiments, the gel-enhancing motif comprises or consists of the 2-amino-acid segment Pal-HS, with the proviso that a peptide naturally containing a H, Y, or S residue at the amino terminus may be modified by the addition of just those amino acids required to make the Pal-HS sequence. Alternatively, an amino acid sequence may be substituted at one or more residues to generate the HSY or HS motif. A KSY motif may alternatively be used. In certain embodiments the gel-enhancing motif comprises a conjugated palmitate residue. In certain embodiments the motif is linked to a peptide with mini-PEG at the carboxy or amino terminus, or to a side chain of an amino acid. In some embodiments the peptide comprises a detectable marker, e.g. FITC.

In addition to the gel-enhancing amino acid sequence, additional agents can be conjugated to a therapeutic polypeptide to enhance gel formation, including without limitation fatty acids, pegylation, and the like. The addition of fatty acids can enhance the gel-forming capability, and enhance subsequent dissociation of the nanostructure due to the presence of chemical bonds that are susceptible to esterase-mediated cleavage of the fatty acid.

The term PEGylated as used herein refers to polypeptides that are chemically modified with one or more polyethylene glycol moieties, i.e., PEGylated. The polypeptide may be coupled directly to PEG (i.e., without a linking group) through an amino group, a sulfhydryl group, a hydroxyl group, or a carboxyl group. In some embodiments, the PEGylated polypeptide contains a PEG moiety on only one amino acid. In other embodiments, the PEGylated polypeptide contains a PEG moiety on two or more amino acids.

In some embodiments, PEG is attached to the polypeptide via a linking group. The linking group is any biocompatible linking group, where “biocompatible” indicates that the compound or group is non-toxic and may be utilized in vitro or in vivo without causing injury, sickness, disease, or death. PEG can be bonded to the linking group, for example, via an ether bond, an ester bond, a thiol bond or an amide bond. Suitable biocompatible linking groups include, but are not limited to, an ester group, an amide group, an imide group, a carbamate group, a carboxyl group, a hydroxyl group, a carbohydrate, a succinimide group (including, for example, succinimidyl succinate (SS), succinimidyl propionate (SPA), succinimidyl butanoate (SBA), succinimidyl carboxymethylate (SCM), succinimidyl succinamide (SSA) or N-hydroxy succinimide (NHS)), an epoxide group, an oxycarbonylimidazole group (including, for example, carbonyldimidazole (CDI)), a nitro phenyl group (including, for example, nitrophenyl carbonate (NPC) or trichlorophenyl carbonate (TPC)), a trysylate group, an aldehyde group, an isocyanate group, a vinylsulfone group, a tyrosine group, a cysteine group, a histidine group or a primary amine. If an intact, properly folded polypeptide protein is reacted with the PEG coupling reagent, then the PEG groups will preferentially react with surface residues as opposed to buried residues, which provides practical, cost-efficient procedures for protein PEGylation and synthesis of the PEGylated polypeptides of the invention.

Methods for attaching a PEG to a polypeptide are known in the art, and any known method can be used in accordance with the methods of the invention to produce a PEGylated polypeptide of the invention. See, for example, by Park et al, Anticancer Res., 1:373-376 (1981); Zaplipsky and Lee, Polyethylene Glycol Chemistry: Biotechnical and Biomedical Applications, J. M. Harris, ed., Plenum Press, NY, Chapter 21 (1992); U.S. Pat. Nos. 5,985,265; 5,672,662 (Harris, et al.) and WO 97/03106.

In many embodiments, the PEG is a monomethoxy PEG molecule that reacts with primary amine groups on the polypeptide. Methods of modifying polypeptides with monomethoxy PEG via reductive alkylation are known in the art. See, e.g., Chamow et al. (1994) Bioconj. Chem. 5:133-140.

Polyethylene glycol suitable for conjugation to a polypeptide is soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons.

In many embodiments, PEG has at least one hydroxyl group, e.g., a terminal hydroxyl group, which hydroxyl group is modified to generate a functional group that is reactive with an amino group, e.g., an epsilon amino group of a lysine residue, a free amino group at the N-terminus of a polypeptide, or any other amino group such as an amino group of asparagine, glutamine, arginine, or histidine, to facilitate covalent modification of a polypeptide with PEG.

In other embodiments, PEG is derivatized so that it is reactive with free carboxyl groups in the polypeptide. Suitable derivatives of PEG that are reactive with the free carboxyl group at the carboxyl-terminus of polypeptide include, but are not limited to PEG-amine, and hydrazine derivatives of PEG (e.g., PEG-NH—NH₂).

In other embodiments, PEG is derivatized such that it comprises a terminal thiocarboxylic acid group, —COSH, which selectively reacts with amino groups to generate amide derivatives. Because of the reactive nature of the thio acid, selectivity of certain amino groups over others is achieved. For example, —SH exhibits sufficient leaving group ability in reaction with N-terminal amino group at appropriate pH conditions such that the ε-amino groups in lysine residues are protonated and remain non-nucleophilic. On the other hand, reactions under suitable pH conditions may make some of the accessible lysine residues react with selectivity.

In other embodiments, the PEG comprises a reactive ester such as an N-hydroxy succinimidate at the end of the PEG chain. Such an N-hydroxysuccinimidate-containing PEG molecule reacts with select amino groups at particular pH conditions such as neutral 6.5-7.5. For example, the N-terminal amino groups may be selectively modified under neutral pH conditions. However, if the reactivity of the reagent were extreme, accessible-NH₂ groups of lysine may also react.

In some embodiments, the PEG conjugated to the polypeptide polypeptide is linear. In other embodiments, the PEG conjugated to the polypeptide polypeptide is branched. Branched PEG derivatives such as those described in U.S. Pat. No. 5,643,575, “star-PEG's” and multi-armed PEG's such as those described in Shearwater Polymers, Inc. catalog “Polyethylene Glycol Derivatives 1997-1998.” Star PEGs are described in the art including, e.g., in U.S. Pat. No. 6,046,305.

PEG having a molecular weight in a range of from about 0.2 kDa to about 100 kDa, is generally used, where the term “about,” in the context of PEG, indicates that in preparations of polyethylene glycol, some molecules will weigh more, some less, than the stated molecular weight. For example, PEG suitable for conjugation to polypeptide has a molecular weight of from about 0.2 kDa to about 5 kDa, from about 5 kDa to about 10 kDa, from about 10 kDa to about 15 kDa, from about 15 kDa to about 20 kDa, from about 20 kDa to about 25 kDa, from about 25 kDa to about 30 kDa, from about 30 kDa to about 40 kDa, from about 40 kDa to about 50 kDa, from about 50 kDa to about 60 kDa, from about 60 kDa to about 70 kDa, from about 70 kDa to about 80 kDa, from about 80 kDa to about 90 kDa, or from about 90 kDa to about 100 kDa.

Peptides may be conjugated to fatty acids, including without limitation conjugation at the amino terminus, for example where a linear or branched C₃-C₁₀₀ alkyl; preferably a C₄-C₃₀ alkyl optionally substituted with halo, hydroxy, alkoxy, amino, alkylamino, dialkylamino, sulfate, or phosphate, and which may by saturated, or mono- or di-unsaturated, e.g. 18:0, 24:0 and 24:1. Fatty acids of interest include, without limitation, palmitic acid; stearic acid; arachidic acid; lauric acid; myristic acid; myristoleic acid; palmitoleic acid; sapienic acid; oleic acid; linoleic acid; α-linolenic acid; arachidonic acid; eicosapentaenoic acid; erucic acid; docosahexaenoic acid; etc.

In one embodiment, the gel-forming polypeptide comprises a homolog, a variant, or a functional fragment of the wild-type counterpart peptide, including ligands for GPCR. In another embodiment, the gel-forming polypeptide comprises an amino acid sequence that is about 70%, 75%, 80%, 85%, 90%, or 95% identical to a wild-type, i.e. naturally-occurring counterpart peptide. The gel-forming polypeptide may have the sequence set forth in, for example, SEQ ID NOS:1-15, 48-58, 61, 64, 106-114, 116-124, 126-131, 139-140, 201-275, and derivatives thereof.

The term gel-forming polypeptide ligand, as used herein, may refer to any polypeptide analogs which exhibit gel-forming capability at a concentration equal or lower than the concentration that allows the wild-type counterparts to form a gel in the sam aqueous solution, or wild-type and modified polypeptides that are identified to form gel at 6, 11, 20, or 30% w/w. In one embodiment, gel-forming polypeptides of the present invention, however, can be shorter or longer than the SEQ ID NOS: 1-15, 48-58, 61, 64, 101-140 and 201-275, e.g. truncated by 1, 2, 3, 4, 5, 6, 7, 8, 9, 10 or more residues at the amino and/or carboxy terminus, with substitution of nonnative amino acids or fused to an additional sequence.

### Compositions

A peptide gel is a smart delivery system comprising biodegradable and biocompatible polypeptide(s), and is accepted by regulatory authorities for application in drug delivery. Peptide gels exhibit superior advantages over other colloidal carriers such as nanoemulsions, polymeric nanoparticles, liposomes, and solid lipid nanoparticles etc. One of the foremost qualifications is the high drug delivery capacity (i.e., enhanced drug loading capacity and increased drug bioavailability) and the lack of immunogenicity. In addition, this formulation reduces issues associated with production and suitable formulations. However, the mechanisms of self-assemble of polypeptides into gel nanostructure is not well understood, and minor changes in amino acid composition and other modifications may alter a polypeptide's tendency to self-assemble into a gel nanostructure.

Described herein are gel-forming polypeptides; and therapeutic compositions comprising at least one polypeptide with or without a gel-enhancing motif, wherein the enhancing motif is a component of a secreted polypeptide hormone that enhances gel formation. The composition may further include a functional therapeutic component, or the therapeutic component may be provided by the gel-forming peptide. Included as formulations are stable aqueous solutions, gels and liquid gels, said formulations comprising at least about 0.01%, at least about 0.1%, at least about 1%, at least about 5%, at least about 10%, at least 12%, at least 15%, at least 18%, at least 20%, at least 30% or more (w/w) of a gel-forming polypeptide compound. The formulation may further comprise a very low ionicity aqueous excipient. Methods are also provided for preparing a stable aqueous formulation of a gel-forming polypeptide, comprising dissolving at least about 0.01%, at least about 0.1%, at least about 1%, at least about 5%, at least about 10%, at least 12%, at least 15%, at least 18%, at least 20%, at least 30% or more or more (w/w) of a gel-forming polypeptide in a low ionicity aqueous excipient. The formulation may be administered to an individual suffering from a condition that may be alleviated by administration of a therapeutic polypeptide gel, said methods comprising administering to said subject an effective amount of the stable aqueous formulation.

The compositions and methods described herein provide novel and improved self-assembling gel-forming polypeptides, comprising a gel-enhancing motif, improved gel-forming polypeptide carriers, improved usage of a therapeutic agent via the formation of a gel nanostructure, and other self-assembling nanostructures and methods of making and using same. The gel-forming therapeutic formulations utilize noncovalent electrostatics to control the solubility and nanostructure of therapeutic molecules or carrier polypeptides which may or may not contain a gel-forming-enhancing motif.

Physicochemical properties (e.g., swelling behavior), mechanical properties (e.g, compressive modulus), degradation rates as well as active agent release kinetics of the subject hydrogels can be modulated by varying the amount of polypeptide present. For example, the percentage can be varied between about 0.01% and about 50% by weight, and, such as from about 0.02% and about 45% by weight, such as from about 0.03% and 40% by weight, such as from 0.04% to 35% by weight and including from about 0.05% to 30% by weight.

As noted above, the physicochemical and mechanical properties as well as the active agent release kinetics of the subject hydrogels may vary depending on hydrogel structure. The subject compositions absorb solvent (e.g. water) and undergo swelling under nonphysiological condition (e.g., in pure water) or physiological conditions (e.g., in contact with blood or plasma). The term “swelling” as referred to herein is meant the isotropic (or anisotropic) expansion of the hydrogel structure as solvent (e.g., water) molecules diffuse throughout the internal volume of the hydrogel. Depending on the structure of the hydrogels, the swelling ratio may vary. By “swelling ratio” is meant the ratio of the hydrogel weight after absorption of solvent to the dry weight of the hydrogel.

Likewise, the compressive modulus of the hydrogels may vary depending on the composition of the hydrogel. By compressive modulus is meant the capacity of the subject hydrogels to withstand axially directed pushing forces and is the value of uniaxial compressive stress reach when the material fails completely (e.g., crushed). In some embodiments, the compressive modulus of the subject hydrogels could range from 0.1 kPa to 35 kPa, such as from 0.2 kPa to 33 kPa, such as from 0.3 kPa to 30 kPa, such as from 0.4 kPa to 28 kPa, such as form 0.5 kPa to 25 kPa, such as from 0.6 kPa to 22 kPa, such as from 0.7 kPa to 20 kPa and including a compressive modulus ranging from 1.0 kPa to 20 kPa.

The pore sizes of the hydrogel may also vary depending on the structure of the hydrogel. In some embodiments, the pore sizes of the hydrogel ranges from 0.01 microns to 1000 microns, such as 0.05 microns to 900 microns, such as 0.1 micron to 800 microns, such as 0.5 microns to 750 microns, such as 1.0 microns to 600 microns, such as 2.5 microns to 500 microns, such as 5.0 microns to 400 microns and including from 10.0 microns to 300 microns.

The rate of degradation of hydrogels under physiological conditions may vary depending on the structure and composition. In some embodiments, the subject hydrogels are structurally designed to degrade under physiological conditions (e.g., in vivo) over a predetermined duration, such as for example 0.5 days or longer, such as 1 day or longer, such as 2 days or longer, such as 5 days or longer, such as 7 days or longer, such as 10 days or longer, such as 14 days or longer, such as 21 days or longer, such as 28 days or longer, such as 70 days or longer and including 100 days or longer. In other embodiments, hydrogels of interest are configured to degrade when exposed to physiological conditions at a predetermined rate, such as at a substantially zero-order degradation rate, such as at a substantially first order degradation rate and including at a substantially second-order degradation rate.

In one embodiment, the self-assembling gel-forming polypeptides comprise an or more acyl group to improve the gel-forming capability and to add susceptibility to esterase enzyme reaction.

In some embodiments, the gel-enhancing motif is a sequence derived from an agonist or ligand of a cell surface receptor selected from LHRH receptor, vasopressin receptors, oxytocin receptors, apelin receptor, neurotensin receptors, kisspeptin receptor, bombesin receptors, delta opioid receptor, mu opoid receptor, kappa opioid receptor, substance P receptors, angiotensin II receptors, calcitonin receptor, amylin receptors, GLP-1 receptor, GLP-2 receptor, glucagon receptor, calcitonin gene related peptide (CGRP) receptors, adrenomedullin receptors, melanocortin receptors, parathyroid hormone receptors, bradykinin receptors, neuropeptide Y (NPY) receptor, peptide YY (PYY) receptor, vasoactive intestinal polypeptide (VIP) receptor, urocortin receptors, somatostatin receptors, endothelin receptors, adrenocorticotropic hormone (ACTH) receptors, melanocyte stimulating hormone (MSH) receptors, growth hormone-releasing hormone receptors, ghrelin receptors, glucagon receptor, PTHrP receptor, insulin receptors, relaxin receptors, natriuretic peptide receptors, erythropoietin receptors.

In some embodiments, the gel-enhancing motif is a sequence derived from an agonist or ligand of human CLR/RAMP receptors. In some embodiments, the gel-enhancing motif is a sequence derived from human adrenomedullin, adrenomedullin 2, and CGRP. In some embodiments, the gel-enhancing motif is a sequence derived from human adrenomedullin and/or adrenomedullin 2.

In some embodiments the resulting structure has the formula

Ea-(Fa)n-Ga (II)

wherein Ea is a gel-forming-enhancing cell-surface-receptor-ligand-derived polypeptide motif or a therapeutic agent; Fa is a PEG group or a linker sequence; n is an integral number from 0 to 40; and Ga is a therapeutic agent, or a gel-forming-enhancing cell-surface-receptor-ligand-derived polypeptide motif.

In some embodiments, a gel-forming-enhancing motif is conjugated to a therapeutic agent selected from the list consisting of small molecules, polypeptides, proteins, enzymes, hormones, polynucleotides, nucleoproteins, polysaccharides, glycoproteins, lipoproteins, steroids, analgesics, local anesthetics, antibiotics, chemotherapeutic, immunosuppressive agents, anti-inflammatory, antiproliferative, antimitotic, angiogenic, antiangiogenic, antipsychotic, central nervous system (CNS), anticoagulant, and fibrinolytic drugs; said drugs include LHRH analogs, LHRH antagonist analogs, vasopressin analogs, oxytocin analogs, apelin analogs, neurotensin analogs, kisspeptin analogs, kisspeptin 234 analogs, bombesin analogs, bombesin receptor antagonists, bradykinin analogs, bradykinin receptor antagonist analogs, opioid analogs, deltorphin analogs, encephalin analogs, substance P analogs, angiotensin II analogs, parathyroid hormone analogs, PTHrP analogs, GLP-1 analogs, GLP-2 analogs, glucagon analogs, GIP analogs, calcitonin analogs, amylin analogs, CGRP analogs, adrenomedullin analogs, adrenomedullin 2 analogs, neuropeptide Y (NPY) analogs, peptide YY (PYY) analogs, NPY antagonist analogs, vasoactive intestinal polypeptide (VIP) analogs, urocortin analogs, urocortin 2 analogs, urocortin 3 analogs, bradykinin analogs, somatostatin analogs, endothelin analogs, adrenocorticotropic hormone (ACTH) analogs, melanotan I analogs, melanotan II analogs, melanocyte stimulating hormone (MSH) analogs, melanocortin analogs, growth hormone-releasing hormone analogs, ghrelin analogs, HOE140 analogs, insulin analogs, relaxin analogs, atrial natriuretic peptide (ANP) analogs, brain natriuretic peptide (BNP) analogs, C-type natriuretic peptide (CNP) analogs, glatiramer peptide (copaxone), thymosin alpha-1 analogs, thymosin beta 4 analogs, cell-penetrating peptides, TAT peptide, kallikrein inhibitors, compstatins, antimicrobial temporin YIGSR peptide, RGD peptide, VGVAPG peptide, EEMQRR peptide, and YRSRKYSSWY peptide; and blood clotting factors, cytotoxic therapeutics, microbial antigens, viral antigens, tumor antigens, neoantigens, and cosmetheutical peptides and pharmaceutically acceptable salts of these compounds.

In some embodiments, the target of the therapeutic agent of the present invention is a cell surface receptor (e.g., opioid receptors and Romiplostim receptor) or enzyme (e.g., in the cases of kallikrein inhibitor or compstatin). In some embodiments, the therapeutic target is a biological function mediator (e.g., in the case of Glatiramer peptide, which has a less well-defined target and is thought to act by modifying immune processes that are currently believed to be responsible for the pathogenesis of multiple sclerosis; in the case of thymosin alpha 1 and thymosin beta 4, they are believed to enhance cell-mediated immunity in humans as well as experimental animal). In some embodiments, the therapeutic target is the cell membrane barrier (e.g., in the cases of antimicrobial temporin A derivative, a cell-penetrating peptide such as the TAT cell-penetrating sequence). In some embodiments, the therapeutic target is the skin matrix components and matrix enzymes (e.g., matrix modifying proteins 1, 4, 7, 8 and acetyl hexapeptide-3 matrix modifying peptide).

The following are non-limiting examples of the polypeptides useful for gel-forming, and for use in therapeutic gel-forming formulations.

A CLR/RAMP receptor ligand of a gel-forming polypeptide comprises a homolog, a variant, a chimera, or a functional fragment of adrenomedullin, adrenomedullin 2, and CGRP, for example SEQ ID NOS:1-3, 48-58, and 106-114, 116-124, 126-131, 139-140, and 274-275 including without limitation the sequence of SEQ ID NOS:1-3, 48-58, 106-114, 116-124, 126-131, 139-140, and 274-275. In another embodiment, the gel-forming polypeptide comprises an amino acid sequence that is about 70%, 75%, 80%, 85%, 90%, or 95% identical to SEQ ID NOS: 1-3, 48-58, 106-114, 116-124, 126-131, 139-140, and 274-275. The term CLR/RAMP receptor ligand, as used herein, may refer to any functional peptide analog which activates or inhibits a CLR/RAMP receptor (CLR/RAMP1, 2 and 3). In one embodiment, the CLR/RAMP receptor ligand is an analog of adrenomedullin, adrenomedullin 2, and CGRP, or SEQ ID NOS: 1-3, 48-58, 106-114, 116-124, 126-131, 139-140, and 274-275. The CLR/RAMP receptor ligand within the gel-forming polypeptide of the present invention, however, can be shorter or longer (e.g., 5-50 or more amino acids in length).

In some embodiments, an ADM, CGRP, or IMD sequence comprises a structure of Formula I: R1-B0-B1-B2-B3-B4-B5-B6-B7-B8-B9-B10-B11-B12-B13-B14-B15-B16-B17-B18-B19-B20-B21-B22-B23-B24-B25-B26-B27-B28-R2, where:

R1 a functional group comprising a structure of Formula (W′)(X′)n(Y′)n(Z′)n, wherein W is a fatty acid, a fatty diacid, a fatty acid or cholesterol derivative or empty; X′ is a PEG group, glutamic acid, γ-glutamic acid, a non-proteinogenic amino acid, or empty; Y′ is a PEG group, glutamic acid, γ-glutamic acid, a non-proteinogenic amino acid, or empty; Z′ is a proteinogenic amino acid, a non-proteinogenic amino acid, or empty;

R2 is an C-terminal modification including an {NH2} amidation, {—CHO} peptide aldehydes, {-ol} alcohol peptide, {CMK} chloromethylketone, {FMK} Fluoromethylketone, {Cya} Cysteamide, {pNA} p-nitroaniline, {—ONP} para-nitrophenol, {AMC} 7-Amino-4-methylcoumarin, {AFC}, —OMe (C-terminal), —OEt (C-terminal), —OBzl (C-terminal), —OtBu (C-terminal), {—OSu} hydroxysuccinimide ester, —NHMe (C-terminal), NHEt (C-terminal), —NHisopen (C-terminal), NH(CH2)6 (C-terminal), —NHPh (C-terminal), {NHEt(O)EtNH-Fmoc} 2,2′-Oxydi Ethanamine-Fmoc, {NHEt(EtNH-Myr)2}, —NH(OMe)Me (C-terminal), -TBzl (C-terminal), —NHNH2 (C-terminal), -ED (C-terminal) —NH—CH2CH2-NH2, or -BD (C-terminal) —NH—CH2CH2CH2CH2-NH2NH2 group;

B0 is selected from the group consisting of an empty residue, any proteinogenic amino acid or non-proteinogenic amino acid, acylated histidine (acy-His), acylated arginine (acy-Arg), acylated lysine (acy-Lys);

B1 is selected from the group consisting of an empty residue, Val, Ala, Gly, Ile, Leu, His, Arg, Lys, Asn, Gln and a non-proteinogenic amino acid;

B2 is selected from the group consisting of an empty residue, Arg, Lys, Gln, Glu, Asp, Asn and a non-proteinogenic amino acid;

B3 is selected from the group consisting of an empty residue, Ala, Leu, Ile, Val, Met, Phe, His, Arg, Lys, Gln, Asp and a non-proteinogenic amino acid;

B4 is selected from the group consisting of an empty residue, Val, Ala, Gly, Ile, Leu and a non-proteinogenic amino acid;

B5 is selected from the group consisting of an empty residue, Val, Ala, Gly, Ile, Leu, Pro, Ser, Th, Tyr and a non-proteinogenic amino acid;

B6 is selected from the group consisting of an empty residue, Ala, Leu, Ile, Val, Met, Phe, His, Arg, Lys and a non-proteinogenic amino acid;

B7 is selected from the group consisting of an empty residue, Ala, Leu, Ile, Val, Met, Phe, Gln, Asn, His, Arg, Lys and a non-proteinogenic amino acid;

B8 is selected from the group consisting of an empty residue, Val, Ala, Gly, Ile, Leu, Ser, Thr and a non-proteinogenic amino acid;

B9 is selected from the group consisting of an empty residue, Arg, Lys, Asn, Gln, Trp, Phe, Ser, Thr, Tyr and a non-proteinogenic amino acid;

B10 is selected from the group consisting of an empty residue, Ala, Ser, Thr, Gln, Glu, Asp, Asn and a non-proteinogenic amino acid;

B11 is selected from the group consisting of an empty residue, Trp, Phe, Val, Ala, Gly, Ile, Leu, Pro and a non-proteinogenic amino acid;

B12 is selected from the group consisting of an empty residue, Ala, Gly, Ser, Thr, Pro, Tyr; Met, Trp, Phe and a non-proteinogenic amino acid;

B13 is selected from the group consisting of an empty residue, Gln, Glu, Asp, and Asn, Val, Ala, Gly, Ile, Leu, Met, Phe and a non-proteinogenic amino acid;

B14 is selected from the group consisting of an empty residue, His, Arg, Lys, Val, Ala, Gly, Ile, Leu, Met, Phe, Pro and a non-proteinogenic amino acid;

B15 is selected from the group consisting of an empty residue, Arg, Lys, Gln, Glu, Asp, Asn, Val, Ala, Gly, Ile, Leu and a non-proteinogenic amino acid;

B16 is selected from the group consisting of an empty residue, Asn, Gln, Val, Ala, Gly, Ile, Leu and a non-proteinogenic amino acid;

B17 is selected from the group consisting of an empty residue, Asn, Gln, Ser, Thr, Tyr and a non-proteinogenic amino acid;

B18 is selected from the group consisting of an empty residue, Val, Ala, Gly, Ile, Leu, Phe, Tyr and a non-proteinogenic amino acid;

B19 is selected from the group consisting of an empty residue, Val, Ala, Gly, Ile, Leu, Met, Phe, Pro and a non-proteinogenic amino acid;

B20 is selected from the group consisting of an empty residue, His, Arg, Lys, Val, Ala, Gly, Ile, Leu, Pro and a non-proteinogenic amino acid;

B21 is selected from the group consisting of an empty residue, Ile, Val, Ser, Thr, Tyr, Gln, Glu, Asp, Asn and a non-proteinogenic amino acid;

B22 is selected from the group consisting of an empty residue, His, Arg, Lys, Val, Ala, Gly, Ile, Leu, Asn, Gln, Pro and a non-proteinogenic amino acid;

B23 is selected from the group consisting of an empty residue, Ser, Thr, Tyr, Val, Ala, Gly, Ile, Leu, Met, Phe and a non-proteinogenic amino acid;

B24 is selected from the group consisting of an empty residue, Ala, Gly, Pro, Ser, Thr, Tyr and a non-proteinogenic amino acid;

B25 is selected from the group consisting of an empty residue, Val, Ala, Gly, Ile, Leu, Pro, Ser, Thr and a non-proteinogenic amino acid;

B26 is selected from the group consisting of an empty residue, His, Arg, Lys, Gln, Glu, Asp, Asn and a non-proteinogenic amino acid;

B27 is selected from the group consisting of an empty residue, Val, Ala, Gly, Ile, Leu, Ser, Thr, Tyr and a non-proteinogenic amino acid;

B28 is selected from the group consisting of an empty residue, Ala, Leu, Ile, Val, Phe, Ser, Thr, Tyr and a non-proteinogenic amino acid.

A GnRH receptor ligand component of a gel-forming polypeptide comprises a homolog, a variant, an analog, a chimera, or a functional fragment of a GnRH receptor agonist or antagonist, for example SEQ ID NO:15, or may comprise or consist of SEQ ID NO:15. In another embodiment, the gel-forming GnRH receptor ligand comprises an amino acid sequence that is about 70%, 75%, 80%, 85%, 90%, or 95% identical to SEQ ID NO:15 or SEQ ID NO:201, or may comprise or consist of SEQ ID NO:15 or SEQ ID NO:201. The term GnRH receptor ligand, as used herein, may refer to any functional analog (e.g., Buserelin, Deslorelin, Fertirelin, Goserelin, Leuprorelin, Nafarelin, and Triptorelin) which is capable of activating or inhibiting a GnRH receptor. A GnRH receptor ligand within the gel-forming polypeptide of the present invention, however, can be shorter (e.g., 6-9 or less amino acids in length) or longer (e.g., 11-40 or more amino acids in length).

A GnRH receptor antagonist component of a gel-forming polypeptide comprises a homolog, a variant, an analog, a chimera, or a functional fragment of a GnRH receptor antagonist, for example SEQ ID NO:27, including without limitation the sequence of SEQ ID NO:27. In another embodiment, a gel-forming GnRH receptor antagonist comprises an amino acid sequence that is about 70%, 75%, 80%, 85%, 90%, or 95% identical to SEQ ID NO: 202 or 203, or may or may comprise or consist of SEQ ID NO:202 or 203. The term GnRH receptor antagonist, as used herein, may refer to any functional analog (e.g., Abarelix, Cetrorelix, Degarelix, Ganirelix, and Ozarelix) which inhibits a GnRH receptor. In one embodiment, the GnRH receptor antagonist is an analog of a GnRH receptor antagonist or SEQ ID NO:27. The GnRH receptor antagonist within the gel-forming polypeptide of the present invention, however, can be shorter or longer (e.g., 5-40 or more amino acids in length) than the provided sequence.

A vasopressin receptor ligand component of a gel-forming polypeptide comprises a homolog, a variant, an analog, a chimera, or a functional fragment of a vasopressin receptor agonist or antagonist, for example SEQ ID NO:17, including without limitation the sequence of SEQ ID NO:17. In another embodiment, the a gel-forming vasopressin receptor ligand comprises an amino acid sequence that is about 70%, 75%, 80%, 85%, 90%, or 95% identical to SEQ ID NO: 204, or may comprise or consist of SEQ ID NO:204. The term vasopressin receptor ligand, as used herein, may refer to any functional analog (e.g., desmopressin, lypressin, argipressin, d[Leu4,Lys8]-VP, (d(CH2)51,Tyr(Me)2,Arg8)-vasopressin, and pitressin) which activates or inhibits a vasopressin receptor. In one embodiment, the vasopressin receptor ligand is an analog of a vasopressin receptor ligand or SEQ ID NO:17. The vasopressin receptor ligand within the gel-forming polypeptide of the present invention, however, can be shorter or longer (e.g., 5-40 or more amino acids in length) than the provided sequences.

An oxytocin receptor ligand component of a gel-forming polypeptide comprises a homolog, a variant, an analog, a chimera, or a functional fragment of a oxytocin receptor agonist or antagonist, for example SEQ ID NO:5, including without limitation the sequence of SEQ ID NO:5. In another embodiment, the a gel-forming oxytocin receptor ligand comprises an amino acid sequence that is about 70%, 75%, 80%, 85%, 90%, or 95% identical to SEQ ID NO: 5 or 205, or may comprise or consist of SEQ ID NO:5 or SEQ ID NO:205. The term oxytocin receptor ligand, as used herein, may refer to any functional analog (e.g., demoxytocin, merotocin, oxytocin, WAY-267,464, pitocin, barusiban, atosiban, and carbetocin) which activates or inhibits an oxytocin receptor. In one embodiment, the oxytocin receptor ligand is an analog of an oxytocin receptor agonist, antagonist, or SEQ ID NO:5. The oxytocin receptor ligand within the gel-forming polypeptide of the present invention, however, can be shorter or longer (e.g., 5-40 or more amino acids in length) than the provided sequence.

An apelin receptor (i.e., APJ receptor and Apela/ELABELA/Toddler) ligand component of a gel-forming polypeptide comprises a homolog, a variant, an analog, a chimera, or a functional fragment of an apelin receptor agonist or antagonist, for example SEQ ID NO:18, including without limitation the sequence of SEQ ID NO:18. In another embodiment, the gel-forming apelin receptor ligand comprises an amino acid sequence that is about 70%, 75%, 80%, 85%, 90%, or 95% identical to SEQ ID NOS: 206 and 262, or may comprise or consist of SEQ ID NOS: 206 and 262. The term apelin receptor ligand, as used herein, may refer to any functional analog (e.g., apelin 36), apelin 17, apelin 13, and Apela/ELABELA/Toddler) which activates or inhibits an apelin receptor. In one embodiment, the apelin receptor ligand is an analog of an apelin receptor agonist, antagonist, or SEQ ID NO:18. The apelin receptor ligand within the gel-forming polypeptide of the present invention, however, can be shorter or longer (e.g., 5-40 or more amino acids in length) than the provided sequence.

A neurotensin receptor (NTSR1 and NTSR2) ligand component of a gel-forming polypeptide comprises a homolog, a variant, an analog, a chimera, or a functional fragment of a neurotensin receptor agonist or antagonist, for example SEQ ID NO:19, including without limitation the sequence of SEQ ID NO:19. In another embodiment, the gel-forming neurotensin receptor ligand comprises an amino acid sequence that is about 70%, 75%, 80%, 85%, 90%, or 95% identical to SEQ ID NO:207, or may comprise or consist of SEQ ID NO:207. The term neurotensin receptor ligand, as used herein, may refer to any functional ananlog (e.g, Beta-lactotensin, JMV-449, Neurotensin 13, Neuromedin N, Xenin 8, Kinetensin, PD-149,163, Levocabastine, SR-48692, and SR-142,948) which is capable of activating or inhibiting a neurotensin receptor. In one embodiment, the neurotensin receptor ligand is a neurotensin 13 analog or SEQ ID NO:19. A neurotensin receptor ligand within the gel-forming polypeptide of the present invention, however, can be shorter (e.g., 6-12 or less amino acids in length) or longer (e.g., 14-40 or more amino acids in length) than the provided sequence.

A kisspeptin receptor (GPR54) ligand component of a gel-forming polypeptide comprises a homolog, a variant, an analog, a chimera, or a functional fragment of a kisspeptin receptor agonist or antagonist, for example SEQ ID NOS:6, including without limitation the sequence of SEQ ID NOS:6. In another embodiment, the a gel-forming kisspeptin receptor ligand comprises an amino acid sequence that is about 70%, 75%, 80%, 85%, 90%, or 95% identical to SEQ ID NOS: 6 or 208, or may comprise or consist of SEQ ID NO: 6 or 208. The term kisspeptin receptor ligand, as used herein, may refer to any functional analog (e.g., kisspeptin-10, kisspeptin-13, kisspeptin 17, and kisspeptin 234 antagonist) which activates or inhibits a kisspeptin receptor. In one embodiment, the kisspeptin receptor ligand is an analog of a kisspeptin receptor agonist, antagonist, or SEQ ID NO:6. The kisspeptin receptor ligand within the gel-forming polypeptide of the present invention, however, can be shorter or longer (e.g., 5-40 or more amino acids in length) than the provided sequence.

A bombesin receptor (BB1, BB2 and BB3 receptors) ligand component of a gel-forming polypeptide comprises a homolog, a variant, an analog, a chimera, or a functional fragment of a bombesin receptor agonist or antagonist, for example SEQ ID NOS:20 and 61, including without limitation the sequence of SEQ ID NOS:20 and 61. In another embodiment, the a gel-forming bombesin receptor ligand comprises an amino acid sequence that is about 70%, 75%, 80%, 85%, 90%, or 95% identical to SEQ ID NOS: 61, 209 or 255, or may comprise or consist of SEQ ID NO:61, 209 or 255. The term bombesin receptor ligand, as used herein, may refer to any functional analog (e.g., bombesin, neuromedin B, BIM 187, BIM 189, [D-Phe12,Leu14]-bombesin, alytesin, BIM 23042, [D-Phe12,Leu14]-bombesin, and gastrin-releasing peptide) which activates or inhibits a bombesin receptor. In one embodiment, the bombesin receptor ligand is an analog of a bombesin receptor agonist, antagonist, or SEQ ID NO:20 and 61. The bombesin receptor ligand within the gel-forming polypeptide of the present invention, however, can be shorter or longer (e.g., 5-40 or more amino acids in length) than the provided sequence.

A deltorphin or an opioid receptor (i.e., delta opioid receptor, kappa opioid receptor, mu opioid receptor and nociceptin receptor) ligand component of a gel-forming polypeptide comprises a homolog, a variant, an analog, a chimera, or a functional fragment of an opioid receptor agonist or antagonist, for example SEQ ID NO:21, including without limitation the sequence of SEQ ID NO:21. In another embodiment, the a gel-forming opioid receptor ligand comprises an amino acid sequence that is about 70%, 75%, 80%, 85%, 90%, or 95% identical to SEQ ID NO: 211, 212, 213, or 214, or may comprise or consist of SEQ ID NO:211, 212, 213, or 214. The term opioid receptor ligand, as used herein, may refer to any functional analog (e.g., buprenorphin, Leu-enkephalin, Met-enkephalin, deltorphins, DADLE, DPDPE, 7-spiroindanyloxymorphone, and N-phenethyl-14-ethoxymetopon) which activates or inhibits an opioid receptor. In one embodiment, the opioid receptor ligand is an analog of an opioid receptor agonist, antagonist, or SEQ ID NO:21. The opioid receptor ligand within the gel-forming polypeptide of the present invention, however, can be shorter or longer (e.g., 4-40 or more amino acids in length) than the provided sequence.

An enkephalin or an opioid receptor (i.e., delta opioid receptor, kappa opioid receptor, mu opioid receptor and nociceptin receptor) ligand component of a gel-forming polypeptide comprises a homolog, a variant, an analog, a chimera, or a functional fragment of an opioid receptor agonist or antagonist, for example SEQ ID NO:22, including without limitation the sequence of SEQ ID NO:22. In another embodiment, the a gel-forming opioid receptor ligand comprises an amino acid sequence that is about 70%, 75%, 80%, 85%, 90%, or 95% identical to SEQ ID NO: 211, 212, 213, or 214, or may comprise or consist of SEQ ID NO:211, 212, 213, or 214. The term opioid receptor ligand, as used herein, may refer to any functional analog (e.g., Leu-enkephalin, Met-enkephalin, deltorphins, DADLE, DPDPE, 7-spiroindanyloxymorphone, and N-phenethyl-14-ethoxymetopon) which activates or inhibits an opioid receptor. In one embodiment, the opioid receptor ligand is an analog of an opioid receptor agonist, antagonist, or SEQ ID NO:22. The opioid receptor ligand within the gel-forming polypeptide of the present invention, however, can be shorter or longer (e.g., 4-40 or more amino acids in length) than the provided sequence.

A kappa opioid receptor ligand component of a gel-forming polypeptide comprises a homolog, a variant, an analog, a chimera, or a functional fragment of a kappa opioid receptor agonist or antagonist, for example SEQ ID NO:7, including without limitation the sequence of SEQ ID NO:7. In another embodiment, the a gel-forming kappa opioid receptor ligand comprises an amino acid sequence that is about 70%, 75%, 80%, 85%, 90%, or 95% identical to SEQ ID NO: 7 or 214, or may comprise or consist of SEQ ID NO:7 or SEQ ID NO:214. The term kappa opioid receptor ligand, as used herein, may refer to any functional analog (e.g., CR665, difelikefalin (CR845), and dynorphins) which activates or inhibits a kappa opioid receptor. In one embodiment, the kappa opioid receptor ligand is an analog of a kappa opioid receptor agonist, antagonist, or SEQ ID NO:7 or 214. The kappa opioid receptor ligand within the gel-forming polypeptide of the present invention, however, can be shorter or longer (e.g., 4-40 or more amino acids in length) than the provided sequence.

A substance P receptor (or neurokinin 1 receptor) ligand component of a gel-forming polypeptide comprises a homolog, a variant, an analog, a chimera, or a functional fragment of a substance P receptor agonist or antagonist, for example SEQ ID NO:23, including without limitation the sequence of SEQ ID NO:23. In another embodiment, the a gel-forming substance P receptor ligand comprises an amino acid sequence that is about 70%, 75%, 80%, 85%, 90%, or 95% identical to SEQ ID NO:215, 253 and 254, or may comprise or consist of SEQ ID NO:215, 253, and 254. The term substance P receptor ligand, as used herein, may refer to any functional analog (e.g, substance P, GR-73632, Aprepitant, Casopitant, Ezlopitant, Fosaprepitant, Lanepitant, Maropitant, and Vestipitant) which activates or inhibits a substance P receptor. In one embodiment, the substance P receptor ligand is an analog of a substance P receptor agonist, antagonist, or SEQ ID NO:23. The substance P receptor ligand within the gel-forming polypeptide of the present invention, however, can be shorter or longer (e.g., 5-40 or more amino acids in length) than the provided sequence.

An angiotensin II receptor ligand component of a gel-forming polypeptide comprises a homolog, a variant, an analog, a chimera, or a functional fragment of an angiotensin II receptor agonist or antagonist, for example SEQ ID NO:24, including without limitation the sequence of SEQ ID NO:24. In another embodiment, the a gel-forming angiotensin II receptor ligand comprises an amino acid sequence that is about 70%, 75%, 80%, 85%, 90%, or 95% identical to SEQ ID NO:216, or may comprise or consist of SEQ ID NO:216. The term angiotensin II receptor ligand, as used herein, may refer to any functional analog (e.g., saralasin) which activates or inhibits an angiotensin II receptor. In one embodiment, the angiotensin II receptor ligand is an analog of an angiotensin II receptor agonist, antagonist, or SEQ ID NO:24. The angiotensin II receptor ligand within the gel-forming polypeptide of the present invention, however, can be shorter or longer (e.g., 5-40 or more amino acids in length) than the provided sequence.

A calcitonin receptor ligand component of a gel-forming polypeptide comprises a homolog, a variant, an analog, a chimera, or a functional fragment of a calcitonin receptor agonist or antagonist, for example SEQ ID NO:28, including without limitation the sequence of SEQ ID NO:28. In another embodiment, the a gel-forming calcitonin receptor ligand comprises an amino acid sequence that is about 70%, 75%, 80%, 85%, 90%, or 95% identical to SEQ ID NO:217, or may comprise or consist of SEQ ID NO:217. The term calcitonin receptor ligand, as used herein, may refer to any functional analog (e.g., Miacalcin analogs) which activates or inhibits a calcitonin receptor. In one embodiment, the calcitonin receptor ligand is an analog of a calcitonin receptor agonist, antagonist, or SEQ ID NO:28. The calcitonin receptor ligand within the gel-forming polypeptide of the present invention, however, can be shorter or longer (e.g., 5-40 or more amino acids in length) than the provided sequence.

An amylin receptor (calcitonin receptor/RAMP1, 2 and 3) ligand component of a gel-forming polypeptide comprises a homolog, a variant, an analog, a chimera, or a functional fragment of an amylin receptor agonist or antagonist, for example SEQ ID NO:4, including without limitation the sequence of SEQ ID NO:4. In another embodiment, the a gel-forming amylin receptor ligand comprises an amino acid sequence that is about 70%, 75%, 80%, 85%, 90%, or 95% identical to SEQ ID NO:4, or 218, or may comprise or consist of SEQ ID NO:4, or 218. The term amylin receptor ligand, as used herein, may refer to any functional analog which (e.g., Pramlintide) activates or inhibits an amylin receptor. In one embodiment, the amylin receptor ligand is an analog of an amylin receptor agonist, antagonist, or SEQ ID NO:4. The amylin receptor ligand within the gel-forming polypeptide of the present invention, however, can be shorter or longer (e.g., 5-40 or more amino acids in length) than the provided sequence.

A GLP-1 receptor ligand component of a gel-forming polypeptide comprises a homolog, a variant, an analog, a chimera, or a functional fragment of a GLP-1 receptor agonist or antagonist, for example SEQ ID NOS:14 and 26, including without limitation the sequence of SEQ ID NOS:14 and 26. In another embodiment, the a gel-forming GLP-1 receptor ligand comprises an amino acid sequence that is about 70%, 75%, 80%, 85%, 90%, or 95% identical to SEQ ID NOS:14, 219, 220, -221, or 269-272, or may comprise or consist of SEQ ID NO:14, 219, 220, 221, or 269-272. The term GLP-1 receptor ligand, as used herein, may refer to any functional analog (e.g., GLP-1 7-37, exenatide, [Glu13] exenatide, [Met(O)14]-exenatide, [N-acetyl-His1]-exenatide, liraglutide, lixisenatide, albiglutide, dulaglutide, semaglutide, and taspoglutide) which activates or inhibits a GLP-1 receptor. In one embodiment, the GLP-1 receptor ligand is an analog of a GLP-1 receptor agonist, antagonist, or SEQ ID NO:14 or 26. The GLP-1 receptor ligand within the gel-forming polypeptide of the present invention, however, can be shorter or longer (e.g., 5-40 or more amino acids in length) than the provided sequence.

A GLP-2 receptor ligand component of a gel-forming polypeptide comprises a homolog, a variant, an analog, a chimera, or a functional fragment of a GLP-2 receptor agonist or antagonist, for example SEQ ID NO:12, including without limitation the sequence of SEQ ID NO:12. In another embodiment, the a gel-forming GLP-2 receptor ligand comprises an amino acid sequence that is about 70%, 75%, 80%, 85%, 90%, or 95% identical to SEQ ID NO:12 or 222, or may comprise or consist of SEQ ID NO:12 or 222. The term GLP-2 receptor ligand, as used herein, may refer to any functional analog (e.g., Tedglutide, and GLP2) which activates or inhibits a GLP-2 receptor. In one embodiment, the GLP-2 receptor ligand is an analog of a GLP-2 receptor agonist, antagonist, or SEQ ID NO:12. The GLP-2 receptor ligand within the gel-forming polypeptide of the present invention, however, can be shorter or longer (e.g., 5-40 or more amino acids in length) than the provided sequence.

A melanocortin receptor (i.e., MC1R-MC5R) ligand component of a gel-forming polypeptide comprises a homolog, a variant, an analog, a chimera, or a functional fragment of a melanocortin receptor agonist or antagonist, for example SEQ ID NOS:29, 33, 34, 62, and 64 including without limitation the sequence of SEQ ID NOS: 29, 33, 34, 62, and 64. In another embodiment, the gel-forming melanocortin receptor ligand comprises an amino acid sequence that is about 70%, 75%, 80%, 85%, 90%, or 95% identical to SEQ ID NO: 64, 223, 224, 225, 226, 227, 228, 256, 257, 259, 260, or 273, or may comprise or consist of SEQ ID NO:64, 223, 224, 225, 226, 227, 228, 256, 257, 259, 260, or 273. The term melanocortin receptor ligand, as used herein, may refer to any functional analog which activates or inhibits a melanocortin receptor (e.g., α-MSH, β-MSH, γ-MSH, ACTH1-24, cosyntropin, afamelanotide, BMS-470,539, bremelanotide, Melanotan II, modimelanotide, setmelanotide, PF-00446687, PL-6983, THIQ, PF-219,061, UK-414,495, agouti-related peptide, and agouti signaling peptide). In one embodiment, the melanocortin receptor ligand is an analog of a melanocortin receptor agonist, antagonist, or SEQ ID NO: 29, 33, 34, 62, or 64. The melanocortin receptor ligand within the gel-forming polypeptide of the present invention, however, can be shorter or longer (e.g., 4-40 or more amino acids in length) than the provided sequence.

A neuropeptide Y receptor (NPY1R, NPY2R, PPYR1, NPY5R) ligand component of a gel-forming polypeptide comprises a homolog, a variant, an analog, a chimera, or a functional fragment of a neuropeptide Y receptor agonist or antagonist, for example SEQ ID NOS:30 including without limitation the sequence of SEQ ID NOS: 30. In another embodiment, the gel-forming neuropeptide Y receptor ligand comprises an amino acid sequence that is about 70%, 75%, 80%, 85%, 90%, or 95% identical to SEQ ID NO:229 or 264, or may comprise or consist of SEQ ID NO:229, or 264. The term neuropeptide Y receptor ligand, as used herein, may refer to any functional analog (e.g, Neuropeptide Y fragment 13-36, Peptide YY, Peptide YY 3-36 fragment, [Leu31,Pro34]-Neuropeptide Y, Neuropeptide Y, BVD-10, GR-231,118, [cPP1-7,NPY19-23,Ala31,Aib32,Gln34]-hPancreatic polypeptide, BVD 10, and Pancreatic polypeptide) which activates or inhibits a neuropeptide Y receptor. In one embodiment, the neuropeptide Y receptor ligand is an analog of a neuropeptide Y receptor agonist, antagonist, or SEQ ID NO:30. The neuropeptide Y receptor ligand within the gel-forming polypeptide of the present invention, however, can be shorter or longer (e.g., 5-40 or more amino acids in length) than the provided sequence.

A corticotropin receptor (CRHR1 and CRHR2) ligand component of a gel-forming polypeptide comprises a homolog, a variant, an analog, a chimera, or a functional fragment of a corticotropin receptor agonist or antagonist, for example SEQ ID NOS:10 and 31, including without limitation the sequence of SEQ ID NOS: 10 and 31. In another embodiment, the gel-forming corticotropin receptor ligand comprises an amino acid sequence that is about 70%, 75%, 80%, 85%, 90%, or 95% identical to SEQ ID NO:10, 230, 231, or 265, or may comprise or consist of SEQ ID NO: 10, 230, 231, or 265. The term corticotropin receptor ligand, as used herein, may refer to any functional analog (CRH, urocortin 1, urocortin 2, urocortin3, Stressin 1, Antalarmin hydrochloride, Antisauvagine-30, LWH-234, CP-154,526, NBI-27914, R-121,919, Astressin-2B, and Astressin-B) which activates or inhibits a corticotropin receptor. In one embodiment, the corticotropin receptor ligand is an analog of a corticotropin receptor agonist, antagonist, CRH, urocortin 1, urocortin 2, urocortin3, or SEQ ID NO: 10 or 31. The corticotropin receptor ligand within the gel-forming polypeptide of the present invention, however, can be shorter or longer (e.g., 5-50 or more amino acids in length).

A parathyroid hormone receptor (PTH1R and PTH2R) ligand component of a gel-forming polypeptide comprises a homolog, a variant, an analog, a chimera, or a functional fragment of a parathyroid hormone receptor agonist or antagonist, for example SEQ ID NOS:25 including without limitation the sequence of SEQ ID NO:25. In another embodiment, the gel-forming parathyroid hormone receptor ligand comprises an amino acid sequence that is about 70%, 75%, 80%, 85%, 90%, or 95% identical to SEQ ID NO:232, 258, or may comprise or consist of SEQ ID NO:232, 258. The term parathyroid hormone receptor ligand, as used herein, may refer to any functional analog (e.g., parathyroid hormone, parathyroid hormone-related protein, DPC AJ1951, Teriparatide, and Abaloparatide) which activates or inhibits a parathyroid hormone receptor. In one embodiment, the parathyroid hormone receptor ligand is an analog of a parathyroid hormone receptor agonist, antagonist, or SEQ ID NO:25. The parathyroid hormone receptor ligand within the gel-forming polypeptide of the present invention, however, can be shorter or longer (e.g., 5-50 or more amino acids in length) than the provided sequences.

A bradykinin receptor (BDKRB1 and BDKRB2) ligand component of a gel-forming polypeptide comprises a homolog, a variant, an analog, a chimera, or a functional fragment of a bradykinin receptor agonist or antagonist, for example SEQ ID NOS:32 and 65; Firazyr or icatibant, including without limitation the sequence of SEQ ID NOS:32 and 65. In another embodiment, the gel-forming bradykinin receptor ligand comprises an amino acid sequence that is about 70%, 75%, 80%, 85%, 90%, or 95% identical to SEQ ID NO: 233, 234, or 261, or may comprise or consist of SEQ ID NO:233, 234, or 261. The term bradykinin receptor ligand, as used herein, may refer to any functional analog (e.g., bradykinin1-8, [Leu8]-bradykinin1-8, Sar-[D-Phe8]-des-Arg9-bradykinin, KRPPGFS-DβNal-I, [Phe8ψ(CH—NH)-Arg9]-bradykinin, MEN 11270, R 715, R 892, and Hoe 140 (icatibant)) which activates or inhibits a bradykinin receptor. In one embodiment, the bradykinin receptor ligand is an analog of a bradykinin receptor antagonist or SEQ ID NO:32. The bradykinin receptor ligand within the gel-forming polypeptide of the present invention, however, can be shorter or longer (e.g., 5-40 or more amino acids in length) than the provided sequence.

A ghrelin/growth hormone secretagogue receptor (GHSR) ligand component of a gel-forming polypeptide comprises a homolog, a variant, an analog, a chimera, or a functional fragment of a ghrelin/growth hormone secretagogue receptor (GHSR) agonist or antagonist, for example SEQ ID NO:8, including without limitation the sequence of SEQ ID NO:8. In another embodiment, the gel-forming ghrelin/growth hormone secretagogue receptor ligand comprises an amino acid sequence that is about 70%, 75%, 80%, 85%, 90%, or 95% identical to SEQ ID NO:8 or 235, or may comprise or consist of SEQ ID NO:8 or 235. The term ghrelin/growth hormone secretagogue receptor (GHSR) ligand, as used herein, may refer to any functional analog (e.g., Anamorelin, Capromorelin, Examorelin (hexarelin), ghrelin (lenomorelin), GHRP-6, Ibutamoren (MK-677), Ipamorelin, Macimorelin, Pralmorelin (GHRP-2), Relamorelin, SM-130,686, Tabimorelin, and Ulimorelin) which activates or inhibits a ghrelin/growth hormone secretagogue receptor. In one embodiment, the ghrelin/growth hormone secretagogue receptor ligand is an analog of a ghrelin/growth hormone secretagogue receptor agonist, antagonist, or SEQ ID NO:8. The ghrelin/growth hormone secretagogue receptor (GHSR) ligand within the gel-forming polypeptide of the present invention, however, can be shorter or longer (e.g., 5-50 or more amino acids in length) than the provided sequences.

A growth hormone-releasing hormone receptor (GHRHR) ligand component of a gel-forming polypeptide comprises a homolog, a variant, an analog, a chimera, or a functional fragment of a growth hormone-releasing hormone receptor agonist or antagonist, for example SEQ ID NO:35, including without limitation the sequence of SEQ ID NO:35. In another embodiment, the gel-forming ghrelin/growth hormone secretagogue receptor ligand comprises an amino acid sequence that is about 70%, 75%, 80%, 85%, 90%, or 95% identical to SEQ ID NO:35, 236, or 237, or may comprise or consist of SEQ ID NO:35, 236 or 237. The term growth hormone-releasing hormone receptor ligand, as used herein, may refer to any functional analog (e.g., CJC-1295, Dumorelin, GHRH (somatorelin), Rismorelin, Sermorelin (GHRH 1-29), and Tesamorelin) which activates or inhibits a growth hormone-releasing hormone receptor. In one embodiment, the growth hormone-releasing hormone receptor ligand is an analog of a growth hormone-releasing hormone receptor agonist, antagonist, or SEQ ID NO:35. The growth hormone-releasing hormone receptor ligand within the gel-forming polypeptide of the present invention, however, can be shorter or longer (e.g., 5-50 or more amino acids in length) than the provided sequences.

A vasoactive intestinal peptide receptor (VIPR) ligand component of a gel-forming polypeptide comprises a homolog, a variant, an analog, a chimera, or a functional fragment of a vasoactive intestinal peptide receptor agonist or antagonist, for example SEQ ID NO:67, including without limitation the sequence of SEQ ID NO:67. In another embodiment, the gel-forming VIP receptor ligand comprises an amino acid sequence that is about 70%, 75%, 80%, 85%, 90%, or 95% identical to SEQ ID NO:266, or may comprise or consist of SEQ ID NO:266. The term VIP receptor ligand, as used herein, may refer to any functional analog which activates or inhibits a VIP receptor. In one embodiment, the VIP receptor ligand is an analog of a VIP receptor agonist, antagonist, or SEQ ID NO:67. The VIP receptor ligand within the gel-forming polypeptide of the present invention, however, can be shorter or longer (e.g., 5-50 or more amino acids in length) than the provided sequences.

A natriuretic peptide receptor (i.e., NPR1, NPR2, and NPR3) ligand component of a gel-forming polypeptide comprises a homolog, a variant, an analog, a chimera, or a functional fragment of a natriuretic peptide receptor agonist or antagonist, for example SEQ ID NOS:41, including without limitation the sequence of SEQ ID NOS:41. In another embodiment, the gel-forming natriuretic peptide receptor ligand comprises an amino acid sequence that is about 70%, 75%, 80%, 85%, 90%, or 95% identical to SEQ ID NO:238, or may comprise or consist of SEQ ID NO:238. The term natriuretic peptide receptor ligand, as used herein, may refer to any functional analog (e.g., atrial natriuretic peptide, brain natriuretic peptide, C-type natriuretic peptide, and nesiritide) which activates or inhibits a natriuretic peptide receptor. In one embodiment, the natriuretic peptide receptor ligand is an analog of a natriuretic peptide receptor agonist, antagonist, atrial natriuretic peptide, brain natriuretic peptide, C-type natriuretic peptide, or SEQ ID NO:41. The natriuretic peptide receptor ligand within the gel-forming polypeptide of the present invention, however, can be shorter or longer (e.g., 5-50 or more amino acids in length).

A thymosin α1-like ligand component of a gel-forming polypeptide comprises a homolog, a variant, an analog, a chimera, or a functional fragment of a thymosin α1 and thymosin α1-like ligand, for example SEQ ID NO:13, including without limitation the sequence of SEQ ID NO:13. In another embodiment, the gel-forming thymosin α1-like ligand comprises an amino acid sequence that is about 70%, 75%, 80%, 85%, 90%, or 95% identical to SEQ ID NO:13, 239, 240, 267, or 268, or may comprise or consist of SEQ ID NO:13, 239, 240, 267, or 268. The term thymosin α1-like ligand, as used herein, may refer to any functional analog (e.g., thymosin α1) which activates or inhibits the thymosin α1-mediated signaling pathway. In one embodiment, the thymosin α1 and thymosin α1-like ligand is an analog of thymosin α1, or SEQ ID NO:13. The thymosin α1-like ligand within the gel-forming polypeptide of the present invention, however, can be shorter or longer (e.g., 5-50 or more amino acids in length).

A thymosin beta 4 ligand component of a gel-forming polypeptide comprises a homolog, a variant, an analog, a chimera, or a functional fragment of a thymosin beta 4 ligand, for example SEQ ID NO:66, including without limitation the sequence of SEQ ID NO:66. In another embodiment, the gel-forming thymosin beta 4-like ligand comprises an amino acid sequence that is about 70%, 75%, 80%, 85%, 90%, or 95% identical to SEQ ID NO:263, or may comprise or consist of SEQ ID NO:263. The term thymosin beta 4, as used herein, may refer to any functional analog (e.g., thymosin beta 4) which activates or inhibits the thymosin beta 4-mediated signaling pathway. In one embodiment, the thymosin beta 4 and thymosin beta 4-like ligand is an analog of thymosin beta 4, or SEQ ID NO:263. The thymosin beta 4-like ligand within the gel-forming polypeptide of the present invention, however, can be shorter or longer (e.g., 5-50 or more amino acids in length).

A cell-penetrating peptide component of a gel-forming polypeptide comprises a homolog, a variant, an analog, a chimera, or a functional fragment of cell-penetrating peptide, for example SEQ ID NO:36, including without limitation the sequence of SEQ ID NO:36. In another embodiment, the gel-forming cell-penetrating peptide comprises an amino acid sequence that is about 70%, 75%, 80%, 85%, 90%, or 95% identical to SEQ ID NO:241, or may comprise or consist of SEQ ID NO:241. The term cell-penetrating peptide, as used herein, may refer to any functional peptide analog which promotes the transfer of a molecule from the extracellular space to the intracellular space (please see examples in Kalafatovic D and Giralt E 2017. Cell-Penetrating Peptides: Design Strategies beyond Primary Structure and Amphipathicity. Molecules. 22(11). In one embodiment, the cell-penetrating peptide is an analog of a cell-penetrating peptide, or SEQ ID NO:36. The cell-penetrating peptide within the gel-forming polypeptide of the present invention, however, can be shorter or longer (e.g., 5-50 or more amino acids in length).

A kallikrein regulator component of a gel-forming polypeptide comprises a homolog, a variant, an analog, a chimera, or a functional fragment of kallikrein regulator, for example SEQ ID NO:37, including without limitation the sequence of SEQ ID NO:37. In another embodiment, the gel-forming kallikrein regulator comprises an amino acid sequence that is about 70%, 75%, 80%, 85%, 90%, or 95% identical to SEQ ID NO:242, or may comprise or consist of SEQ ID NO:242. The term kallikrein regulator, as used herein, may refer to any functional analog which activates or inhibits a kallikrein enzyme (e.g., Ecallantide). In one embodiment, the kallikrein regulator is an analog of a kallikrein activator, inhibitor, or SEQ ID NO:37. The kallikrein regulator within the gel-forming polypeptide of the present invention, however, can be shorter or longer (e.g., 5-50 or more amino acids in length).

A polypeptide antibiotic or antimicrobial peptide component of a gel-forming polypeptide comprises a homolog, a variant, an analog, a chimera, or a functional fragment of a polypeptide antibiotics or antimicrobial peptide, for example SEQ ID NO:68, including without limitation the sequence of SEQ ID NO:68. In another embodiment, the gel-forming polypeptide antibiotics comprises an amino acid sequence that is about 70%, 75%, 80%, 85%, 90%, or 95% identical to SEQ ID NO:243, or may comprise or consist of SEQ ID NO:243. The term polypeptide antibiotics, as used herein, may refer to any functional analog which inhibits the growth or proliferation of a microorganism, bacteria, a fungus, a virus, a tumor, or another pathological agent. In one embodiment, the polypeptide antibiotic is an analog of a polypeptide antibiotics (e.g., temporin A, Gramicidin A, B, C, and D peptides, defensins, esculentin 1-21, cecropins, andropin, moricin, ceratotoxin, melittin, Magainin, dermaseptin, bombinin, brevinin-1, esculentins, buforin II, CAP18, LL37, abaecin, apidaecins, prophenin, indolicidin, actinomycin, bacitracin, colistin, polymyxin B, actinomycin-D, Bacitracin, Boceprevir, Dalbavancin, Daptomycin, Enfuvirtide, Oritavancin, Teicoplanin, Telaprevir, Telavancin, and Vancomycin), or SEQ ID NO:68. The polypeptide antibiotics within the gel-forming polypeptide of the present invention, however, can be shorter or longer (e.g., 5-50 or more amino acids in length).

A complement regulator component of a gel-forming polypeptide comprises a homolog, a chimera, a variant, an analog, a chimera, or a functional fragment of a complement regulator, for example SEQ ID NO:11, including without limitation the sequence of SEQ ID NO:11. In another embodiment, the gel-forming complement regulator comprises an amino acid sequence that is about 70%, 75%, 80%, 85%, 90%, or 95% identical to SEQ ID NO:11 or 244, or may comprise or consist of SEQ ID NO:11 or 244. The term complement regulator, as used herein, may refer to any functional analog (e.g., complement component 5a and compstatin analogs) which activates or inhibits a complement factor or the complement cascade. In one embodiment, the complement regulator is an analog of a complement activator or inhibitor (e.g., compstatin, complement component C5a, C2a, C4b, C3, C3a, C3b, C5b, C6, C7, C8, and C9), or SEQ ID NO:11. The complement regulator within the gel-forming polypeptide of the present invention, however, can be shorter or longer (e.g., 5-50 or more amino acids in length).

A C5aR receptor ligand component of a gel-forming polypeptide comprises a homolog, a variant, a chimera, an analog, a chimera, or a functional fragment of a C5aR receptor ligand, for example SEQ ID NO:11, including without limitation the sequence of SEQ ID NO:11. In another embodiment, the gel-forming C5aR receptor ligand comprises an amino acid sequence that is about 70%, 75%, 80%, 85%, 90%, or 95% identical to SEQ ID NO:11 or 244, or may comprise or consist of SEQ ID NO:11 or 244. The term C5aR receptor ligand, as used herein, may refer to any functional analog (e.g., complement component 5a and compstatin analogs) which activates or inhibits a C5aR receptor. In one embodiment, the C5aR receptor ligand is an analog of a C5aR receptor activator or inhibitor (e.g., compstatin, complement component C5a, C2a, C4b, C3, C3a, C3b, C5b, C6, C7, C8, and C9), or SEQ ID NO:11. The C5aR receptor ligand within the gel-forming polypeptide of the present invention, however, can be shorter or longer (e.g., 5-50 or more amino acids in length). Each possibility

A Copaxone immunomodulator component of a gel-forming polypeptide comprises a homolog, a variant, an analog, a chimera, or a functional fragment of an immunomodulator, for example SEQ ID NO:42, including without limitation the sequence of SEQ ID NO:42. In another embodiment, the gel-forming Copaxone immunomodulator comprises an amino acid sequence that is about 60%, 65%, 70%, 75%, 80%, 85%, 90%, or 95% identical to SEQ ID NO:245, or may comprise or consist of SEQ ID NO:245. The term Copaxone immunomodulator, as used herein, may refer to any functional analog which are composed of the four amino acids found in myelin basic protein, namely glutamic acid, lysine, alanine, and tyrosine and activates or inhibits the glatiramer acetate-mediated signaling pathway (e.g., Copolymer 1, Cop-1 or Copaxone, Glatopa, and M356). In one embodiment, the immunomodulator is an analog of glatiramer acetate (which is a mixture of analogs composed of the four amino acids found in myelin basic protein), or SEQ ID NO:42. The immunomodulator within the gel-forming polypeptide of the present invention, however, can be shorter or longer (e.g., 5-60 or more amino acids in length).

A matrix-modifying protein (or matrikine) component of a gel-forming polypeptide comprises a homolog, a variant, an analog, a chimera, or a functional fragment of a matrix-modifying protein, for example SEQ ID NOS:39, and 44-47, including without limitation the sequence of SEQ ID NOS:39, and 44-47. In another embodiment, the gel-forming matrix-modifying protein comprises an amino acid sequence that is about 70%, 75%, 80%, 85%, 90%, or 95% identical to SEQ ID NO:246, 247, 248, 249, or 250, or may comprise or consist of SEQ ID NO:246, 247, 248, 249, or 250. The term matrix-modifying protein, as used herein, may refer to any functional analog which activates or inhibits a matrix enzyme-mediated signaling pathway. In one embodiment, the matrix-modifying protein is an analog of a modulator of dermal extracellular matrix, collagen modulator, elastin modulator, keratinocytes/epidermal cell modulator, melanogenesis modulator, a structural peptide that act as signal modulators of the extracellular matrix component, as structural peptides, carrier peptides and neurotransmitter function modulators (e.g., YIGSR, Pal-KTTKS, Pal-GHK, GERK, RGD, GQPR, VGVAPG, HFRW, YRSRKYSSVVY, and argireline peptides), or SEQ ID NO:39, or 44-47 (See Pai et al., 2016 Topical peptides as cosmeceuticals. Indian J Dermatol Venereol Leprol. 83:9-18). The matrix-modifying protein within the gel-forming polypeptide of the present invention, however, can be shorter or longer (e.g., 5-40 or more amino acids in length) than the provided sequence.

A thrombopoietin receptor ligand of a gel-forming polypeptide comprises a homolog, a variant, an analog, a chimera, or a functional fragment of a thrombopoietin receptor ligand, for example SEQ ID NO:9, including without limitation the sequence of SEQ ID NO:9. In another embodiment, the gel-forming matrix-modifying protein comprises an amino acid sequence that is about 70%, 75%, 80%, 85%, 90%, or 95% identical to SEQ ID NO:9, or may comprise or consist of SEQ ID NO:9. The term thrombopoietin receptor ligand, as used herein, may refer to any functional analog (e.g., Nplate® (romiplostim)) which activates or inhibits a thrombopoietin receptor. In one embodiment, the thrombopoietin receptor ligand is an analog of thrombopoietin (THPO), megakaryocyte growth and development factor (MGDF), or SEQ ID NO:9. The thrombopoietin receptor ligand within the gel-forming polypeptide of the present invention, however, can be shorter or longer (e.g., 5-50 or more amino acids in length).

An insulin receptor ligand of a gel-forming polypeptide comprises a homolog, a variant, an analog, a chimera, or a functional fragment of a insulin receptor ligand, for example SEQ ID NO:59, including without limitation the sequence of SEQ ID NO:59. In another embodiment, the gel-forming matrix-modifying protein comprises an amino acid sequence that is about 70%, 75%, 80%, 85%, 90%, or 95% identical to SEQ ID NO: 251, or may comprise or consist of SEQ ID NO:251. The term insulin receptor ligand, as used herein, may refer to any functional analog (e.g., insulin degludec, Insulin lispro, Insulin aspart, Insulin glulisine, Insulin detemir, and Insulin glargine) which activates or inhibits an insulin receptor. In one embodiment, the insulin receptor ligand is an analog of insulin, or SEQ ID NO:59. The insulin receptor ligand within the gel-forming polypeptide of the present invention, however, can be shorter or longer (e.g., 5-50 or more amino acids in length for B and A chain, respectively).

A relaxin receptor ligand of a gel-forming polypeptide comprises a homolog, a variant, an analog, a chimera, or a functional fragment of a relaxin receptor ligand, for example SEQ ID NO:60, including without limitation the sequence of SEQ ID NO:60. In another embodiment, the gel-forming matrix-modifying protein comprises an amino acid sequence that is about 70%, 75%, 80%, 85%, 90%, or 95% identical to SEQ ID NO:252, or may comprise or consist of SEQ ID NO:252. The term relaxin receptor ligand, as used herein, may refer to any functional analog which activates or inhibits a relaxin receptor (i.e., LGR7 and LGR8). In one embodiment, the relaxin receptor ligand is an analog of relaxin 1, relaxin 2, relaxin 3, INSL3, INSL7, or SEQ ID NO:60. The relaxin receptor ligand within the gel-forming polypeptide of the present invention, however, can be shorter or longer (e.g., 5-50 or more amino acids in length for B and A chain, respectively).

A parathyroid hormone related peptide (PTHrP) receptor ligand component of a gel-forming polypeptide comprises a homolog, a variant, an analog, a chimera, or a functional fragment of a PTHrP receptor agonist or antagonist, for example SEQ ID NO: 63, including without limitation the sequence of SEQ ID NO: 63. In another embodiment, the gel-forming PTHrP receptor ligand comprises an amino acid sequence that is about 70%, 75%, 80%, 85%, 90%, or 95% identical to SEQ ID NO:258, or may comprise or consist of SEQ ID NO:258. The term PTHrP receptor ligand, as used herein, may refer to any functional analog (e.g., parathyroid hormone, parathyroid hormone-related protein, Teriparatide, and Abaloparatide) which activates or inhibits a PTHrP receptor. In one embodiment, the PTHrP receptor ligand is an analog of a PTHrP receptor agonist, antagonist, PTHrP 1-34, abaloparatide, or SEQ ID NO:63. The PTHrP receptor ligand within the gel-forming polypeptide of the present invention, however, can be shorter or longer (e.g., 5-50 or more amino acids in length).

The sequence of the polypeptide may be altered in various ways known in the art to generate targeted changes in sequence. The polypeptide will usually be substantially similar to the sequences provided herein, i.e. will have greater than 70%, greater than 80%, greater than 90%, greater than 95% sequence identity with the provided sequence. The sequence changes may be substitutions, insertions or deletions. Scanning mutations that systematically introduce alanine, or other residues, may be used to determine key amino acids. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine). Nonconservative substitutions could include any unusual amino acids.

Modifications of interest that do not alter primary sequence include chemical derivatization of polypeptides, e.g., methylation, acetylation, acylation, pegylation, or carboxylation. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes.

In yet other embodiments, the peptide comprises an optical isomer, enantiomer, diastereomer, tautomer, cis-trans isomer, racemate, prodrug or pharmaceutically acceptable salt of a peptide. The peptides may be amidated at the C-termini. The modified peptides optionally contain an acylation modification. Optionally, the number of acylation can be more than one, with one reactive group being preferable.

Peptide formulations also include a mixture of stereoisomers, or each pure or substantially pure isomer. For example, the present compound may optionally have one or more asymmetric centers at a carbon atom containing any one substituent. Therefore, the compound may exist in the form of enantiomer or diastereomer, or a mixture thereof. When the present compound contains a double bond, the present compound may exist in the form of geometric isomerism (cis-compound, trans-compound), and when the present compound contains an unsaturated bond such as carbonyl, then the present compound may exist in the form of a tautomer, and the present compound also includes these isomers or a mixture thereof. The starting compound in the form of a racemic mixture, enantiomer or diastereomer may be used in the processes for preparing the present compound. When the present compound is obtained in the form of a diastereomer or enantiomer, they can be separated by a conventional method such as chromatography or fractional crystallization. In addition, the present compound includes an intramolecular salt, hydrate, solvate or polymorphism thereof.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. For examples, the backbone of the peptide may be cyclized by adding cyclic disulfide bridge or lactam bridge to enhance stability (see Friedler et al. (2000) J. Biol. Chem. 275:23783-23789). Analogs of such polypeptides include those containing residues other than naturally occurring L-amino acids, e.g. D-amino acids or non-naturally occurring synthetic amino acids.

The subject peptides may be prepared by in vitro synthesis, using conventional methods as known in the art. Various commercial synthetic apparatuses are available, for example, automated synthesizers by Applied Biosystems, Inc., Foster City, Calif., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using H PLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99% by weight, in relation to contaminants related to the method of preparation of the product and its purification. Usually, the percentages will be based upon total protein.

### Compositions and Uses of Gel-Forming Polypeptides

The present invention is directed to the generation and use of self-assembled gel-forming polypeptides. In some embodiments the polypeptides are derived from secreted human peptide hormones, human peptide analogs, and polypeptides with gel-forming-enhancing motifs. The gel-forming polypeptides may be used in a method of treating a patient with the native or engineered gel-forming polypeptide, the method comprising administering a therapeutic composition comprising an effective dose of a gel-forming polypeptide, alone or in combination with an additional therapeutic agent, to an individual in need thereof.

Provided a series of peptide sequences comprising or mimicking natural polypeptide therapeutics, but with a gel-forming capability. The compositions and improved methods for generating self-assembling polypeptides may comprise dissolving such polypeptides at suitable concentrations or conjugating them with a gel-forming-enhancing motif. Biocompatible and biodegradable polypeptide gels are useful for delivering a therapeutic agent, either as a carrier protein, wherein the therapeutic agent is encapsulated/associated within a gel-forming polypeptide carrier; or as a therapeutic agent itself.

The use of this technique allows: (1) the generation of gel-forming therapeutic analogs for which the wild-type or known analog normally do not form gel by itself, (2) the formation of large order nonimmunogenic polypeptide gel nanostructures to encapsulate therapeutic agents for sustained release by decreasing solubility and providing surface barrier, and (3) the reduction of degradation or clearance of polypeptide therapeutic agents in vivo as well as (4) the use of gel-forming native or wild-type hormonal/therapeutic therapeutics in a formulation that has an extended resident time in vivo. The reversable phase transition of noncovalently associated monomers in polypeptide gel nanostructure allows the sustained delivery of therapeutic agents in a bioactive form for an extended period of time when compared with an injection of an aqueous solution or a crystal particle suspension formulation. The resulting liquid or semisolid gels generally exhibit minimal immunogenic response and readily degradable.

In some embodiments, aspects of the invention further include one or more bioactive agents adsorbed or absorbed within the hydrogels and where the hydrogel is configured to deliver the one or more bioactive agent to a site of administration, such as by implanting the subject hydrogel, coating an implant with the hydrogel, ingesting the hydrogel. The amount of bioactive agent incorporated will depend on the duration of delivery, site of application as well as the condition being treated. In some embodiments, the amount of bioactive agent incorporated into the subject hydrogels is 0.0001 μg or greater, such as 0.001 μg or greater, such as 0.01 μg or greater, such as 0.1 μg or greater, such as 1 μg or greater, such as 10 μg or greater, such as 25 μg or greater, such as 50 μg or greater, such as 100 μg or greater such as 500 μg or greater, such as 1000 μg or greater such as 5000 μg or greater and including 10,000 μg or greater. Where the bioactive agent is incorporated into the hydrogels as a liquid, the concentration of bioactive agent may be 0.0001 μg/mL or greater, such as 0.001 μg/mL or greater, such as 0.01 μg/mL or greater, such as 0.1 μg/mL or greater, such as 0.5 μg/mL or greater, such as 1 μg/mL or greater, such as 2 μg/mL or greater, such as 5 μg/mL or greater, such as 10 μg/mL or greater, such as 25 μg/mL or greater, such as 50 μg/mL or greater, such as 100 μg/mL or greater such as 500 μg/mL or greater, such as 1000 μg/mL or greater such as 5000 μg/mL or greater and including 10,000 μg/mL or greater.

The effective dose of a therapeutic polypeptide delivered as a gel formulation to a human patient may be from about 1.0 μg/kg weight, 2.5 μg/kg weight, 5.0 μg/kg weight, 10.0 μg/kg weight, 25.0 μg/kg weight, 50.0 μg/kg weight, 75.0 μg/kg weight, 0.1 mg/kg weight, 0.5 mg/kg weight, 1.0 mg/kg weight, 2.5 mg/kg weight, 5.0 mg/kg weight, 7.5 mg/kg weight, 10.0 mg/kg weight, 25.0 mg/kg weight, 50.0 mg/kg weight, 75.0 mg/kg weight, 1100 mg/kg weight, 250 mg/kg weight, 500 mg/kg weight, 750 mg/kg weight, 1 g/kg weight, 2 g/kg weight, 5 g/kg weight, 10 g/kg weight, 25 g/kg weight up to about 50 g/kg weight, or more, or any range of doses with these parameters.

The effective dose may be maintained for a period of time sufficient to treat the condition. Depending on the structure of the specific hydrogel employed, the release of the one or more bioactive agents from the hydrogel matrix may vary. For example, hydrogels of the present invention may be configured to provide a sustained release. The gel may be designed to release a therapeutic dose of the polypeptide for 1, 2, 3, 4, 5, 6, 7, 8, 9, 10, 11, 12, 13, 14 or more day, e.g. 1 week, 2 weeks, 3 weeks, 4 weeks or more.

By “sustained release” is meant that the hydrogel is structured to provide for continuous delivery of one or more bioactive agents over the entire time hydrogel is maintained in contact with the site of administration, such as over the course of 1 day or longer, such as 2 days or longer, such as 5 days or longer, such as 10 days or longer, such as 15 days or longer, such as 30 days or longer and including 100 days or longer.

Aspects of the invention also include methods for treating a subject by applying one or more gel of the invention to the subject. In some embodiments, methods include applying a hydrogel solution or suspension to the subject and maintaining the hydrogel in contact with the subject in a manner sufficient to treat the subject. As discussed above, hydrogels of interest may be applied to any suitable application site in need of treatment, including by not limited to the skin, bones, heart, liver, kidneys, bladder, trachea, lungs, tumor tissues, in the mouth such as buccally and sublingually and within the nose, throat, ears, uterus, and bladder etc.

In certain embodiments, methods may include applying one or more hydrogels and maintaining the hydrogel in contact with the subject in a manner sufficient to deliver a target dosage of bioactive agent, such as for example as characterized by total bioactive agent exposure or by average daily bioactive agent exposure. For example, the target dosage of bioactive agent delivered by subject methods may be 0.01 mg/day or greater, such as 0.04 mg/day or greater, such as 0.5 mg/day or greater over a 4 week dosage interval, such as 1.0 mg/day or greater, such as 2 mg/day or greater, such as 5 mg/day or greater and including 10 mg/day over a 4 week dosage interval.

As discussed herein, gel-forming-enhancing motifs are identified, which improve the generation of gellable molecules. The minimal structural change associated with conjugation of a gel-enhancing motif has minimal effects on the volume of distribution characteristics of the therapeutic agents discussed herein. A minimal effect on volume of distribution of said modification is beneficial, as it allows the therapeutic agent to distribute in vivo with pharmacokinetics similar to a wild-type counterpart polypeptide. This property is preferred in many situations because the use of other half-life extension techniques such as the fusion to a large molecule (e.g., fusion to an IgG, albumin, or PEG) would drastically increase the molecular mass and reduce the volume of distribution of the said therapeutics, thereby preventing them from reaching many intended targets outside of the general circulation.

The discovery that dissolving select polypeptide compounds at suitable concentrations of (i.e., below, at, or above 11% w/w) in aqueous solution results in stable gel formulations is unexpected. The selection of 11% w/w as an initial testing point is based on the understanding that serum protein levels is approximately 9-10% w/w. Such a polypeptide is able to self-assemble into a liquid gel or semisolid gel nanostructure when it reaches a critical aggregation concentration. Formation of liquid gel or semisolid gel by the native or modified polypeptides is specifically contemplated.

These discoveries allow a slow release formulation with a high loading capacity of therapeutic polypeptides and other therapeutics when compared with the conventional compositions and formulations. All these advantages can be obtained by using a suitable formulation or engineering a polypeptide to provide a gel nanostructure formation at a concentration where the wild-type counterpart polypeptide remains as an aqueous solution, partially soluble or insoluble known to a person skilled in the art. By entrapping a therapeutic agent inside a higher-ordered gel nanostructure formed by self-assembling polypeptides, the gel formulation allows for prolonged delivery of therapeutic agents, thereby increasing patient adherence, drug delivery efficiency and efficacy, and reducing adverse effects. In addition, this approach could mitigate non-specific interactions and protects against enzymatic degradation and improve solubility. Furthermore, the gel-forming therapeutics represents discrete molecule, not a complex, and can have deepter distribution into tissues when compared to therapeutics that are conjugated or complexed with large carrier molecules.

These and other advantages are achieved based on the phenomenon that noncovalently associated polypeptide gels undergo phase transition between aqueous solution and gel nanostructure in a dosage-, time- and other environment factor-dependent manner. A gel nanostructure generally dissociates into monomeric molecules in an aqueous solution as the molecules in the peripheral surface of the gel gradually diffuse into the surrounding solution.

In a preferred embodiment, all percentages mentioned in the present invention are weight/weight (w/w) percentages.

### EXPERIMENTAL

Based on the observation that, amylin, a type B GPCR ligand, naturally forms solid amyloid fiber in vivo; and that the related adrenomedullin peptide CGRP forms liquid gel nanostructure at select concentrations, we hypothesized that the amylin/CGRP/adrenomedullin/adrenomedullin 2 family peptide is endowed with the propensity to form a gel nanostructure when properly formulated or engineered. This hypothesis was further supported by our observation that select acylated adrenomedullin agonists and antagonists form a gel nanostructure in aqueous solution naturally and that engineering of select polypeptides with a hydrophobic tail, head, or side chain in the presence of a gel-forming-enhancing motif can promote gel formation of these polypeptides. In addition, we explored the gel-forming propensity of native and artificial therapeutic peptides in aqueous solution. Determination of Compound Gel-forming Ability in Water: To determine the solubility of polypeptides at room temperature, 1, 2, 3, 4, 5, 20, or 100 mg of a selected peptide is weighed and deposited into a clear plastic or glass vial and an aliquot of de-ionized water was then added to the vial. The procedure takes place in a room which is maintained at approximately 22-25° C. Soluble peptide samples dissolve instantly and a clear solution is observed. The solution is observed to be clear and fluid thus indicating that the solubility of the peptide is greater than at the appropriate concentration (w/w). Insoluble peptides sample retained the particles or paste-like characteristics after 20 min of reaction. In addition to visual inspection, we performed a tube tapping assay and a tilted tube assay to differentiate the gelling status of peptide solutions.

To determine the solubility of polypeptides at room temperature, 1, 2, 3, 4, 5, 20, or 100 mg of a selected peptide is weighed and deposited into a clear plastic vial and an aliquot of de-ionized water was then added to the vial. The procedure takes place in a room which is maintained at approximately 22-25° C. Polypeptide sample which is considered soluble dissolves instantly in water and stays as a clear solution without obvious macroscopic change in viscosity at 20 min after mixing with water. This type of observation indicates that the solubility of the peptide is high and the peptide does not form gel nanostructure. If macroscopic particles of peptides remain in the solution at 20 min after mixing with water, the peptide is considered insoluble or precipitated at the given concentration. In this case, the quality of peptides is further judged by two other criteria: (1) mobility following a tapping of the tube (tube tapping assay) and (2) mobility flowing tilting the tube by 900 (tilted tube assay). In the first assay, the tube is tapped with the finger for 10 times, and the number of times that the body of the solution changes position is recorded. An aqueous solution such as water will have a score of 9 or 10 in this assay; meaning that the body of the liquid moves every time when one taps the tube. Likewise, an insoluble or precipitated peptide in solution will exhibit the same score; that is a score of 9 or 10 and macroscopic change in positions of the insoluble peptide particles or precipitates can be observed with each tapping. By contrast, the gel-forming peptides dissolve instantly or gradually in the aqueous solution and exhibit high viscosity at 20 min after mixing with water. Those with moderate viscosity form liquid gel that changes the conformation slowly when the container tube is tapped with a finger or tilted. To differentiate the liquid gels from semisolid gels, we tested the mobility of the solution based on the tube tapping assay and the tilted tube assay. Selected peptide solutions exhibited extremely high viscosity and formed semisolid gels that do not flow as a liquid when the container is tapped with a finger or tilted. In the tube tapping assay, if the gel has a score of 0-4 (i.e., macroscopic change in the position of the body of gel after tapping was observed in 0-4 times out of 10 separate tapping tests), it is considered a semisolid gel. If macroscopic change in the position of the body of gel was observed 5-9 time out of 10 separate tapping tests, the gel is considered a liquid gel. The high viscosity of a liquid gel is further confirmed using the tilted tube assay. In this assay, the plastic tube that contains the solution or gel was tilted 900, and the movement of the solution or the gel mass is observed under the light, if the liquid body moves to the bottom of the tube in less than 15 second, the peptide is considered a liquid solution. If the liquid or gel body takes more than 15 seconds to flow to the bottom of the tilted tube, the resulting aqueous mass is considered a liquid gel.

To study the bioactivities of conjugated and mutated peptides, we employed in vitro receptor-activation assays with cultured cells expressing recombinant melanocortin receptor 1 (MC1R), melanocortin receptor 4 (MC4R), CRH receptor 2 (CRHR2), PTH receptor 1 (PTHR1), or kappa opioid receptor (OPRK1). The agonistic activities toward MC1R, MC4R, CRHR2, PTHR1, and OPRK1 were assyed with an MC1R cAMP assay, an MC4R cAMP assay, a CRHR2 cAMP assay, a PTHR1 cAMP assay and an OPRK1 cAMP assay from Discoverx Inc. (Fremont, Calif.), respectively. The antagonistic activities toward bradykinin receptor 2 (BDKRB2) and GnRH receptor (GnRHR or LHRHR) were assayed with a BDKRB2 arrestin assay and a GnRHR calcium flux assay from Discoverx, respectively.

For the analysis of Gs-coupled cAMP production, cAMP Hunter cell lines were expanded from freezer stocks, and cells were seeded in a total volume of 20 μL into white walled, 384-well microplates and incubated at 37 C for the appropriate time prior to testing. The cAMP modulation was determined using the DiscoverX HitHunter cAMP XS+ assay. For agonistic activity determination, cells were incubated with sample to induce response. Media was aspirated from cells and replaced with 15 μL 2:1 HBSS/10 mM Hepes: cAMP XS+ Ab reagent. Intermediate dilution of sample stocks was performed to generate 4× sample in assay buffer, and 5 μL of 4× sample was added to cells and incubated at 37 C or room temperature for 30 or 60 minutes. Vehicle concentration was 1%. The compound activity was analyzed using a CBIS data analysis suite (ChemInnovation, CA). For Gs-coupled agonistic assays, percentage activity is calculated using the following formula: % Activity=100%×(mean RLU of test sample−mean RLU of vehicle control)/(mean RLU of MAX control−mean RLU of vehicle control).

For the Arrestin assays, PathHunter cell lines (Discoverx Inc.) were expanded from freezer stocks, and seeded in a total volume of 20 μL into white walled, 384-well microplates and incubated at 37 C for the appropriate time prior to testing. For agonist determination, cells were incubated with sample to induce response. Intermediate dilution of sample stocks was performed to generate 5× sample in assay buffer, and 5 μL of 5× sample was added to cells and incubated at 37 C or room temperature for 90 to 180 minutes. Vehicle concentration was 1%. For antagonistic activity determination, cells were pre-incubated with antagonist followed by agonist challenge at the EC80 concentration. Intermediate dilution of sample stocks was performed to generate 5× sample in assay buffer, and 5 μL of 5× sample was added to cells and incubated at 37° C. or room temperature for 30 minutes. Vehicle concentration was 1%. Then, 5 μL of 6×EC80 agonist in assay buffer was added to the cells and incubated at 37 C or room temperature for 90 or 180 minutes.

Assay signal was generated through a single addition of 12.5 or 15 μL (50% v/v) of PathHunter Detection reagent cocktail, followed by an one hour incubation at room temperature. The microplates were read following signal generation with a PerkinElmer Envision™ instrument for chemiluminescent signal detection. The compound activity was analyzed using a CBIS data analysis suite (ChemInnovation, CA). For agonist mode assays, percentage activity was calculated using the following formula: % Activity=100%×(mean RLU of test sample−mean RLU of vehicle control)/(mean MAX control ligand−mean RLU of vehicle control). For antagonistic activity assays, percentage inhibition was calculated using the following formula: % Inhibition=100%×(1−(mean RLU of test sample−mean RLU of vehicle control)/(mean RLU of EC80 control−mean RLU of vehicle control)).

For the analysis of calcium flux, cell lines were expanded from freezer stocks and seeded in a total volume of 20 μL into black-walled, clear-bottom, Poly-D-lysine coated 384-well microplates and incubated at 37 C for the appropriate time prior to testing. Assays were performed in 1× Dye Loading Buffer consisting of 1× Dye, 1× Additive A and 2.5 mM Probenecid in HBSS/20 mM Hepes. Cells were loaded with dye prior to testing. Media was aspirated from cells and replaced with 20 μL Dye Loading Buffer. Cells were incubated for 30-60 minutes at 37 C. For agonist determination, cells were incubated with sample to induce response. After dye loading, cells were removed from the incubator and 10 μL HBSS/20 mM Hepes was added. Vehicle was included in the buffer when performing agonist dose curves to define the EC80 for antagonist assays. Cells were incubated for 30 minutes at room temperature in the dark to equilibrate plate temperature. Intermediate dilution of sample stocks was performed to generate 4× sample in assay buffer. Compound agonist activity was measured on a FLIPR Tetra (MDS), and calcium mobilization was monitored for 2 minutes and 10 μL 4× sample in HBSS/20 mM Hepes was added to the cells 5 seconds into the assay.

For antagonist determination, cells were pre-incubated with sample followed by agonist challenge at the EC80 concentration. Intermediate dilution of sample stocks was performed to generate 3× sample in assay buffer. After dye loading, cells were removed from the incubator and 10 μL 3× sample was added. Cells were incubated for 30 minutes at room temperature in the dark to equilibrate plate temperature. Vehicle concentration was 1%. Compound antagonistic activity was measured on a FLIPR Tetra (MDS), and calcium mobilization was monitored for 2 minutes and 10 μL EC80 agonist in HBSS/20 mM Hepes was added to the cells 5 seconds into the assay. The compound activity was analyzed using a CBIS data analysis suite (ChemInnovation, CA), and percentage activity is calculated using the following formula: % Activity=100%×(mean RFU of test sample−mean RFU of vehicle control)/(mean MAX RFU control ligand−mean RFU of vehicle control). For antagonist assays, percentage inhibition is calculated using the following formula: % Inhibition=100%×(1−(mean RFU of test sample−mean RFU of vehicle control)/(mean RFU of EC80 control−mean RFU of vehicle control)).

For the analysis of release of select molecules from gels made of gel-forming peptides, we performed release assays using semipermeable chambers such as the Centricon® Centrifugal Filter Units. In these experiments, FITC-labeled small molecule, peptide, or nucleic acid were dissolved in solutions with a low concentration of a gel-forming or a nongel-forming peptide, and loaded onto the upper chamber of Centricon® Centrifugal Filter Units with a 10 kDa or 20 kDa filter membranes. The columns were centrifuged at 2000 or 3000 rpm for 20 min, and the amount of FITC-labeled molecules in the lower chamber was quantified by a fluorometer.

As shown in Table 1, we have tested a wide variety of peptides for the ability to form stable gel by dissolving (or attempting to dissolve) them in water at room temperature. The peptides that form liquid gel or semisolid gel remained stable under the test conditions. These examples demonstrate the effect, or lack of effect, of various modifications on the ability of compositions of the invention to form liquid, solid, or semisolid gel.

The gel-forming capability of a variety of secreted and therapeutic polypeptides or their analogs was determined, shown in Table 1. All peptides were produced by solid phase synthesis, and their ability to form semisolid or liquid gel was analyzed at room temperature. To allow systematic analysis of the gel-forming ability, the assay was first conducted at a peptide concentration of 11% w/w in aqueous solution. The peptides analyzed included functional regulators of cell surface receptors, enzymes, complement factors, antimicrobial peptides, immunomodulators, therapeutic peptides, cell-penetrating peptide, antigens, matrikines, and analogs of CGRP, adrenomedullin and adenomedullin 2 (or intermedin). At 11% w/w in an aqueous excipient, adrenomedullin 1-52, Pramlintide (an amylin analog), oxytocin, kisspeptin, a kappa receptor agonist, Pralmorelin, a thrombopoietin analog (i.e., Romiplostim analog), urocortin 3, a bombesin receptor antagonist, an acylated ADM2-52, and compstatin (SEQ ID NOS: 2, 4-11, 61, and 274) consistently formed semisolid clear or opaque gel in aqueous solution. Likewise, an adrenomedullin analog (i.e., ADE43), CGRP, Teduglutide (a GLP-2 analog), thymosin alpha-1, GLP-1, gamma-MSH, an acylated thymosin beta 4 analog, and a GnRH analog (SEQ ID NOS:1, 3, 12-15, 64, and 263) consistently formed a liquid gel at 11% w/w. On the other hand, a variety of other functional polypeptides (SEQ ID NOS:16-47) either remain as clear liquid solution or form insoluble precipitates at the select concentration after 20 minutes of reaction. Polypeptides that remains as clear aqueous solution or as insoluble precipitates lack the ability to form a gel nanostructure. Abbreviations used in the Table include SS for semisolid gel, LG for liquid gel.

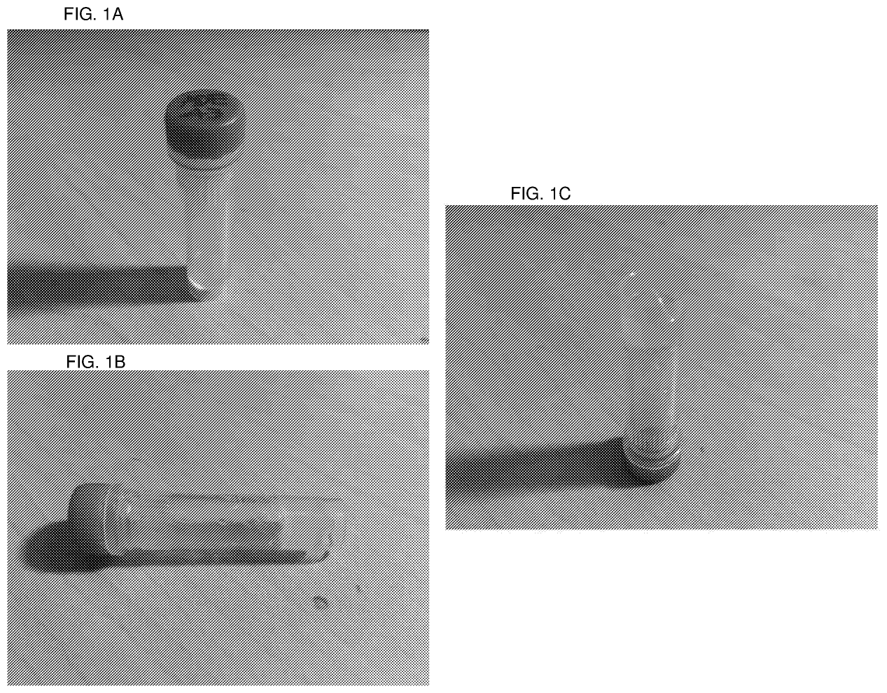

A graphic representation of semisolid gel formed by SEQ ID NO: 1 peptide is shown in FIG. 1. The semisolid gel retains the position when the holding tube is positioned upright (A), tilted 90° (B) or inverted (C).

TABLE 1
The identification of secreted polypeptides that form gel by itself in aqueous
solution.

| | | | |
|---|---|---|---|
| | | | Physical |
| SEQ | | | property |
| ID | | | at |
| NO | Identity | Sequence | 11% w/w |

Functional wild-type peptides or modified analogs

| | | | |
|---|---|---|---|
| 1 | ADE43 | Ace- | LG |
| | | K(PAL)FGCRFGTCTVQKLAHQIYQFTDKDKDNVAPRSKI | |
| | | SPQGY-NH2 | |
| 2 | Adrenomedullin 1-52 | YRQSMNNFQGLRSFGCRFGTCTVQKLAHQIYQFTDKDK | SS |
| | | DNVAPRSKISPQGY-NH2 | |
| 3 | CGRP | acdtatcythrlagllsrsggwknnfvptnvgskaf-NH2 | LG |
| 4 | Pramlintide (amylin | KCNTATCATQRLANFLVHSSNNFGPILPPTNVGSNTY- | SS |
| | analog) | NH2 | |
| 5 | Oxytocin | CYIQNCPLG-NH2 | SS |
| 6 | Kisspeptin | YNWNSFGLRF-NH2 | SS |
| 7 | Kappa receptor | (D-Phe)-(D-Phe)-Trp-(D-Arg) | SS |
| | agonist | | |
| 8 | Pralmorelin | (D-Ala)-(D-(β-naphthyl))-AAW-(D-Phe)-Lys-NH2 | SS |
| 9 | Romiplostim | IEGPTLRQWLAARA | SS |
| 10 | Urocortin 3 | FTLSLDVPTNIMNLLFNIAKAKNLRAQAAANAHLMAQI- | SS |
| | | NH2 | |
| 11 | Compstatin | ICVVQDWGHHRCT-NH2 | SS |
| 12 | Teduglutide (GLP-2 | HGDGSFSDEMNTILDNLAARDFINWLIQTKITD-OH | LG |
| | analog) | | |
| 13 | Thymosin alpha 1 | SDAAVDTSSEITTKDLKEKKEVVEEAEN-NH2 | LG |
| 14 | GLP-1 | HAEGTFTSDVSSYLEGQAAKEFIAWLVKGR-NH2 | LG |
| 15 | GnRH | (G1p)HWSY-(D-2Nal)-LRPG-NH2 | LG |
| 16 | Angiotensin 11 | DRVYIHPF-NH2 | AS/IS |
| 17 | Vasopressin | CYFQNCPRG-NH2 | AS/IS |
| 18 | Apelin | (Glp)RPRLSHKGP(Nle)PF-NH2 | AS/IS |
| 19 | Neurotensin | (Glp)LYENKPRRPYIL | AS/IS |
| 20 | Bombesin | (D-Tyr)QWAV(β-Ala)HF(Nle)-NH2 | AS/IS |
| 21 | Deltorphin | Y-(D-Met)-FHLMD-NH2 | AS/IS |
| 22 | Enkephalin | Y-(D-Ala)-GF(D-Leu)-NH2 | AS/IS |
| 23 | Substance P | RPKPQQFFGLM-NH2 | AS/IS |
| 24 | Saralasin | Sar-RVYVHPA-NH2 | AS/IS |
| 25 | Parathyroid hormone | SVSEIQLMHNLGKHLNSMERVEWLRKKLQDVHNF | AS/IS |
| 26 | Exenatide 4 | HGEGTFTSDLSKQMEEEAVRLFIEWLKNGGPSSGAPPP | AS/IS |
| | | S-NH2 | |
| 27 | GnRH antagonist | (D-Nal)-(D-Cpa)-(D-Pal)-SY-(D-Cit)-LRP-(D-Ala)- | AS/IS |
| | | NH2 | |
| 28 | Calcitonin | CSNLSTCVLGKLSQELHKLQTYPRTNTGSGTP-NH2 | AS/IS |
| 29 | Melanotan I | SYS-Nle-EH-(D-Phe)-RWGKPV-NH2 | AS/IS |
| 30 | PYY3-36 | AKPEAPGEDASPEELSRYYASLRHYLNLVTRQRY-NH2 | AS/IS |
| 31 | Urocortin 2 | IVLSLDVPIGLLQILLEQARARAAREQATTNARILARVGHC- | AS/IS |
| | | NH2 | |
| 32 | HOE140 (a BKR2 | (D-Arg)-Arg-Pro-Hyp-Gly-Thi-Ser-(D-Tic)-Oic-Arg | AS/IS |
| | antagonist) | | |
| 33 | ACTH 1-24 | SYSMEHFRWGKPVGKKRRPVKVYP-OH | AS/IS |
| 34 | Setmelanotide | RC-(D-Ala)-H-(D-Phe)-RWC-NH2. | AS/IS |
| 35 | Sermorelin | YADAIFTNSYRKVLGQLSARKLLQDIMSR-NH2 | AS/IS |
| 36 | TAT peptide (a cell | GRKKRRQRRRPQ | AS/IS |
| | permeability peptide) | | |
| 37 | A kallikrein inhibitor | PFRSVQ-NH2 | AS/IS |
| 38 | Fuzeon | YTSLIHSLIEESQNQQEKNEQELLELDKWASLWNWF- | AS/IS |
| | | NH2 | |
| 39 | Matrix modifying | YIGSR-NH2 | AS/IS |
| | peptide 1 | | |
| 40 | Guanylin | PGTCEICAYAACTGC | AS/IS |
| 41 | Atria atriuretic | SLRRSSCFGGRMDRIGAQSGLGCNSFRY-NH2 | AS/IS |
| | peptide (ANP) | | |
| 42 | Copaxone | EAYKAAEKAYAAKEAAKEAAKAKAEKKAAYAKAKAAKY | AS/IS |
| | | EKKAKKAAAEYKKK | |
| 43 | Matrix modifying | KTTKS-NH2 | AS/IS |
| | peptide 2 | | |
| 44 | RGD peptide | RGD-NH2 | AS/IS |
| 45 | Matrix modifying | VGVAPG-NH2 | AS/IS |
| | peptide 7 | | |
| 46 | Matrix modifying | YRSRKYSSWY-NH2 | AS/IS |
| | peptide 8 | | |
| 47 | An acetyl hexapeptide- | Ac-EEMQRR-NH2 | AS/IS |
| | 3 matrix modifying | | |
| | peptide | | |
| 61 | Bombesin receptor | (D-Phe)-QWAVGHL-(4-CI)Phe-NH2 | SS |
| | antagonist | | |
| 64 | Gamma-MSH | YVMGHFRWDRF-NH2 | LG |
| 263 | Thymosin beta 4 | Pal- | LG |
| | analog | HSDKPDMAEIEKFDKSKLKKTETQEKNPLPSKETIEQEK | |
| | | QAGES-NH2 | |
| 274 | ADM51 analog | Pal- | SS |
| | | RQSMNNFQGLRSFGCRFGTCTVQKLAHQIYQFTDKDK | |
| | | DNVAPRSKISPQGY-NH2 | |

Modified and chimeric polypeptides with adrenomedullin and/or
adrenomedullin 2 sequences

| | | | |
|---|---|---|---|
| 48 | Adrenomedullin | miniPEG- | SS |
| | analog | K(PAL)GCRFGTCTVQKLAHQIYQFTDKDVAPRSKISP(D- | |
| | | Gln)GY-NH₂ | |
| 49 | ADM/ADM2 chimeric | miniPEG- | LG |
| | analog | TK(Pal)KTLRTGCRFGTCTVQKLAHQIYQFTDKDKDNVA | |
| | | PVDPSSPHSY-NH2 | |
| 50 | ADM/ADM2 chimeric | Pal- | LG |
| | analog | CRFGTCTVQKLAHQIYHiQFTDKDKDNSAPVDPSSPHSY- | |
| | | amidated | |
| 51 | ADM/ADM2 chimeric | miniPEG- | LG |
| | analog | K(PAL)GCRFGTCTVQKLAHQIYQFTDKDKDSAPVDPSS | |
| | | PHSY-amidated | |
| 52 | ADM/ADM2 chimeric | miniPEG- | LG |
| | analog | K(PAL)GCRFGTCTVQKLAHQIYQFTDKDKSAPVDPSSP | |
| | | HSY-amidated | |
| 53 | ADM/ADM2 chimeric | miniPEG- | LG |
| | analog | K(PAL)GCRFGTCTVQKLAHQIYQFTDKDSAPVDPSSPH | |
| | | SY-amidated | |
| 54 | Adrenomedullin | miniPEG- | LG |
| | analog | K(PAL)FGCRFGTCTVQKLAHQIYQFTDKDKDNVAPRSKI | |
| | | SPQGY-amidated | |
| 55 | ADM/ADM2 chimeric | miniPEG- | LG |
| | analog | K(PAL)GCRFGTCTVQKLAHQIYQFTDKDKDNSAPVDPS | |
| | | SPHSY-amidated | |
| 56 | ADM/ADM2 chimeric | Pal- | LG |
| | analog | KGCRFGTCTVQKLAHQIYQFTDKDSAPVDPSSPHSY- | |
| | | NH2 | |
| 57 | Adrenomedullin | miniPEG-K(PAL)GCRFGTC(D- | LG |
| | analog | Thr)VQKLAHQIYQFTDKDVAPRSKISPQGY-NH₂ | |
| 58 | Adrenomedullin | miniPEG- | LG |
| | analog | K(PAL)GCRFGTCTVQKLAHQIYQFTDKDVAPR(D- | |
| | | Ser)KISPQGY-NH₂ | |

Studies of the adrenomedullin and adrenomedullin 2 analogs (SEQ ID NOS:48-58 [Table 1], 274-275 [Table 3]) showed that select analogs form a liquid gel at 11% w/w, and that minor sequence modification can lead to dramatic changes in the gel-forming capability. Whereas the parental peptide, the wild-type truncated adrenomedullin analog (SEQ ID NO: 1) forms liquid gel at 11% w/w, a D-amino acid substitution at select residue leads to the formation of a semisolid gel by SEQ ID NOS:48. On the other hand, palmitoylation modification in SEQ ID NO:274 allowed the analog to form semisolid gel at 6%, which is superior to the wild-type peptides and other adrenomedullin analogs. Other modifications as shown in the peptides of SEQ ID NOS:49-58 allowed these analogs to retain the ability to form liquid gel at 11% w/w. Although gel-forming capability is regulated by many physical and chemical factors, gel-forming peptides, such as adrenomedullin and adrenomedullin 2 may contain sequence motifs that promote gel formation, and changes in residues or side chains can alter the gel-forming capability of these peptides.

These data also indicated that the gel-forming peptides such as adrenomedullin, Pramlintide (an amylin analog), oxytocin, kisspeptin, a kappa opioid receptor agonist, Pralmorelin, a Romiplostim analog, urocortin 3, compstatin, CGRP, Teduglutide (a GLP-2 analog), thymosin alpha 1, GLP-1, a GnRH, a bombesin receptor antagonist, gamma-MSH, thymosin beta 4, and an adrenomedullin analog (SEQ ID NOS:1-15, 61, 64, 263, and 274) can be delivered using a self-assembling gel formulation, i.e. optionally in the absence of additional gel-forming agents. However, these gel-forming peptides could contain gel-forming-enhancing motifs that promote self-assemble gel formation.

Based on the finding that CGRP/adrenomedullin family peptides form liquid or semisolid gel in aqueous solution, we hypothesized that these self-assembling gel-forming polypeptides might contain gel-forming-enhancing motifs that increase the gel-forming capability of polypeptides that normally do not form gel nanostructure. We then tested the ability to form semisolid or liquid gel by a series of analogs of the calcitonin/amylin/CGRP/adrenomedullin/adrenomedullin2 family peptides based on visual examination, a tube tapping assay and a tilted tube assay at room temperature. To allow systematic analysis of the gel-forming ability, the assay was performed at 11% w/w. As shown in Table 2, some of the modified and chimeric adrenomedullin/adrenomedullin 2 analogs, similar to wild-type adrenomedullin (SEQ ID NOS:106-114 and 116-123) form semisolid or liquid gel at 11% w/w. By contrast, select modifications (e.g., truncation and substitution; SEQ ID NOS:101-105 and 115) resulted in polypeptides that stay as an aqueous solution or are insoluble at the same concentration.

Among these peptides, we found that analogs as short as 3-amino-acid in length (SEQ ID NOS: 120-121) are able to form semisolid gels at 11% w/w or lower. Although a single amino acid change in an adrenomedullin/adrenomedullin 2 peptides can lead to the generation of analogs that stays as aqueous solution or insoluble, or form liquid or semisolid gels, these data suggested that ADM/ADM2 derived peptides have a propensity to form liquid or semilsolid gels. This observation also supports the hypothesis that gel-forming-enhancing motifs derived from self-assemble gel-forming peptides may be used to render non-gel-forming peptides into self-assemble gel-forming peptides. While many truncated adrenomedullin/adrenomedullin 2 analogs form semisolid or liquid gels (SEQ ID NOS: 116-124 and 126-131), polypeptides composed of sequences from corresponding regions in CGRP or amylin (SEQ ID NOS: 132-134) remain as aqueous solution or insoluble precipitates at the same concentration. Nonetheless, modification of the CGRP sequences with a fragment of adrenomedullin sequences can lead the generation of analogs that form aqueous solution, liquid gel, or semisolid gel (SEQ ID NOS: 139-140). At 11% w/w, the gel-forming control peptides that composed of repeat sequences (SEQ ID NOS: 141-142), as reported previously, also form semisolid gel in aqueous solution.

TABLE 2
The identification of adrenomedullin, CGRP and
IMD analogs that form gel by itself in aqueous
solution.

| | | Physical |
|---|---|---|
| | | property |
| SEQ | | at |
| ID | Sequence | 11% w/w |
| 101 | Ace-TVQKLAHQIYQFTDKDKDNVAPRSKISPQGY- | AS/IS |
| | NH2 | |
| 102 | Pal-VQKLAHQIYQFTDKDVAPRSKISPQGY-NH2 | AS/IS |
| 103 | Pal-QKLAHQIYQFTDKDVAPRSKISPQGY-NH2 | AS/IS |
| 104 | Pal-KVQNLSAPVDPSSPHSY-NH2 | AS/IS |
| 105 | Pal-KVQKLAHQSAPVDPSSPHSY-NH2 | AS/IS |
| 106 | MiniPEG-K(Pal)VQKLAHQIYSAPVDPSSPHSY- | LG |
| | NH2 | |
| 107 | Pal-KVQKLAHQIYQFTDKDVAPRSKISPQGY-NH2 | LG |
| 108 | Pal-KVQKLAHQIYQFTDKDSAPVDPSSPHSY-NH2 | LG |
| 109 | Pal-KVQKLSAPVDPSSPHSY-NH₂ | LG |
| 110 | Pal-KVSAPVDPSSPHSY-NH₂ | LG |
| 111 | Pal-VQKLAPVDPSSPHSY-NH₂ | LG |
| 112 | Pal-VQKLVDPSSPHSY-NH₂ | SS |
| 113 | Pal-VQKLDPSSPHSY-NH₂ | LG |
| 114 | Pal-VQKLPSSPHSY-NH₂ | LG |
| 115 | Ace-KVQKLSAPVDPSSPHSY-NH₂ | AS/IS |
| 116 | Pal-VQKPSSPHSY-NH₂ | LG |
| 117 | Pal-VQKLPHSY-NH₂ | SS |
| 118 | Pal-KPSSPHSY-NH₂ | SS |
| 119 | Pal-SSPHSY-NH₂ | SS |
| 120 | Pal-HSY-NH₂ | SS |
| 121 | Pal-KSY-NH₂ | SS |
| 122 | (Lauric acid)-SSPHSY-NH₂ | SS |
| 123 | (Myristic acid)-SSPHSY-NH₂ | SS |
| 124 | Pal-KVQKLAHQIYQFTD(Aib)DSAPVDPSSPHSY- | SS |
| | NH₂ | |
| 125 | Pal-KVQKLAHQIYQFTDK(Aib)SAPVDPSSPHSY- | AS/IS |
| | NH₂ | |
| 126 | Pal-KVQKLAHQIYQFTDKD(Aib)APVDPSSPHSY- | SS |
| | NH₂ | |
| 127 | Pal-KVQKLAHQIYQFTDKDS(Aib)PVDPSSPHSY- | LG |
| | NH₂ | |
| 128 | Pal-KVQKLAHQIYQFTDKDSA(Aib)VDPSSPHSY- | LG |
| | NH₂ | |
| 129 | Pal-KVQKLAHQIYQFTDKDSAP(Aib)DPSSPHSY- | LG |
| | NH₂ | |
| 130 | Pal-KVQKLAHQIYQFTDKDSAPV(Aib)PSSPHSY- | LG |
| | NH₂ | |
| 131 | Pal-KVQKLVAPRSKISPQGY-NH₂ | LG |
| 132 | Pal-VGSKAF-NH2 | AS/IS |
| 133 | Pal-VGSNTY-NH2 | AS/IS |
| 134 | Ace-NFVPTNVGPFAF-NH₂ | AS/IS |
| 135 | Pal-KVQKLNFVPTNVGSKAF-NH₂ | AS/IS |
| 136 | Pal-KVQKL(D-Asn)FVPTNVGSKAF-NH2 | AS/IS |
| 137 | Pal-KVQKLNFVPTNVGS-Lys(Met2)-AF-NH2 | AS/IS |
| 138 | Pal-KVQKLNFVPTNVGSK(D-Ala)F-NH2 | AS/IS |
| 139 | Pal-KVQKLNFVPTNVGSKAY-NH2 | SS |
| 140 | Ace-KVQKLNFVPTNVGSKAF-NH2 | LG |
| 141 | Ace-FKFEFKFE-NH₂ | SS |
| 142 | Ace-RADARADARADARADA-NH₂ | SS |

Abbreviations used in the Table include SS for semisolid gel, LG for liquid gel, and AS/IS for aqueous solution or insoluble. Polypeptides that remain as clear aquous solution or as insoluble particles appear to lack the ability to form a gel nanostructure.

Based on the finding that short sequence motifs in adrenomedullin family peptides form gel at low concentrations, we tested the hypothesis that the inclusion of such gel-forming-enhancing motifs could promote the gel-forming capability of other polypeptides. We tested the the ability to form semisolid or liquid gel by a series of chimeric analogs. These analogs included the gel-forming-enhancing motifs found in SEQ ID NOS: 109, 119 and 120. The gel-forming-enhancing motifs in these polypeptides range from 3 to 17 amino acids in length. These motifs included those that are appended to the N-terminus, C-terminus, or present within the sequence as amino acid substitutions, or appended to a side chain of an amino acid. The motifs were either linked directly or via a miniPEG linker to the functional polypeptides.

As shown in Table 1, kisspeptin, a kappa opioid receptor agonist, Sermorelin, compstatin, a bombesin receptor antagonist, gamma-MSH, and thymosin alpha1 peptides (SEQ ID NOS 6, 7, 8, 11, 13, 61, and 64) form self-assembling gels by themselves at 11% w/w or lower concentrations, and the conjugation or substitution with a gel-forming-enhancing motif in these polypeptides (Table 3, SEQ ID NOS 208, 214, 235, 244, 255, and 260) does not drastically alter the gel-forming capability of these polypeptides at the 11% w/w concentration. While there is no significant improvement in the gel-forming capability of these modified peptides, they represent superior analogs and are expected to have improved pharmacokinetic and pharmacodynamic characteristics when compared to the wild-type counterparts because these modified gel-forming peptides would have a longer circulating half-life due to the presence of an albumin-binding acyl group together with the gel-forming capability. Likewise, we showed that acylated analogs of ADM2-52 and thymosin beta 4 form gels at 11% w/w, and these acylated analogs represent superior analogs in having an albumin-binding acyl group and the ability to form gels by themselves.

On the other hand, we demonstrated dramatical changes in gel-forming capability for analogs of GnRH, GnRH antagonist, vasopressin, bombesin, apelin, bombesin receptor antagonist, deltorphin, enkephalin, substance P, calcitonin, Pramlintide (amylin analog), exenatide 4, GLP-1, Teduglutide (GLP-2 analog), afamelanotide (melanotan I), melanotan II, ACTH1-24, setmelanotide, urocortin 2, parathyroid hormone, PYY3-36, VIP, HOE140 (a bradykinin receptor 2 antagonist), bradykinin receptor 1 antagonist, sermorelin, atrial natriuretic peptide (ANP), adrenomedullin, thymosin α1a kallikrein inhibitor, antimicrobial temporin A, immunomodulating Glatiramer peptide (or Copaxone), a matrix modifying peptide 1, a matrix modifying peptide 4, a matrix modifying peptide 7, a matrix modifying peptide 8, and an acetyl hexapeptide-3 matrix modifying peptide (SEQ ID NOS:201-204, 209-213, 215, 217-228, 230, 232-234, 237-238, 242, 245-250, 253-254, 257, 261, 264, 266-270, 273-274). The inclusion of a gel-forming-enhancing motif in these peptides allows the formation of semisolid gel at a concentration (i.e., at 30%, 20%, 11%, or 6% w/w) that does not allow for semisolid gel formation by the wild-type counterpart. The phase transition can be observed within 20 min after solubilization. At the selected concentration, the wild-type polypeptides only form liquid gel, aqueous solution, or stay as insoluble precipitates.

In addition, we found that the addition of select gel-forming-enhancing motifs to oxytocin, apelin, neurotensin, saralasin, PYY3-36, melanotin I, gamma-MSH, urocortin 2, or a TAT cell penetrating-enhancing peptide partially improve the gel-forming capability of these polypeptides (SEQ ID NOS:205-207, 216, 229, 241, 256, 260, 262, and 265). In these cases, the modified polypeptides become liquid gels at the selected concentration while the wild-type counterparts (SEQ ID NOS:5, 18, 19, 24, 29, 30, 31, 36, and 64) only form aqueous solution or are insoluble. However, these modified polypeptides were unable to form semisolid gels at the selected concentrations tested. These data thus demonstrates that these analogs of GnRH, GnRH antagonist, vasopressin, bombesin, bombesin receptor antagonist, deltorphin, enkephalin, substance P, calcitonin, Pramlintide (amylin analog), exenatide 4, GLP-1, Teduglutide (GLP-2 analog), afamelanotide (melanotan I), melanotan II, gamma-MSH, ACTH1-24, setmelanotide, urocortin 2, parathyroid hormone, VIP, a bradykinin receptor antagonist, HOE140, sermorelin, atrial natriuretic peptide (ANP), thymosin alpha-1, thymosin beta 4, adrenomedullin, a kallikrein inhibitor, antimicrobial temporin A, compstatin, Glatiramer peptide (or Copaxone), a matrix modifying peptide 1, a matrix modifying peptide 4, a matrix modifying peptide 7, a matrix modifying peptide 8, and an acetyl hexapeptide-3 matrix modifying peptide as well as those of oxytocin, apelin, neurotensin saralasin, PYY3-36, a TAT cell penetrating-enhancing peptide, Pralmorelin, or urocortin 3 represent self-assembly gel-forming polypeptides and can be used for sustained release of a therapeutic agent that target their receptors, enzyme substrates, cellular compartment, or biological mediators in humans.

Abbreviations used in the Table include SS for semisolid gel, LG for liquid gel, and AS/IS for aqueous solution or insoluble.

TABLE 3
The identification of gel-forming enhancing motif-containing chimeric polypeptides that form
gel by itself in aqueous solution.

| SEQ | | | | | | |
|---|---|---|---|---|---|---|
| ID | | | | | | |
| NO | Sequence | 30% | 20% | 11% | 6% | 3% |

Chimeric or mutated polypeptides

| | | | | | | | |
|---|---|---|---|---|---|---|---|
| 201 | Pal-KVQKLSAPVDPSSPHSY-miniPEG-EHWSY-(D-2Nal)- | | | SS | SS | SS | GnRH |
| | LRPG-NH2 | | | | | | |
| 202 | Pal-KVQKLSAPVDPSSPHSY-miniPEG-(D-Nal)-(D-Cpa)- | | | SS | SS | AS/IS | GnRH antagonist |
| | (D-Pal)-SY-(D-Cit)-LRP-(D-Ala)-NH2 | | | | | | |
| 203 | Pal-HSY-(D-Nal)-(D-Cpa)-(D-Pal)-SY-(D-Cit)- | | | SS | SS | LG | GnRH antagonist |
| | LRP-(D-Ala)-NH2 | | | | | | |
| 204 | Pal-KVQKLSAPVDPSSPHSY-miniPEG-CYFQNCPRG-NH2 | | | SS | SS | | Vasopressin |
| 205 | Pal-KVQKLSAPVDPSSPHSY-miniPEG-CYIQNCPLG-NH2 | | | SS | LG | | Oxytocin |
| 206 | (G1p)RPRLSHKGP(Nle)PF-miniPEG- | | LG | LG | | | Apelin |
| | K(Pal)VQKLSAPVDPSSPHSY-NH2 | | | | | | |
| 207 | Pal-KVQKLSAPVDPSSPHSY-miniPEG-ELYENKPRRPYIL | | LG | LG | | | Neurotensin |
| 208 | Pal-KVQKLSAPVDPSSPHSY-miniPEG-YNWNSFGLRF-NH2 | | SS | LG | | | Kisspeptin |
| 209 | Pal-KVQKLSAPVDPSSPHSY-miniPEG- | | | SS | SS | SS | Bombesin |
| | (D-Tyr)QWAV(β-Ala)HF(Nle)-NH2 | | | | | | |
| 210 | Pal-KVQKLSAPVDPSSPHSY-miniPEG-Y-(D-Met)-FHLMD-NH2 | | | SS | SS | | Deltorphin |
| 211 | Pal-HSY-(D-Met)-FHLMD-NH2 | | | SS | LG | | Deltorphin |
| 212 | Pal-KVQKLSAPVDPSSPHSY-miniPEG-Y-(D-Ala)-GF(D-Leu)- | | | | | SS | Enkephalin |
| | NH2 | | | | | | |
| 213 | Pal-HSY-(D-Ala)-GF(D-Leu)-NH2 | | | | | SS | Enkephalin |
| 214 | Pal-SSPHSY-miniPEG-(D-Phe)-(D-Phe)-Trp-(D-Arg) | | | SS | SS | LG | Kappa opioid |
| | | | | | | | receptor |
| | | | | | | | agonist |
| 215 | Pal-KVQKLSAPVDPSSPHSY-miniPEG-RPKPQQFFGLM-NH2 | | | SS | AS/IS | | Substance P |
| 216 | Pal-KVQKLSAPVDPSSPHSY-miniPEG-Sar-RVYVHPA-NH2 | | LG | LG | | | Saralasin |
| 217 | Pal-KVQKLSAPVDPSSPHSY-miniPEG- | | | SS | LG | | Calcitonin |
| | CSNLSTCVLGKLSQELHKLQTYPRTNTGSGTP-NH2 | | | | | | |
| 218 | Pal-SSPHSY-miniPEG- | | | SS | SS | | Pramlintide |
| | KCNTATCATQRLANFLVHSSNNFGPILPPTNVGSNTY-NH2 | | | | | | |
| 219 | Pal-KVQKLSAPVDPSSPHSY-miniPEG- | | | SS | LG | | Exenatide 4 |
| | HGEGTFTSDLSKQMEEEAVRLFIEWLKNGGPSSGAPPPS-NH2 | | | | | | |
| 220 | Pal-KVQKLSAPVDPSSPHSY-miniPEG- | | | SS | LG | | GLP-1 |
| | HAEGTFTSDVSSYLEGQAAKEFIAWLVKGR-NH2 | | | | | | |
| 221 | Pal-HSYGTFTSDVSSYLEGQAAKEFIAWLVKGR-NH2 | | | | SS | LG | GLP-1 |
| 222 | Pal-SSPHSY-miniPEG- | | | SS | LG | | Teduglutide |
| | HGDGSFSDEMNTILDNLAARDFINWLIQTKITD-OH | | | | | | |
| 223 | Pal-SSPHSY-miniPEG -SYS-Nle-EH-(D-Phe)-RWGKPV-NH2 | | SS | LG | | | Afamelanotide |
| | | | | | | | (melanotan I) |
| 224 | Pal-HSYS-Nle-EH-(D-Phe)-RWGKPV-NH2 | | | SS | | | Afamelanotide |
| | | | | | | | (melanotan I) |
| 225 | Pal-SSPHSY-miniPEG-SYSMEHFRWGKPVGKKRRPVKVYP-OH | | SS | AS/IS | | | ACTH1-24 |
| 226 | Pal-HSYSMEHFRWGKPVGKKRRPVKVYP | | | SS | | | ACTH1-24 |
| 227 | Pal-SSPHSY-miniPEG-RC-(D-Ala)-H-(D-Phe)-RWC-NH2. | SS | | AS/IS | | | Setmelanotide |
| 228 | Pal-HSYRC-(D-Ala)-H-(D-Phe)-RWC-NH2 | | | SS | SS | SS | Setmelanotide |
| 229 | Pal-SSPHSY-miniPEG- | | LG | AS/IS | | | PYY3-36 |
| | AKPEAPGEDASPEELSRYYASLRHYLNLVTRQRY-NH2 | | | | | | |
| 230 | Pal-SSPHSY-miniPEG- | | SS | AS/IS | | | Urocortin 2 |
| | IVLSLDVPIGLLQILLEQARARAAREQATTNARILARVGHC-NH2 | | | | | | |
| 231 | Pal-KSYSLDVPTNIMNLLFNIAKAKNLRAQAAANAHLMAQI-NH2 | | | SS | SS | LG | Urocortin 3 |
| 232 | Pal-HSYSEIQLMHNLGKHLNSMERVEWLRKKLQDVHNF | | SS | LG | | | Parathyroid |
| | | | | | | | hormone |
| 233 | Pal-SSPHSY-miniPEG-(D-Arg)-Arg-Pro-Hyp-Gly-Thi- | SS | | AS/IS | | | HOE140 (BKR2 |
| | Ser-(D-Tic)-Oic-Arg | | | | | | antagonist) |
| 234 | Pal-HSY-(D-Arg)-Arg-Pro-Hyp-Gly-Thi-Ser-(D-Tic)- | | SS | AS/IS | AS/IS | | HOE140 (BKR2 |
| | Oic-Arg-NH2 | | | | | | antagonist) |
| 235 | Pal-SSPHSY-miniPEG-(D-Ala)-(D-(β-naphthyl))-AAW- | | | SS | SS | SS | Pralmorelin |
| | (D-Phe)-Lys-NH2 | | | | | | |
| 236 | Pal-SSPHSY-miniPEG-YADAIFTNSYRKVLGQLSARKLLQDIMSR-NH2 | | | AS/IS | AS/IS | | Sermorelin |
| 237 | Pal-HSYADAIFTNSYRKVLGQLSARKLLQDIMSR-NH2 | | SS | AS/IS | AS/IS | | Sermorelin |
| 238 | Pal-SSPHSY-miniPEG-SLRRSSCFGGRMDRIGAQSGLGCNSFRY-NH2 | | SS | SS | | | ANP |
| 239 | Pal-SSPHSY-miniPEG-SDAAVDTSSEITTKDLKEKKEVVEEAEN-NH2 | | | AS/IS | | | Thymosin alpha-1 |
| 240 | Pal-HSYAAVDTSSEITTKDLKEKKEVVEEAEN-NH2 | | | SS | AS/IS | | Thymosin alpha-1 |
| 241 | FITC-K(Pal)SSPHSY-miniPEG-GRKKRRQRRRPQ | | | LG | | | Cell-penetrating |
| | | | | | | | peptide (TAT) |
| 242 | Pal-SSPHSY-miniPEG-PFRSVQ-NH2 | | | SS | LG | | Kallikrein |
| | | | | | | | inhibitor |
| 243 | Pal-SSPHSY-miniPEG-FLPLIGRVLSGIL-NH2 | | | SS | AS/IS | | Temporin A |
| 244 | Pal-SSPHSY-miniPEG-ICVVQDWGHHRCT-NH2 | | | SS | AS/IS | | Compstatin |
| 245 | Pal-SSPHSY-miniPEG- | | SS | AS/IS | | | Glatiramer |
| | EAYKAAEKAYAAKEAAKEAAKAKAEKKAAYAKAKAAKYEKKAKKAAAEY | | | | | | peptide |
| | KKK | | | | | | (Copaxone) |
| 246 | Pal-SSPHSY-miniPEG-YIGSR-NH2 | | | SS | SS | | Matrix modifying |
| | | | | | | | peptide 1 |
| 247 | Pal-SSPHSY-miniPEG-RGD-NH2 | | | SS | | | Matrix modifying |
| | | | | | | | peptide 4 |
| 248 | Pal-SSPHSY-miniPEG-VGVAPG-NH2 | | SS | LG | | | Matrix modifying |
| | | | | | | | peptide 7 |
| 249 | Pal-SSPHSY-miniPEG-YRSRKYSSVVY-NH2 | | | SS | AS/IS | | Matrix modifying |
| | | | | | | | peptide 8 |
| 250 | Pal-HSYEEMQRR-NH2 | | | SS | AS/IS | | Acetyl |
| | | | | | | | hexapeptide-3 |
| 251 | B Chain: Pal-SSPHSY-FVNQHLCGSHLVEALYLVCGERGFFYTPKT | | | | | | Insulin |
| | A Chain: GIVEQCCTSICSLYQLENYCN | | | | | | |
| 252 | B Chain: Pal-SSPHSY-DSWMEEVIKLCGRELVRAQIAICGMSTWS | | | | | | Relaxin 2 |
| | A Chain: pGlu-LYSALANKCCHVGCTKRSLARFC | | | | | | |
| 253 | Pal-HSYFFGLM-NH2 | | | SS | AS/IS | | Substance P |
| 254 | Pal-HSYRPKPQQFF-Sar-L-Met-(02)-NH2 | | SS | LG | | | Substance P |
| 255 | Pal-HSY-(D-Phe)-QWAVGHL-(4-Cl)Phe-NH2 | | | SS | LG | AS/IS | Bombesin receptor |
| | | | | | | | antagonist |
| 256 | Pal-HSYS-Nle-EH-(D-Phe)-RWGKPVGKKRRPVKVYP-NH2 | | LG | LG | | | Afamelanotide |
| | | | | | | | (melanotan I) |
| 257 | Pal-HSY-Nle-cyclo[Asp-His-D-Phe-Arg-Trp-Lys]-NH2 | | SS | SS | LG | | Melanotan II |
| 258 | Pal-HSYAVSEHQLLHDKGKSIQDLRRRFFLHHLIAEIHTA-NH2 | | | | | | PTHrP 1-34 |
| 259 | Pal-HSY-Nle-cyclo[Asp-His-D-Phe-Arg-Trp-Lys]- | | | | | | Melanotan II |
| | PVGKKRRPVKVYP-NH2 | | | | | | |
| 260 | Pal-HSYVMGHFRWDRF-NH2 | | | LG | LG | | Gamma-MSH |
| 261 | Pal-HSYKRPPGFS-(DβNal)-Ile-NH2 | | | SS | LG | AS/IS | Bradykinin |
| | | | | | | | receptor |
| | | | | | | | antagonist |
| 262 | (G1p)RPRLSHKGP(Nle)PF-K(Pal)HSY-NH2 | | LG | LG | LG | AS/IS | Apelin |
| 263 | Pal-HSDKPDMAEIEKFDKSKLKKTETQEKNPLPSKETIEQEKQAGES-NH2 | | LG | LG | | | Thymosin beta 4 |
| 264 | Pal-HSYAKPEAPGEDASPEELSRYYASLRHYLNLVTRQRY-NH2 | | SS | SS | AS/IS | | PYY3-36 |
| 265 | Pal-HSYSLDVPIGLLQILLEQARARAAREQATTNARILARVGHC-NH2 | | LG | AS/IS | | | Urocortin 2 |
| 266 | HSDAVFTDNYTRLRKQ-Nle-AVKKYLNSILN-(Pal-K)-SY-NH2 | | | SS | | | VIP |
| 267 | Pal-HSDAAVDTSSEITTKDLKEKKEVVEEAEN-NH2 | | | SS | SS | | Thymosin alpha-1 |
| 268 | Pal-SDAAVDTSSEITTKDLKEKKEVVEEAEN-NH2 | | | SS | LG | | Thymosin alpha-1 |
| 269 | HAEGTFTSDVSSYLEGQAAK(YSHPSS-Pal)EFIAWLVRGRG | | SS | SS | LG | | GLP-1 |
| 270 | HAEGTFTSDVSSYLEGQAAREFIAWLVK(YSHPSS-Pal)GRG | | SS | SS | LG | | GLP-1 |
| 271 | HGEGTFTSDVSSYLEGQAAK(YSH-Pal)EFIAWLVRGRG | | | | | | GLP-1 |
| 272 | HGEGTFTSDLSRQMEEEAVK(YSH-Pal)LFIEWLRNGGPSSGAPPPS | | | | | | Exenatide 4 |
| 273 | Pal-HSYH-(D-Phe)-RWGKPV-NH2 | | SS | SS | LG | | Afamelanotide |
| | | | | | | | (melanotan I) |
| 274 | Pal- | | SS | SS | SS | | adrenomedullin |
| | RQSMNNFQGLRSFGCRFGTCTVQKLAHQIYQFTDKDKDNVAPRSKISPQGY- | | | | | | 1-52 |
| | NH2 | | | | | | |
| 274 | Pal- | | SS | SS | SS | | adrenomedullin |
| | RQSMNNFQGLRSFGCRFGTCTVQKLAHQIYQFTDKDKDNVAPRSKISPQGY- | | | | | | 14-52 analog |
| | NH2 | | | | | | (ADE43) |
| 275 | Pal- | | | | | | adrenomedullin |
| | MNNFQGLRSFGCRFGTCTVQKLAHQIYQFTDKDKDNVAPRSKISPQGY-NH2 | | | | | | 14-52 analog |
| | | | | | | | (ADE43) |
| 284 | FKFEFKFE-miniPEG-RPKPQQFFGLM-NH2 | | | AS/IS | AS/IS | | Substance P |
| 285 | QQRFEWEFEQQ-miniPEG-RPKPQQFFGLM-NH2 | | | AS/IS | AS/IS | | Substance P |
| 286 | VKVKVKVKV(D-Pro)PTKVKVKVKV-miniPEG-RPKPQQFFGLM-NH2 | | | AS/IS | AS/IS | | Substance P |

Wild-type Counterparts

| | | | | | | | |
|---|---|---|---|---|---|---|---|
| 15 | (G1p)HWSY-(D-2Nal)-LRPG-NH2 | | | LG | AS/IS | | GnRH |
| 27 | (D-Nal)-(D-Cpa)-(D-Pal)-SY-(D-Cit)-LRP-(D-Ala)-NH2 | | | AS/IS | | | GnRH antagonist |
| 27 | (D-Nal)-(D-Cpa)-(D-Pal)-SY-(D-Cit)-LRP-(D-Ala)-NH2 | | | AS/IS | | | GnRh antagonist |
| 17 | CYFQNCPRG-NH2 | | | AS/IS | | | Vasopressin |
| 5 | CYIQNCPLG-NH2 | | | SS | AS/IS | | Oxytocin |
| 18 | (G1p)RPRLSHKGP(Nle)PF-NH2 | | AS/IS | AS/IS | AS/IS | | Apelin |
| 19 | (G1p)LYENKPRRPYIL | | AS/IS | AS/IS | | | Neurotensin |
| 6 | YNWNSFGLRF-NH2 | | | SS | SS | | Kisspeptin |
| 20 | (D-Tyr)QWAV(β-Ala)HF(Nle)-NH2 | | | AS/IS | AS/IS | | Bombesin |
| 21 | Y-(D-Met)-FHLMD-NH2 | | | AS/IS | AS/IS | | Deltorphin |
| 21 | Y-(D-Met)-FHLMD-NH2 | | | AS/IS | AS/IS | | Deltorphin |
| 22 | Y-(D-Ala)-GF(D-Leu)-NH2 | | | AS/IS | AS/IS | | Enkephalin |
| 22 | Y-(D-Ala)-GF(D-Leu)-NH2 | | | AS/IS | AS/IS | | Enkephalin |
| 7 | (D-Phe)-(D-Phe)-Trp-(D-Arg) | | | SS | SS | SS | Kappa opioid |
| | | | | | | | receptor agonist |
| 23 | RPKPQQFFGLM-NH2 | | LG | AS/IS | | | Substance P |
| 24 | Sar-RVYVHPA-NH2 | | AS/IS | | | | Saralasin |
| 28 | CSNLSTCVLGKLSQELHKLQTYPRTNTGSGTP-NH2 | | AS/IS | | | | Calcitonin |
| 4 | KCNTATCATQRLANFLVHSSNNFGPILPPTNVGSNTY-NH2 | | SS | AS/IS | | | Pramlintide |
| 26 | HGEGTFTSDLSKQMEEEAVRLFIEWLKNGGPSSGAPPPS-NH2 | | | AS/IS | | | Exenatide 4 |
| 14 | HAEGTFTSDVSSYLEGQAAKEFIAWLVKGR-NH2 | | | LG | AS/IS | | GLP-1 |
| 14 | HAEGTFTSDVSSYLEGQAAKEFIAWLVKGR-NH2 | | | LG | AS/IS | | GLP-1 |
| 12 | HGDGSFSDEMNTILDNLAARDFINWLIQTKITD-OH | | | LG | AS/IS | | Tedglutide |
| 29 | SYS-Nle-EH-(D-Phe)-RWGKPV-NH2 | | AS/IS | AS/IS | AS/IS | | Afamelanotide |
| | | | | | | | (melanotan I) |
| 29 | SYS-Nle-EH-(D-Phe)-RWGKPV-NH2 | | AS/IS | AS/IS | AS/IS | | Afamelanotide |
| | | | | | | | (melanotan I) |
| 33 | SYSMEHFRWGKPVGKKRRPVKVYP-OH | | AS/IS | AS/IS | | | ACTH1-24 |
| 33 | SYSMEHFRWGKPVGKKRRPVKVYP-OH | | AS/IS | AS/IS | | | ACTH1-24 |
| 34 | RC-(D-Ala)-H-(D-Phe)-RWC-NH2. | AS/IS | | AS/IS | | | Setmelanotide |
| 34 | RC-(D-Ala)-H-(D-Phe)-RWC-NH2. | AS/IS | | AS/IS | | | Setmelanotide |
| 30 | AKPEAPGEDASPEELSRYYASLRHYLNLVTRQRY-NH2 | | AS/IS | AS/IS | | | PYY3-36 |
| 31 | IVLSLDVPIGLLQILLEQARARAAREQATTNARILARVGHC-NH2 | | AS/IS | AS/IS | | | Urocortin 2 |
| 10 | FTLSLDVPTNIMNLLFNIAKAKNLRAQAAANAHLMAQI-NH2 | | SS | SS | LG | | Urocortin 3 |
| 25 | SVSEIQLMHNLGKHLNSMERVEWLRKKLQDVHNF | | | AS/IS | AS/IS | | Parathyroid |
| | | | | | | | hormone |
| 32 | (D-Arg)-Arg-Pro-Hyp-Gly-Thi-Ser-(D-Tic)-Oic-Arg | AS/IS | AS/IS | AS/IS | | | HOE140 (BKR2 |
| | | | | | | | antagonist) |
| 32 | (D-Arg)-Arg-Pro-Hyp-Gly-Thi-Ser-(D-Tic)-Oic-Arg | AS/IS | AS/IS | AS/IS | | | HOE140 (BKR2 |
| | | | | | | | antagonist) |
| 8 | (D-Ala)-(D-(β-naphthyl))-AAW-(D-Phe)-Lys-NH2 | | | SS | SS | LG | Pralmorelin |
| 35 | YADAIFTNSYRKVLGQLSARKLLQDIMSR-NH2 | | AS/IS | AS/IS | | | Sermorelin |
| 35 | YADAIFTNSYRKVLGQLSARKLLQDIMSR-NH2 | | AS/IS | AS/IS | | | Sermorelin |
| 41 | SLRRSSCFGGRMDRIGAQSGLGCNSFRY-NH2 | | AS/IS | AS/IS | | | ANP |
| 13 | SDAAVDTSSEITTKDLKEKKEVVEEAEN-NH2 | | SS | LG | | | Thymosin alpha-1 |
| 13 | SDAAVDTSSEITTKDLKEKKEVVEEAEN-NH2 | | SS | LG | | | Thymosin alpha-1 |
| 36 | GRKKRRQRRRPQ | | AS/IS | AS/IS | | | Cell-penetrating |
| | | | | | | | peptide (TAT) |
| 37 | PFRSVQ-NH2 | | | AS/IS | | | Kallikrein |
| | | | | | | | inhibitor |
| 68 | FLPLIGRVLSGIL-NH2 | | | | | | Temporin A |
| 11 | ICVVQDWGHHRCT-NH2 | | | SS | | | Compstatin |
| 42 | EAYKAAEKAYAAKEAAKEAAKAKAEKKAAYAKAKAAKYEKKAKKAAAEY | | AS/IS | AS/IS | | | Glatiramer peptide |
| | KKK | | | | | | (Copaxone) |
| 39 | YIGSR-NH2 | | | AS/IS | | | Matrix modifying |
| | | | | | | | peptide 1 |
| 44 | RGD-NH2 | | | AS/IS | | | Matrix modifying |
| | | | | | | | peptide 4 |
| 45 | VGVAPG-NH2 | | | AS/IS | AS/IS | | Matrix modifying |
| | | | | | | | peptide 7 |
| 46 | YRSRKYSSVVY-NH2 | | | AS/IS | | | Matrix modifying |
| | | | | | | | peptide 8 |
| 47 | Ac-EEMQRR-NH2 | | | AS/IS | | | Acetyl |
| | | | | | | | hexapeptide-3 |
| 59 | B Chain: FVNQHLCGSHLVEALYLVCGERGFFYTPKT | | | | | | Insulin |
| 287 | A Chain: GIVEQCCTSICSLYQLENYCN | | | | | | |
| 60 | B Chain: DSWMEEVIKLCGRELVRAQIAICGMSTWS | | | | | | Relaxin 2 |
| 288 | A Chain: pGlu-LYSALANKCCHVGCTKRSLARFC | | | | | | |
| 23 | RPKPQQFFGLM-NH2 | | LG | AS/IS | | | Substance P |
| 69 | RPKPQQFF-Sar-L-Met-(O2)-NH2 | | AS/IS | AS/IS | AS/IS | | Substance P |
| 61 | (D-Phe)-QWAVGHL-(4-CI)Phe-NH2 | | | SS | SS | | Bombesin receptor |
| | | | | | | | antagonist |
| 29 | SYS-Nle-EH-(D-Phe)-RWGKPV-NH2 | | AS/IS | AS/IS | AS/IS | | Afamelanotide |
| | | | | | | | (melanotan I) |
| 62 | Ac-Nle-cyclo[Asp-His-D-Phe-Arg-Trp-Lys]-NH2 | | AS/IS | AS/IS | | | Melanotan II |
| 63 | AVSEHQLLHDKGKSIQDLRRRFFLHHLIAEIHTA-NH2 | | | | | | PTHrP 1-34 |
| 62 | Ac-Nle-cyclo[Asp-His-D-Phe-Arg-Trp-Lys]-NH2 | | AS/IS | AS/IS | | | Melanotan II |
| 64 | YVMGHFRWDRF-NH2 | | | LG | AS/IS | | Gamma-MSH |
| 65 | KRPPGFS-(Dβal)-Ile-NH2 | | | AS/IS | AS/IS | | Bradykinin |
| | | | | | | | receptor |
| | | | | | | | antagonist |
| 18 | (G1p)RPRLSHKGP(Nle)PF-NH2 | | AS/IS | AS/IS | AS/IS | | Apelin |
| 66 | SDKPDMAEIEKFDKSKLKKTETQEKNPLPSKETIEQEKQAGES | | | | | | Thymosin beta 4 |
| 30 | AKPEAPGEDASPEELSRYYASLRHYLNLVTRQRY-NH2 | | AS/IS | AS/IS | | | PYY3-36 |
| 31 | IVLSLDVPIGLLQILLEQARARAAREQATTNARILARVGHC-NH2 | | AS/IS | AS/IS | | | Urocortin 2 |
| 67 | HSDAVFTDNYTRLRKQMAVKKYLNSILN | | AS/IS | AS/IS | | | VIP |
| 13 | SDAAVDTSSEITTKDLKEKKEVVEEAEN-NH2 | | SS | LG | | | Thymosin alpha-1 |
| 13 | SDAAVDTSSEITTKDLKEKKEVVEEAEN-NH2 | | SS | LG | | | Thymosin alpha-1 |
| 14 | HAEGTFTSDVSSYLEGQAAKEFIAWLVKGR-NH2 | | | LG | AS/IS | | GLP-1 |
| 14 | HAEGTFTSDVSSYLEGQAAKEFIAWLVKGR-NH2 | | | LG | AS/IS | | GLP-1 |
| 14 | HAEGTFTSDVSSYLEGQAAKEFIAWLVKGR-NH2 | | | LG | AS/IS | | GLP-1 |
| 26 | HGEGTFTSDLSKQMEEEAVRLFIEWLKNGGPSSGAPPPS-NH2 | | | AS/IS | | | Exematide |
| 29 | SYS-Nle-EH-(D-Phe)-RWGKPV-NH2 | | AS/IS | AS/IS | AS/IS | | Afamelanotide |
| | | | | | | | (melanotan I) |
| 2 | YRQSMNNFQGLRSFGCRFGTCTVQKLAHQIYQFTDKDKDNVAPRSKISPQGY- | | | SS | AS/IS | AS/IS | Adrenomedullin |
| | NH2 | | | | | | 1-52 |
| 1 | Ace-K(PAL)FGCRFGTCTVQKLAHQIYQFTDKDKDNVAPRSKISPQGY-NH2 | | | LG | AS/IS | AS/IS | Adrenomedullin |
| | | | | | | | 14-52 analogs |
| | | | | | | | (ADE43) |
| 1 | Ace-K(PAL)FGCRFGTCTVQKLAHQIYQFTDKDKDNVAPRSKISPQGY-NH2 | | | LG | AS/IS | AS/IS | Adrenomedullin |
| | | | | | | | 14-52 analogs |
| | | | | | | | (ADE43) |

Based on the visual inspection, tube tapping assay and the tilted tube assay, we showed that a GnRH analog (SEQ ID NO:15; Table 3) forms liquid gel in distilled water at 11% w/w. By contrast, the gel-forming-enhancing motif-conjugated GnRH analog (SEQ ID NO:201) forms semisolid gel at 11, 6, or 3% w/w. Likewise, while a GnRH antagonist (SEQ ID NO: 27) does not form gel, the gel-forming-enhancing motif-conjugated GnRH antagonists (SEQ ID NOS:202 and 203) form semisolid gel at 11 and 6% w/w. The wild-type vasopressin (SEQ ID NO:17) does not form gel at 11%, whereas the gel-forming-enhancing motif-conjugated vasopressin (SEQ ID NO:204) forms semisolid gel at 11 and 6% w/w. The wild type oxytocin (SEQ ID NO:5) is able to forms semisolid gel at 11%, but does not form liquid or semisolid gel at 6% w/w. On the other hand, the gel-forming-enhancing motif-conjugated oxytocin analog (SEQ ID NO:205) forms semisolid gel at 11% and liquid gel at 6% w/w.

The wild-type apelin (SEQ ID NO:18) does not form gel at 20, 11, or 6% w/w. On the other hand, the gel-forming-enhancing motif conjugated apelin analog (SEQ ID NOS:206 and 262) forms liquid gel at 20 or 11% w/w. Likewise, the wild-type neurotensin (SEQ ID NO:19) does not form gel at 20 or 11% w/w, whereas the gel-forming-enhancing motif-conjugated neurotensin analog (SEQ ID NO:207) forms liquid gel at 20 and 11% w/w. The wild type bombesin (SEQ ID NO:20) does not form gel at 11 or 6% w/w. On the other hand, the conjugation of a gel-forming-enhancing motif to bombesin or a bombesin receptor antagonist leads to analogs (SEQ ID NOS:209 and 255) that form semisolid or liquid gel at 11, 6 or 3% w/w. Unlike apelin, neurotensin and bombesin, the kisspeptin peptide (SEQ ID NO:6) forms semisolid gel at 11 or 6% w/w, and the gel-forming-enhancing motif-conjugated kisspeptin analog (SEQ ID NO:208) forms gel at the same concentrations.

The ligands for opioid receptors, including deltrophin and endephalin (SEQ ID NOS:21 and 22), do not form gels at 11 or 6% w/w. On the other hand, the gel-forming-enhancing motif conjugated deltorphin analogs (SEQ ID NOS:210 and 211) are able to form semisolid gel at 11% and semisolid or liquid gel at 6% w/w. The gel-forming-enhancing motif conjugated enkephalin analogs (SEQ ID NOS:212 and 213) can form semisolid gels at even 3% w/w. Nonetheless, a kappa opioid receptor agonist (SEQ ID NO:7) can form semisolid gel at 11, 6 or 3% w/w. The corresponding gel-forming-enhancing motif conjugated analog (SEQ ID NO:214) retains the ability to form semisolid gel at 11 and 6% w/w, and to form liquid gel at 3% w/w.

The wild-type substance P analogs (SEQ ID NOS:23 and 69) only forms liquid gel at 20% w/w or not at all. When the substance P was conjugated with a gel-forming-enhancing motif, the conjugated analog (SEQ ID NOS:215, 253, and 254) become able to form semisolid gel at 11% or 20% w/w. The angiotensin receptor antagonist saralasin (SEQ ID NO:24) does not form gel at 11% w/w. When the gel-forming-enhancing motif is conjugated to saralasin causes the resulting analog (SEQ ID NO:216) to obtain the capability to form liquid gel at 20 or 11% w/w.

The wild-type calcitonin (SEQ ID NO:28) does not form gel at 11% w/w. On the other hand, the gel-forming-enhancing motif conjugated calcitonin (SEQ ID NO:217) is able to form semisolid gel at 11% and liquid gel at 6% w/w. The amylin peptide analog, Pramlinitide (SEQ ID NO:4), does not form gel at 6% but form semisolid gel at 11%. When the Pramlinitide is conjugated with a gel-forming-enhancing motif, the resulting analog (SEQ ID NO:218) is capable of forming semisolid gel at 11 or 6% w/w. Unlike exenatide 4 (SEQ ID NO:26), which does not form gel at 11% w/w, the gel-forming-enhancing motif conjugated exenatide 4 analog (SEQ ID NO:219) forms semisolid gel at 11% and liquid gel at 6% w/w. The wild-type GLP-1 (SEQ ID NO:14) only forms liquid gel at 11%. On the other hand, the gel-forming-enhancing motif conjugated GLP-1 analogs (SEQ ID NOS:220, 269, and 270) and gel-forming-enhancing motif-substituted analog (SEQ ID NO:221) form semisolid gels at 20 or 11% w/w. The GLP-2 analog, Tedglutide (SEQ ID NO:12) forms liquid gel at 11%. On the other hand, the gel-forming-enhancing motif conjugated GLP-2 analog (SEQ ID NO:222) forms semisolid gel at 11% and liquid gel at 6% w/w.

The melanotan I analog afamelanotide and melanotan II analog (SEQ ID NOS:29 and 62) do not form gel at 20 or 11% w/w. By contrast, the gel-forming-enhancing motif conjugated afamelanotide analogs (SEQ ID NOS:223, 224, 256, 257, and 273) are able to form semisolid or liquid gel at 20, 11, or 6% w/w. The ACTH1-24 peptide (SEQ ID NO:33) does not form gel at 20 or 11% w/w. By contrast, the gel-forming-enhancing motif conjugated ACTH1-24 analogs (SEQ ID NOS:225 and 226) can form semisolid gel at 20 or 11% w/w. The setmelanotide peptide (SEQ ID NO:34), an agonist of the melanocortin 4 receptor (MC4R), does not form gel at 30, 20, or 11% w/w. When the setmelanotide peptide is fused with a Pal-SSPHSY gel-forming-enhancing motif, the resulting analog (SEQ ID NO:227) is able to form gel at 30% w/w. On the other hand, the setmelanotide analog (SEQ ID NO:228) with a Pal-HSY gel-forming-enhancing motif forms semisolid gel at 11, 6, or 3% w/w. In addition, we found that the gamma-MSH (SEQ ID NO: 64) forms liquid gel at 11% and stay soluble at 6% w/w. The gel-forming-enhancing motif-conjugated gamma MSH analog (SEQ ID NO:260) was able to form liquid gel at 6% w/w.

The peptide YY receptor agonist PYY3-36 (SEQ ID NO:30) does not form gel at 20 or 11% w/w. When the peptide is conjugated with a gel-forming-enhancing motif, the resulting analog (SEQ ID NOS:229 and 264) gains the ability to form semisolid or liquid gel at 20% or 11% w/w. The corticotropin receptor 2 (CRHR2) agonist urocortin 2 (UCN2) peptide (SEQ ID NO:31) does not form gel at 20 or 11% w/w. The gel-forming-enhancing motif conjugated urocortin 2 analogs (SEQ ID NOS:230 and 265), by contrast, gains the ability to form semisolid or liquid gel at 20% w/w. Unlike UCN2, the urocortin 3 (UCN3) peptide (SEQ ID NO:10) forms semisolid gel at 11 and 6% and forms liquid gel at 3% w/w. The gel-forming-enhancing motif conjugated UCN3 analog (SEQ ID NO:231) retains the ability to form gel at these concentrations. On the other hand, the parathyroid hormone peptide (SEQ ID NO:25) does not form gel at 11 or 6% w/w. When the N-terminus of this peptide is substituted with a gel-forming-enhancing motif, the resulting analog (SEQ ID NO:232) gains the ability to form semisolid gel at 20% and liquid gel at 11% w/w. The bradykinin receptor ligand HOE140, an antagonist of BKR2 (SEQ ID NO:32), does not form gel at 30, 20 or 11% w/w. However, when the peptide is conjugated with a Pal-SSPHSY gel-forming-enhancing motif, the resulting analog (SEQ ID NO:233) gains the ability to form semisolid gel at 30% w/w. When the HOE140 is conjugated with a Pal-HSY gel-forming-enhancing motif, the resulting analog (SEQ ID NO:234) is able to form semisolid gel at 20% w/w. Another bradykinin receptor antagonist, SEQ ID NO 65 does not form a gel at 11% or 6% w/w, whereas, the modified analog SEQ ID NO 261 forms semisolid gel at 11% and liquid gel at 6% w/w.

The Pralmorelin peptide (SEQ ID NO:8) forms semisolid gel at 11 or 6% w/w and liquid gel at 3% w/w. Similar to the wild-type Pralmorelin peptide, the gel-forming-enhancing motif conjugated Pralmorelin analog (SEQ ID NO:235) forms semisolid gel at the same concentrations. Unlike Pralmorelin, the sermorelin peptide (SEQ ID NO:35) does not form gel at 20 or 11% w/w. The sermorelin analog gains the ability to form semisolid gel at 20% when it is fused with a short gel-forming-enhancing motif (SEQ ID NO:237) but not when it is fused with a long gel-forming-enhancing motif (SEQ ID NO:236).

The atrial natriuretic peptide (ANP)(SEQ ID NO:41) does not form gel at 20 or 11% w/w. By contrast, the gel-forming-enhancing motif conjugated ANP analog (SEQ ID NO:238) gains the ability to form semisolid gel at 20 and 11% w/w. The immunoregulator thymosin alpha 1 (SEQ ID NO:13) is capable of forming semisolid gel at 20% and liquid gel at 11% w/w. When the N-terminus of thymosin alpha 1 is substituted with a gel-forming-enhancing motif (Pal-HSY) or an even shorter gel-forming motif, it retains the ability to form semisolid gel at 11% w/w (SEQ ID NOS:240, 267 and 268). On the other hand, the analog (SEQ ID NO:239) with a long gel-forming-enhancing motif (Pal-SSPHSY) does not form gel at the same concentration. In addition, we showed that a thymosin beta 4 analog, SEQ ID NO 263, forms liquid gel at 11% and 20% w/w.

The vasoactive intestinal peptide (VIP) (SEQ ID NO:67) does not form gel at 20 or 11% w/w. By contrast, the gel-forming-enhancing motif conjugated VIP analog (SEQ ID NO:266) gains the ability to form semisolid gel at 11% w/w. Furthermore, we showed that the adrenomedullin analog SEQ ID NO 274 forms semisolid gel at 20, 11, or 6% w/w, which is superior to the wild-type adrenomedullin SEQ ID NO:2 or a shorter acylated analog (SEQ ID NO:1).

The cell-penetrating enhancing peptide (CPP) TAT by itself (SEQ ID NO:36) cannot form gel at 20 or 11% w/w. It gains the ability to form liquid gel at 11% w/w when it is fused with a gel-forming-enhancing motif (SEQ ID NO:241). Likewise, a kallikrein inhibitor analog (SEQ ID NO:37) does not form gel at 11% w/w. When the kallikrein inhibitor is fused with a gel-forming-enhancing motif, the resulting analog (SEQ ID NO:242) gains the ability to form semisolid gel at 11% and liquid gel at 6% w/w. The temporin A peptide, which is an antimicrobial peptide, is fused with a gel-forming-enhancing motif, the resulting analog (SEQ ID NO:243) forms semisolid gel at 11% w/w. On the other hand, the complement system regulator compstatin (SEQ ID NO:11) forms semisolid gel at 11% w/w, and its gel-forming-enhancing motif-containing analog (SEQ ID NO:244) forms semisolid gel at the same concentration. The immunoregulator Glatiramer peptide (or Copaxone)(SEQ ID NO:42) does not form gel at 20 or 11% w/w. When this peptide is conjugated with a gel-forming-enhancing motif, the resulting analog (SEQ ID NO:245) obtains the capability to form semisolid gel at 20% w/w.

Testing of a number of matrix modifying peptides (or matrikines; SEQ ID NOS:39 and 44-47) that can modulate skin matrix enzymes showed that these peptides lack the ability to form semisolid or liquid gel at 11% w/w. However, when they are fused with a gel-forming-enhancing motif, they gain the ability to form semisolid or liquid gel at 11% w/w. For example, the fusion peptide of SEQ ID NOS:246, 247, 249 and 250 all gain the ability to form semisolid gel at 11% w/w; whereas the fusion peptide of SEQ ID NO:248 obtains the ability to form liquid gel at 11% and semisolid gel at 20% w/w.

In a separate experiment, we examined the effects of conjugation of known gel-forming peptide motifs on the ability of substance P analogs to form semisolid or liquid gel. While the gel-forming-enhancing motif-containing substance P analog (SEQ ID NO:23) forms semisolid gel at 11% w/w, the addition of known gel-forming peptides, which contain repeated sequences (e.g., FKFEFKFE, QQRFEWEFEQQ, and VKVKVKVKV(D-Pro)PTKVKVKVKV), to the substance P did not lead to the generation of gel-forming analogs (SEQ ID NOS:284-286; Table 3).

To determine whether the modifications have effects on the bioactivity of gel-forming peptides, we studied the receptor-regulatory activities of select gel-forming peptides. Studies of the receptor-activation activities on MC1R showed that conjugated gel-forming Afamelanotide (melanotan I) and ACTH1-24 analogs (SEQ ID NOS:224 and 226) stimulate MC1R and exhibit EC50 at the subnanomolar and nanomolar ranges, respectively (Table 4). The positive control Melanotan II peptide has an EC50 of 0.56 nM. Analysis of the receptor-activation activities on MC4R showed that conjugated gel-forming setmelanotide analog (SEQ ID NO:228) stimulates MC4R with an EC50 of 0.15 nM. In this assay, the Melanotan II peptide has an EC50 of 2 nM, suggesting that select modified peptides also had a superior receptor-activation activity. Likewise, analysis of CRHR2 activities showed that gel-forming urocortin 2 and urocortin 3 analogs (SEQ ID NOS:230 and 231) potently stimulate CRHR2 and exhibit EC50 of 0.5 and 5.7 nM, respectively. The positive control sauvagine peptide exhibits an EC50 of 3.6 nM, suggesting that select modified urocortin peptide also had a superior receptor-activation activity. Studies of PTHR1 receptor activities showed that the gel-forming PTH analog (SEQ ID NO:232) has an EC50 of 14.1 nM on PTHR1 whereas the positive control PTH(1-34) peptide has an EC50 of 0.8 nM. On the other hand, the gel-forming kappa opioid receptor agonist (SEQ ID NO:214) stimulated OPRK1 with an EC50 of 272 nM, and the positive control dynorphin A peptide has an EC50 of 0.5 nM.

In addition, study of the activity of bradykinin receptor BDKRB2 using a Discoverx BDKRB2 arrestin assay showed that the conjugated bradykinin receptor antagonist, SEQ ID NO:234 inhibits bradykinin-stimulated BDKRB2 signaling and has an IC50 of 53.3 nM (Table 4). By contrast, the positive control antagonist, HOE140, has an IC50 of 131.8 nM, suggesting that the gel-forming antagonist is more potent than the wild-type peptide HOE140. Furthermore, analysis of the activity of GnRH receptor using a Discoverx GNRHR calcium flux assay showed that the gel-forming GnRHR antagonist analogs, SEQ ID NOS:202 and 203, inhibits LHRH-stimulated GnRHR signaling, and both have an IC50 that is <0.51 nM, whereas the positive control Cetrorelix has an IC50 of 0.08 nM. Overall, the data indicated that these novel cell surface receptor ligand analogs gain the ability to form aqueous gel and retain the ability to regulate the receptor activities.

TABLE 4
List of the bioactivity of synthetic gel-forming ligands

| | | | Testing | |
|---|---|---|---|---|
| Receptor identity | | | Compounds | |
| | | Positive | Ligand 1 | Ligand 2 |
| | | Control | | |
| Melanocortin receptor | | Melanotan II | SEQ ID | SEQ ID |
| 1 (MC1R) | | | NO: 224 | NO: 226 |
| Agonistic assay | EC50 (nM) | 0.56 | <0.05 | 0.14 |
| | Max Activity | | | |
| | % of positive control | 102 | 120 | 115 |
| Melanocortin receptor | | Melanotan II | SEQ ID | |
| 4 (MC4R) | | | NO: 228 | |
| Agonistic assay | EC50 (nM) | 2 | 0.15 | |
| | Max Activity | | | |
| | % of positive control | 101 | 115 | |
| CRH receptor 2 | | Sauvagine | SEQ ID | SEQ ID |
| (CRHR2) | | | NO: 230 | NO: 231 |
| Agonistic assay | EC50 (nM) | 3.6 | 0.5 | 5.7 |
| | Max Activity | | | |
| | % of positive control | 103 | 100 | 78 |
| PTH receptor 1 | | PTH (1-34) | SEQ ID | |
| (PTHR1) | | | NO: 232 | |
| Agonistic assay | EC50 (nM) | 0.8 | 14.1 | |
| | Max Activity | | | |
| | % of positive control | 101 | 59 | |
| Kappa opioid | | Dynorphin A | SEQ ID | |
| receptor (OPRK1) | | | NO: 214 | |
| Agonistic assay | EC50 (nM) | 0.5 | 272 | |
| | Max Activity | | | |
| | % of positive control | 101 | 62 | |
| Bradykinin receptor 2 | | HOE140 | SEQ ID | |
| (BDKRB2) | | | NO: 234 | |
| Antagonistic assay | IC50 (nM) | 131.8 | 53.3 | |
| | Max Activity | | | |
| | % of positive control | 100 | 112 | |
| GnRH receptor | | Cetrorelix | SEQ ID | SEQ ID |
| (GnRHR) | | | NO: 202 | NO: 203 |
| Antagonistic assay | IC50 (nM) | 0.08 | <0.51 | <0.51 |
| | Max Activity | | | |
| | % of positive control | 91.4 | 135 | 109 |

To provide an overall picture of the effects of gel-forming-enhancing motifs on the gel-forming activities of various polypeptides, the ability to form semisolid gel or liquid gel of individual peptides is presented in Table 5 for each pair of the conjugated and wild-type peptides. An ability to form semisolid gel is represented by an SS, and the ability to form liquid gel is represented by an LG. On the other hand, the lack of ability to form a gel at selected concentration is represented by a blank. The t-test analysis showed that the conjugation of a gel-forming-enhancing motif to 57 distinct peptides significantly increased the gel-forming capability of these peptides.

TABLE 5
A heatmap of the prospensity to form gels by ligands with a gel-forming-enhancing motif

| Chimeric or mutated polypeptides | | | | | Wild-type Counterparts | | | | |
|---|---|---|---|---|---|---|---|---|---|
| SEQ ID NO | 20% | 11% | 6% | 3% | 20% | 11% | 6% | 3% | SEQ ID NO |
| 201 | | SS | SS | SS | | LG | AS/IS | | 15 |
| 202 | | SS | SS | AS/IS | | AS/IS | | | 27 |
| 204 | | SS | SS | | | AS/IS | | | 17 |
| 205 | | SS | LG | | | SS | AS/IS | | 5 |
| 206 | LG | LG | | | AS/IS | AS/IS | AS/IS | | 18 |
| 207 | LG | LG | | | AS/IS | AS/IS | | | 19 |
| 208 | SS | LG | | | | SS | SS | | 6 |
| 209 | | SS | SS | SS | | AS/IS | AS/IS | | 20 |
| 210 | | SS | SS | | | AS/IS | AS/IS | | 21 |
| 212 | | | | SS | | AS/IS | AS/IS | | 22 |
| 214 | | SS | SS | LG | | SS | SS | SS | 7 |
| 215 | | SS | AS/IS | | LG | AS/IS | | | 23 |
| 216 | LG | LG | | | | AS/IS | | | 24 |
| 217 | | SS | LG | | | AS/IS | | | 28 |
| 218 | | SS | SS | | | SS | AS/IS | | 4 |
| 219 | | SS | LG | | | AS/IS | | | 26 |
| 220 | | SS | LG | | | LG | AS/IS | | 14 |
| 221 | | | SS | LG | | LG | AS/IS | | 14 |
| 222 | | SS | LG | | | LG | AS/IS | | 12 |
| 224 | | SS | | | AS/IS | AS/IS | | | 29 |
| 226 | | SS | | | AS/IS | AS/IS | | | 33 |
| 228 | | SS | SS | SS | | AS/IS | | | 34 |
| 229 | LG | AS/IS | | | AS/IS | AS/IS | | | 30 |
| 230 | SS | AS/IS | | | AS/IS | AS/IS | | | 31 |
| 231 | | SS | SS | LG | | SS | SS | LG | 10 |
| 232 | SS | LG | | | | AS/IS | AS/IS | | 25 |
| 234 | SS | AS/IS | AS/IS | | AS/IS | AS/IS | | | 32 |
| 235 | | SS | SS | SS | | SS | SS | LG | 8 |
| 237 | SS | AS/IS | AS/IS | | AS/IS | AS/IS | | | 35 |
| 238 | SS | SS | | | AS/IS | AS/IS | | | 41 |
| 240 | | SS | AS/IS | | SS | LG | | | 13 |
| 241 | | LG | | | AS/IS | AS/IS | | | 36 |
| 242 | | SS | LG | | | AS/IS | | | 37 |
| 244 | | SS | AS/IS | | | SS | | | 11 |
| 245 | SS | AS/IS | | | AS/IS | AS/IS | | | 42 |
| 246 | | SS | SS | | | AS/IS | | | 39 |
| 247 | | SS | | | | AS/IS | | | 44 |
| 248 | SS | LG | | | | AS/IS | AS/IS | | 45 |
| 249 | | SS | AS/IS | | | AS/IS | | | 46 |
| 250 | | SS | AS/IS | | | AS/IS | | | 47 |
| 253 | | SS | AS/IS | | LG | AS/IS | | | 23 |
| 254 | SS | LG | | | AS/IS | AS/IS | AS/IS | | 69 |
| 255 | | SS | LG | AS/IS | | SS | SS | | 61 |
| 256 | LG | LG | | | AS/IS | AS/IS | AS/IS | | 29 |
| 257 | SS | SS | LG | | AS/IS | AS/IS | | | 62 |
| 260 | | LG | LG | | | LG | AS/IS | | 64 |
| 261 | | SS | LG | AS/IS | | AS/IS | AS/IS | | 65 |
| 262 | LG | LG | LG | AS/IS | AS/IS | AS/IS | AS/IS | | 18 |
| 264 | SS | SS | AS/IS | | AS/IS | AS/IS | | | 30 |
| 265 | LG | AS/IS | | | AS/IS | AS/IS | | | 31 |
| 266 | | SS | | | AS/IS | AS/IS | | | 67 |
| 267 | | SS | SS | | SS | LG | | | 13 |
| 268 | | SS | LG | | SS | LG | | | 13 |
| 269 | SS | SS | LG | | | LG | AS/IS | | 14 |
| 270 | SS | SS | LG | | | LG | AS/IS | | 14 |
| 273 | SS | SS | LG | | AS/IS | AS/IS | AS/IS | | 29 |
| 274 | SS | SS | SS | | | LG | AS/IS | AS/IS | 1 |

Because native and modified gel-forming peptides form gel nanostructure through a progressive polymeric reaction that involves electrostatic interactions among monomers, the amphiphile monomers could associate and dissociate in an equilibrium. Accordingly, the gel-forming monomers could associate with each other even in the absence of observable gel nanostructure. To demonstrate that the self-assemble gels of the present invention could function as a carrier for other therapeutic agents, we performed release assays employing semipermeable chambers such as the Centricon® Centrifugal Filter Units. In these experiments, FITC-labeled therapeutic agents were dissolved in solutions with a low concentration of gel-forming or nongel-forming peptides, and centrifuged to separate the free-moving soluble molecules in the lower camber and those aggregate with the gel nanostructures in the upper chamber. The movement of FITC-labled molecules was quantified by a fluorometer. As shown in Table 6, the mixing of an FITC-conjugated morpholino molecule (a nucleic acid), an FITC-labled GnRH molecule (a peptide), and an FITC-labeled dUTP molecule (a nucleotide) with the gel-forming peptide (SEQ ID NO:119; 1 mg/10 ml) led to a 3- to 40-fold reduction in molecule movement of the when compared with samples that do not contain the gel-forming peptide. This reduction is dose-dependent, and a much less reduction was observed in samples with 1 mg/100 ml of the gel-forming peptide. By contrast, when the FITC-labeled compounds were mixed with a nongel-forming peptide (SEQ ID NO:133) in the solution, no significant reduction of molecule movement was observed when compared to the control samples.

TABLE 6
Interactions of gel-forming peptides and therapeutic agents

| FITC-labled | Carrier | Fluorescent Reading |
|---|---|---|
| biomolecules | identity | (average of duplicates) |
| Morpholino | Control | 18.57 |
| | SEQ ID NO:119 1mg/10ml | 5.56 |
| | SEQ ID NO:119 1mg/100ml | 15.83 |
| | SEQ ID NO:133 1mg/10ml | 22.31 |
| | SEQ ID NO:133 1mg/100ml | 20.25 |
| GnRH | Control | 72.50 |
| | SEQ ID NO:119 1mg/10ml | 9.00 |
| | SEQ ID NO:119 1mg/100ml | 58.37 |
| | SEQ ID NO:133 1mg/10ml | 55.43 |
| | SEQ ID NO:133 1mg/100ml | 70.95 |
| dUTP | Control | 6.72 |
| | SEQ ID NO:119 1mg/10ml | 0.14 |
| | SEQ ID NO:119 1mg/100ml | 2.67 |
| | SEQ ID NO:133 1mg/10ml | 9.22 |
| | SEQ ID NO:133 1mg/100ml | 6.23 |

Similarly, studies of the interaction between gel-forming peptides and FITC-labeled small molecules (e.g., naloxone and dexamethasone) using Centricon® Centrifugal Filter Units showed that the mixing of these molecules with gel-forming peptides (SEQ ID NOS:109 and 119) leads to the retention of FITC-labeled molecules in the upper chamber of the filter (Table 7). In samples with SEQ ID NO:109 peptide, less than 30% of naloxone and dexamethasone molecules passed through the filter when compared to the control samples. Likewise, less than 15% of naloxone and dexamethasone molecules in samples with SEQ ID NO:119 peptide passed through the filter when compared to the control samples. These data clearly suggest that the FITC-labeled molecules could aggregate or encapsulate together with the gel nanostructure made of the gel-forming peptide, and this association precludes them from moving through the filter membranes. These data also imply that the gel-forming peptide solution could be useful for the sustained delivery of therapeutic agents even in the absence of observable gel structure/conformation.

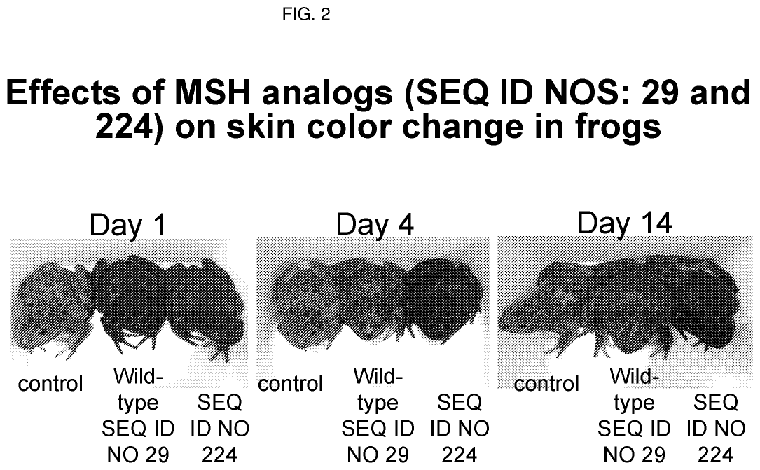

To further demonstrate that the gel-forming peptides have extended biological effects in vivo, we studied the effects of a gel-forming melanocyte-stimulating hormone (MSH) analog (SEQ ID NO: 224) on the skin color change in bull frogs (FIG. 2). Intraperitoneal injection of the wild-type analog (afamelanotide, SEQ ID NO:29; 100 nmoles/kg body weight) or the gel-forming analog (SEQ ID NO: 224, 100 nmoles/kg body weight) to frogs led to a drastic change of skin color. The skin color changed from green to black within 2 hours. The control animals, which received a saline injection, retained the green skin color. At 4 or 14 days after injection, only frogs injected with the gel-forming MSH analog retained the dark skin color resulting from the expansion of melanocytes in the skin, demonstrating the extended effectiveness of the gel-forming analogs.

TABLE 7
Interactions of gel-forming peptides and small molecule drugs

| FITC-labled | Carrier | Fluorescent Reading |
|---|---|---|
| biomolecules | identity | (average of duplicates) |
| Naloxone | Control | 5.43 |
| | SEQ ID NO:109 1mg/10ml | 1.38 |
| | SEQ ID NO:119 1mg/100ml | 0.60 |
| Dexmethasone | Control | 12.93 |
| | SEQ ID NO:109 1mg/10ml | 4.29 |
| | SEQ ID NO:119 1mg/100ml | 1.60 |

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Moreover, due to biological functional equivalency considerations, changes can be made in protein structure without affecting the biological action in kind or amount. All such modifications are intended to be included within the scope of the appended claims.

What is claimed is:

1. An aqueous pharmaceutical composition for sustained release of therapeutic ingredient(s), comprising:
a self assembling gel-forming polypeptide at a concentration of at least about 0.01% by weight relative to the total weight of the composition (w/w); and b) an aqueous excipient.

2. The pharmaceutical composition of claim 1, wherein the gel-forming polypeptide forms a non-covalently linked liquid gel or semisolid gel in aqueous solution at a concentration of from about 11-30% w/w of the solution, or at lower concentrations.

3. The pharmaceutical composition of claim 1, wherein the aqueous excipient is low ionicity.

4. The pharmaceutical composition of any of claims 1-3, further comprising an additive selected from a buffer, an excipient, a solvent, a solubilizer, preservative, stabilizers, surfactants, antioxidants and mixtures thereof.

5. The pharmaceutical composition of any of claims 1-4 wherein the self-assembling gel-forming polypeptide is a G protein-coupled receptor (GPCR) polypeptide ligand or a peptide biological function mediator.

6. The pharmaceutical composition of claim 5, wherein the ligand is selected from amylin, CGRP, adrenomedullin (ADM), and adrenomedullin 2 (ADM2 or IMD) analogs, where the analog may be an agonist, antagonist, chimeric or nonfunctional analog.

7. The pharmaceutical composition of any of claims 1-4, wherein the self assembling gel-forming polypeptide is an analog of oxytocin, kisspeptin, a kappa opioid receptor agonist, Pralmorelin, a Romiplostim analog, urocortin 3, compstatin, GLP-1, GLP-2, thymosin alpha 1, thymosin beta 4, gamma-MSH, a bombesin receptor antagonist, an opioid receptor ligand, or GnRH, where the analog may be an agonist, antagonist, chimeric or nonfunctional analog.

8. The pharmaceutical composition of any of claims 1-7, wherein the gel-forming polypeptides are present in a concentration ranging from 0.001 to 99% by weight, preferably from 0.1 to 30% by weight relative to the total weight of the composition.

9. The pharmaceutical composition of any of claims 1-8, wherein the gel-forming peptide comprises a sequence set forth in any of Tables 1, 2 or 3.

10. The pharmaceutical composition of any of claims 1-8, wherein the gel-forming polypeptide comprises a sequence selected from SEQ ID NOS:1-15, 61, 64, 263, 274, or an analog thereof.

11. The pharmaceutical composition of any of claims 1-4 wherein the gel-forming polypeptide is selected from ADM, CGRP, or IMD (ADM2) sequences comprising a structure of Formula I: R1-B0-B1-B2-B3-B4-B5-B6-B7-B8-B9-B10-B11-B12-B13-B14-B15-B16-B17-B18-B19-B20-B21-B22-B23-B24-B25-B26-B27-B28-R2, where:
R1 a functional group comprising a structure of Formula (W′)(X′)n(Y′)n(Z′)n, wherein W is a fatty acid, a fatty diacid, a fatty acid or cholesterol derivative or empty; X′ is a PEG group, glutamic acid, γ-glutamic acid, a non-proteinogenic amino acid, or empty; Y′ is a PEG group, glutamic acid, γ-glutamic acid, a non-proteinogenic amino acid, or empty; Z′ is a proteinogenic amino acid, a non-proteinogenic amino acid, or empty;
R2 is an C-terminal modification including an {NH2} amidation, {—CHO} peptide aldehydes, {-ol} alcohol peptide, {CMK} chloromethylketone, {FMK} Fluoromethylketone, {Cya} Cysteamide, {pNA} p-nitroaniline, {—ONP} para-nitrophenol, {AMC} 7-Amino-4-methylcoumarin, {AFC}, —OMe (C-terminal), —OEt (C-terminal), —OBzl (C-terminal), —OtBu (C-terminal), {—OSu} hydroxysuccinimide ester, —NHMe (C-terminal), NHEt (C-terminal), —NHisopen (C-terminal), NH(CH2)6 (C-terminal), —NHPh (C-terminal), {NHEt(O)EtNH-Fmoc} 2,2′-Oxydi Ethanamine-Fmoc, {NHEt(EtNH-Myr)2}, —NH(OMe)Me (C-terminal), -TBzl (C-terminal), —NHNH2 (C-terminal), -ED (C-terminal) —NH—CH2CH2-NH2, or -BD (C-terminal) —NH—CH2CH2CH2CH2-NH2NH2 group;
B0 is selected from the group consisting of an empty residue, any proteinogenic amino acid or non-proteinogenic amino acid, acylated histidine (acy-His), acylated arginine (acy-Arg), acylated lysine (acy-Lys);
B1 is selected from the group consisting of an empty residue, Val, Ala, Gly, Ile, Leu, His, Arg, Lys, Asn, Gln and a non-proteinogenic amino acid;
B2 is selected from the group consisting of an empty residue, Arg, Lys, Gln, Glu, Asp, Asn and a non-proteinogenic amino acid;
B3 is selected from the group consisting of an empty residue, Ala, Leu, Ile, Val, Met, Phe, His, Arg, Lys, Gln, Asp and a non-proteinogenic amino acid;
B4 is selected from the group consisting of an empty residue, Val, Ala, Gly, Ile, Leu and a non-proteinogenic amino acid;
B5 is selected from the group consisting of an empty residue, Val, Ala, Gly, Ile, Leu, Pro, Ser, Th, Tyr and a non-proteinogenic amino acid;
B6 is selected from the group consisting of an empty residue, Ala, Leu, Ile, Val, Met, Phe, His, Arg, Lys and a non-proteinogenic amino acid;
B7 is selected from the group consisting of an empty residue, Ala, Leu, Ile, Val, Met, Phe, Gln, Asn, His, Arg, Lys and a non-proteinogenic amino acid;
B8 is selected from the group consisting of an empty residue, Val, Ala, Gly, Ile, Leu, Ser, Thr and a non-proteinogenic amino acid;
B9 is selected from the group consisting of an empty residue, Arg, Lys, Asn, Gln, Trp, Phe, Ser, Thr, Tyr and a non-proteinogenic amino acid;
B10 is selected from the group consisting of an empty residue, Ala, Ser, Thr, Gln, Glu, Asp, Asn and a non-proteinogenic amino acid;
B11 is selected from the group consisting of an empty residue, Trp, Phe, Val, Ala, Gly, Ile, Leu, Pro and a non-proteinogenic amino acid;
B12 is selected from the group consisting of an empty residue, Ala, Gly, Ser, Thr, Pro, Tyr; Met, Trp, Phe and a non-proteinogenic amino acid;
B13 is selected from the group consisting of an empty residue, Gln, Glu, Asp, and Asn, Val, Ala, Gly, Ile, Leu, Met, Phe and a non-proteinogenic amino acid;
B14 is selected from the group consisting of an empty residue, His, Arg, Lys, Val, Ala, Gly, Ile, Leu, Met, Phe, Pro and a non-proteinogenic amino acid;
B15 is selected from the group consisting of an empty residue, Arg, Lys, Gln, Glu, Asp, Asn, Val, Ala, Gly, Ile, Leu and a non-proteinogenic amino acid;
B16 is selected from the group consisting of an empty residue, Asn, Gln, Val, Ala, Gly, Ile, Leu and a non-proteinogenic amino acid;
B17 is selected from the group consisting of an empty residue, Asn, Gln, Ser, Thr, Tyr and a non-proteinogenic amino acid;
B18 is selected from the group consisting of an empty residue, Val, Ala, Gly, Ile, Leu, Phe, Tyr and a non-proteinogenic amino acid;
B19 is selected from the group consisting of an empty residue, Val, Ala, Gly, Ile, Leu, Met, Phe, Pro and a non-proteinogenic amino acid;
B20 is selected from the group consisting of an empty residue, His, Arg, Lys, Val, Ala, Gly, Ile, Leu, Pro and a non-proteinogenic amino acid;
B21 is selected from the group consisting of an empty residue, Ile, Val, Ser, Thr, Tyr, Gln, Glu, Asp, Asn and a non-proteinogenic amino acid;
B22 is selected from the group consisting of an empty residue, His, Arg, Lys, Val, Ala, Gly, Ile, Leu, Asn, Gln, Pro and a non-proteinogenic amino acid;
B23 is selected from the group consisting of an empty residue, Ser, Thr, Tyr, Val, Ala, Gly, Ile, Leu, Met, Phe and a non-proteinogenic amino acid;
B24 is selected from the group consisting of an empty residue, Ala, Gly, Pro, Ser, Thr, Tyr and a non-proteinogenic amino acid;
B25 is selected from the group consisting of an empty residue, Val, Ala, Gly, Ile, Leu, Pro, Ser, Thr and a non-proteinogenic amino acid;
B26 is selected from the group consisting of an empty residue, His, Arg, Lys, Gln, Glu, Asp, Asn and a non-proteinogenic amino acid;
B27 is selected from the group consisting of an empty residue, Val, Ala, Gly, Ile, Leu, Ser, Thr, Tyr and a non-proteinogenic amino acid;
B28 is selected from the group consisting of an empty residue, Ala, Leu, Ile, Val, Phe, Ser, Thr, Tyr and a non-proteinogenic amino acid.

12. A functional self-assembling gel-forming polypeptide agonist or antagonist comprising a sequence set forth in SEQ ID NOS:1-15, 48-58, 61, 64, 263, 274, or an analogs thereof.

13. A gel-forming polypeptide, comprising an amino acid sequence having at least 70% sequence identity to an amino acid sequence selected from SEQ ID NOS: 1-15, 48-58, 61, 64, 263, and 274.

14. A gel-forming polypeptide, comprising an amino acid sequence having at least 80% sequence identity to an amino acid sequence selected from SEQ ID NOS: 1-15, 48-58, 61, 64, 263, and 274.

15. A gel-forming polypeptide comprising a sequence selected from SEQ ID NOS: 106-114, 116-124, 126-131, and 139-140, and their analogs thereof.

16. A gel-forming polypeptide, comprising an amino acid sequence having at least 70% sequence identity to an amino acid sequence selected from SEQ ID NOS: 106-114, 116-124, 126-131, and 139-140.

17. A gel-forming polypeptide, comprising an amino acid sequence having at least 80% sequence identity to an amino acid sequence selected from SEQ ID NOS: 106-114, 116-124, 126-131, and 139-140.

18. A gel-forming polypeptide, comprising a stereoisomer, derivative, analogs, or peptidomimetics of an amino acid sequence selected from SEQ ID NOS: 1-15, 48-58, 61, 64, 263, 274, 106-114, 116-124, 126-131, and 139-140.

19. A method for generating a self-assembling liquid or semisolid gel, the method comprising:
dissolving a self assembling gel-forming polypeptide at a concentration of at least about 0.01% by weight relative to the total weight of the composition (w/w); in an aqueous excipient.

20. The method of claim 19, wherein the polypeptide comprises or consists of a sequence set forth in any of SEQ ID NOS: 1-15, 48-58, 61, 64, 263, 274, 106-114, 116-124, 126-131, and 139-140, or an analog thereof.

21. A method for treating a subject suffering from a condition which may be alleviated by administration of a gel-forming polypeptide formulation, the method comprising:
administering to said subject an effective amount of the formulation according to any of claims 1-20.

22. A liquid, semisolid, or solid gel pharmaceutical composition comprising one or more therapeutic agents; a water-soluble gel-forming polypeptide, and optionally an excipient and/or a therapeutic agent, which when injected into a patient forms a gel upon contact with the body.

23. The pharmaceutical composition of claim 22, wherein the therapeutic agent is not covalently linked to a gel-forming polypeptide.

24. The pharmaceutical composition of claim 22 or 23, wherein the gel-forming polypeptide is a GPCR ligand-derived polypeptide or a peptide biological function mediator.

25. The pharmaceutical composition of claim 24, wherein the gel-forming polypeptide comprises or consists of a gel-forming GPCR ligand or ligand fragment selected from SEQ ID NOS: 1-15, 48-58, 61, 64, 263, 274, 106-114, 116-124, 126-131, and 139-140 or an analog thereof.

26. The pharmaceutical composition of any of claims 22-25, wherein the therapeutic agent is selected from small molecule drugs, peptide drugs, large molecule biologicals, antibodies, hormones, growth factors, antigens, nucleic acids, and nucleotides.

27. A method for treating a subject suffering from a condition which may be alleviated by administration of a gel-forming polypeptide formulation, the method comprising:
administering to said subject an effective amount of the formulation according to any of claim 1-11, 12-21, or 22-26, wherein the therapeutic agent is released over an extended period of time.

28. The method of claim 27, wherein the therapeutic agent is released to general circulation, local tissue or organ, or a surface comprising ocular, buccal, rectal, nasal, respiratory, gastrointestinal, urethral, uterine, or skin surfaces.

29. The method of claim 27 or 28, wherein the formulation is administered to the patient parenterally, intramuscularly, subcutaneously, intranasally, intrauterinely, intraurethral, intraocularly, topically, orally, or intradermally.

30. The method of any of claims 27-29, wherein the pharmaceutical formulation is co-administered with one or more other agents, selected from small molecule, polypeptide, proteins, enzyme, hormone, polynucleotides, nucleoprotein, polysaccharide, glycoprotein, lipoprotein, steroids, analgesics, local anesthetics, antibiotic, chemotherapeutic, immunosuppressive, anti-inflammatory, antiproliferative, antimitotic, angiogenic, antiangiogenic, antipsychotic, central nervous system (CNS), anticoagulant, and fibrinolytic drugs; said drugs include nerve growth factor (NGF), brain-derived neurotrophic factor (BDNF), neurotrophin-3 (NT-3), neurotrophin-4/5 (NT-4/5), ciliary neurotrophic factor (CNTF), leukemia inhibitory factor, cardiotrophin-1, basic fibroblast growth factor (bFGF), acidic fibroblast growth factor (aFGF), transforming growth factors β1 (TGFβ1; TGFβ2, TGFβ3), activin, glial cell-derived neurotrophic factor (GDNF), midkine, heparin-binding neurotrophic factor (HBNF), transforming growth factor α (TGFα), heregulin (neuregulin, ARIA), axon ligand-1 (AI-1), epidermal growth factors (EGFs), platelet-derived growth factor (PDGFs), insulin-like growth factors (IGFs), fibroblast-growth factors (FGFs), transforming-growth factors (TGFs), interleukins (ILs), colony-stimulating factors (CSFs, MCFs, GCSFs, GMCSFs), interferons (IFNs), endothelial growth factors (VEGF, EGFs), erythropoietins (EPOs), angiopoietins (ANGs), placenta-derived growth factors (PIGFs), bone morphogenetic proteins (BMPs), growth and differentiation factors (GDFs); antibodies, antigens, adenosines, adrenergic amines, acetycholine, histamine derivative, dopamine derivatives, glutamic acid derivative, GABA derivatives, cannabinoid derivatives, prostanoid derivatives, leukotrienes, thrombin analogs, lysophospholipid (LPA) derivatives, sphingosine 1-phosphate derivatives, LHRH analogs, LHRH antagonist analogs, vasopressin analogs, oxytocin analogs, apelin analogs, neurotensin analogs, kisspeptin analogs, kisspeptin 234 analogs, bombesin analogs, bradykinin analogs, bradykinin antagonist analogs, opioid analogs, deltorphin analogs, encephalin analogs, substance P analogs, angiotensin II analogs, parathyroid hormone analogs, PTHrP analogs, GLP-1 analogs, GLP-2 analogs, glucagon analogs, GIP analogs, calcitonin analogs, amylin analogs, CGRP analogs, adrenomedullin analogs, adrenomedullin 2 analogs, neuropeptide Y (NPY) analogs, peptide YY (PYY) analogs, NPY antagonist analogs, vasoactive intestinal polypeptide (VIP) analogs, urocortin analogs, urocortin 2 analogs, urocortin 3 analogs, bradykinin analogs, somatostatin analogs, endothelin analogs, adrenocorticotropic hormone (ACTH) analogs, melanotan I analogs, melanotan II analogs, melanocyte stimulating hormone (MSH) analogs, melanocortin analogs, growth hormone-releasing hormone analogs, ghrelin analogs, HOE140 analogs, Etelcalcetide analogs, human growth hormone analogs, insulin analogs, antiflammin 1 analogs, thrombin activator analogs, neuromedin U analogs, neuromedin S analogs, heparin, interleukin-1 analogs, interleukin-2 analogs, Factor V analogs, Factor IX analogs, luteinizing hormone analogs, relaxin analogs, ghrelin analogs, follicle-stimulating hormone analogs, atrial natriuretic peptide (ANP) analogs, brain natriuretic peptide (BNP) analogs, C-type natriuretic peptide (CNP) analogs, guanylin analogs, chemokines analogs, cytokine analogs, interferon analogs, erythropoietin analogs, thrombopoietin analogs, interleukin analogs, tumour necrosis factor (TN F) analogs; analogs of thrombopoietin peptide, glatiramer peptide (copaxone), thymosin alpha-1 analogs, thymosin beta 4 analogs, cell-penetrating peptides, TAT peptide, kallikrein inhibitors, phospholipase inhibitors, compstatins, antimicrobial temporin A peptide, antimicrobial Gramicidin peptides, BMP7-derived bone-forming peptide-1, BMP7-derived bone-forming peptide-2, PEDF(24-57), PEDF(58-101), PEDF(40-57), PEDF(44-77), PEDF(78-121), PEDF(98-114), PEDF-derived P14, PEDF-derived P17, PEDF-derived P18, PEDF-derived P23, PEDF-derived P34, PEDF-derived P44, FGF-derived FK18 peptide, Enfuviritide/Fuzeon peptide, Integrilin platelet aggregation inhibitors, YIGSR peptide, RGD peptide, VGVAPG peptide, EEMQRR peptide, and YRSRKYSSWY peptide; toxins such as botulinum toxin and pharmaceutically acceptable salts of these compounds, or their analogs, fragments or derivatives; the salts of the following substances or analogs of: a ligand for adenosine receptors, adrenergic receptors, acetycholine receptors, histamine receptors, dopamine receptors, calcium receptors, glutamic acid receptors, GABA receptors, cannabinoid receptors, prostanoid receptors, leukotriene receptors, proteinase-activated receptor, lysophospholipid (LPA) receptors, sphingosine 1-phosphate receptors, LHRH receptor, vasopressin receptors, oxytocin receptors, apelin receptor, neurotensin receptors, kisspeptin receptor, bombesin receptors, opioid receptors, substance P receptors, angiotensin II receptors, parathyroid hormone receptors, GLP-1 receptor, GLP-2 receptor, glucagon receptor, calcitonin receptor, amylin receptors, calcitonin gene related peptide (CGRP) receptors, adrenomedullin receptors, neuropeptide Y (NPY) receptor, peptide YY (PYY) receptor, vasoactive intestinal polypeptide (VIP) receptor, urocortin receptors, bradykinin receptors, somatostatin receptors, endothelin receptors, adrenocorticotropic hormone (ACTH) receptors, melanocyte stimulating hormone (MSH) receptors, melanocortin receptors, growth hormone releasing hormone receptors, ghrelin receptors, insulin receptors, relaxin receptors, natriuretic peptide receptors, guanylin receptors, chemokine receptors, cytokine receptors, growth factor receptors, interferon receptors, erythropoietin receptors, growth hormone receptor, FSH receptor, LH receptor, TSH receptor, interleukin receptors, tumour necrosis factor (TNF) receptors, nerve growth factor receptors, platelet derived growth factor (PDGF) receptors, colony stimulating factor (CSF) receptors, bone morphogenetic protein (BMP) receptors, FGF receptors, growth and differentiation factor receptors; analogs of glatiramer peptide (copaxone), thymosins, compstatin, temporin A, YIGSR peptide, RGD peptide, VGVAPG peptide, YRSRKYSSWY peptide, as well as nucleotide derivatives, antibiotics, antibodies, enzyme inhibitors, enzymes, complement factors, urokinase, asparaginase, kallikreins, kallikrein inhibitors, and blood clotting factors, cytotoxic therapeutics, microbial antigens, viral antigens, tumor antigens, neoantigens, and cosmetheutical peptides and pharmaceutically acceptable salts of these compounds, or their analogs, fragments or derivatives thereof.

31. A composition according to any of claim 1-11, 12-21, or 22-26, wherein the composition is a pharmaceutical composition, cosmetic composition or a dermal filler composition.

32. A method of engineering a gel-forming polypeptide, the method comprising:
conjugating a therapeutic agent to a gel-forming-enhancing motif; wherein the motif is an acylated or nonacylated amino acid sequence derived from a secreted polypeptide cell surface receptor ligand capable of self-assembling gel formation.

33. The method of claim 32, wherein the gel-enhancing motif is derived from the amino acid sequence of any of SEQ ID NOS: 1-15, 61, 64, 263, 274, 106-114, 116-124, 126-131, or 139-140.

34. The method of claim 32 or 33, wherein the gel-forming polypeptide comprises a structure of Formula II,
Ea-(Fa)n-Ga (II)
wherein Ea is a gel-forming cell-surface-receptor-ligand-derived polypeptide motif or a therapeutic agent; Fa is a PEG group; n is an integral number from 0 to 40; and Ga is a therapeutic agent, or a gel-forming cell-surface-receptor-ligand-derived polypeptide motif. The linkages between Ea, Fa, and Ga can be positioned and bonded through any side chain of the amino acids.

35. An engineered gel-forming polypeptide of formula II
Ea-(Fa)n-Ga (II)
wherein: Ea is a gel-forming polypeptide with the structure of Formula I or a therapeutic agent; Fa is a PEG group; n is an integral number from 0 to 40 or a covalent bond between a motif in Ea and a motif in Ga; and Ga is a therapeutic agent, or a gel-forming polypeptide comprising the structure of Formula I. The linkages between Ea, Fa, and Ga can be positioned and bonded through any side chain of the amino acids.

36. The gel-forming polypeptide of claim 34 or 35, wherein the therapeutic agent(s) are selected from a small molecule, polypeptide, proteins, enzyme, hormone, polynucleotides, nucleoprotein, polysaccharide, glycoprotein, lipoprotein, steroids, analgesics, local anesthetics, antibiotic, chemotherapeutic, immunosuppressive, anti-inflammatory, antiproliferative, antimitotic, angiogenic, antiangiogenic, antipsychotic, central nervous system (CNS), anticoagulant, or fibrinolytic drugs

37. The gel-forming polypeptide of claim 36, wherein the therapeutic agent is selected from: LHRH analogs, LHRH antagonist analogs, vasopressin analogs, oxytocin analogs, apelin analogs, neurotensin analogs, kisspeptin analogs, kisspeptin 234 analogs, bombesin analogs, bradykinin analogs, bradykinin antagonist analogs, opioid analogs, deltorphin analogs, encephalin analogs, substance P analogs, angiotensin II analogs, parathyroid hormone analogs, PTHrP analogs, GLP-1 analogs, GLP-2 analogs, glucagon analogs, GIP analogs, calcitonin analogs, amylin analogs, CGRP analogs, adrenomedullin analogs, adrenomedullin 2 analogs, neuropeptide Y (NPY) analogs, peptide YY (PYY) analogs, NPY antagonist analogs, vasoactive intestinal polypeptide (VIP) analogs, urocortin analogs, urocortin 2 analogs, urocortin 3 analogs, bradykinin analogs, somatostatin analogs, endothelin analogs, adrenocorticotropic hormone (ACTH) analogs, melanotan I analogs, melanotan II analogs, melanocyte stimulating hormone (MSH) analogs, melanocortin analogs, growth hormone-releasing hormone analogs, ghrelin analogs, HOE140 analogs, Etelcalcetide analogs, human growth hormone analogs, insulin analogs, antiflammin 1 analogs, thrombin activator analogs, neuromedin U analogs, neuromedin S analogs, heparin, interleukin-1 analogs, interleukin-2 analogs, Factor V analogs, Factor IX analogs, luteinizing hormone analogs, relaxin analogs, ghrelin analogs, follicle-stimulating hormone analogs, atrial natriuretic peptide (ANP) analogs, brain natriuretic peptide (BNP) analogs, C-type natriuretic peptide (CNP) analogs, guanylin analogs, chemokines analogs, cytokine analogs, interferon analogs, erythropoietin analogs, thrombopoietin analogs, interleukin analogs, tumour necrosis factor (TNF) analogs; analogs of thrombopoietin peptide, glatiramer peptide (copaxone), thymosin alpha-1 analogs, thymosins, cell-penetrating peptides, TAT peptide, kallikrein inhibitors, phospholipase inhibitors, compstatins, antimicrobial temporin A peptide, antimicrobial Gramicidin peptides, BMP7-derived bone-forming peptide-1, BMP7-derived bone-forming peptide-2, PEDF(24-57), PEDF(58-101), PEDF(40-57), PEDF(44-77), PEDF(78-121), PEDF(98-114), PEDF-derived P14, PEDF-derived P17, PEDF-derived P18, PEDF-derived P23, PEDF-derived P34, PEDF-derived P44, FGF-derived FK18 peptide, Enfuviritide/Fuzeon peptide, Integrilin platelet aggregation inhibitors, YIGSR peptide, KTTKS peptide, RGD peptide, VGVAPG peptide, EEMQRR peptide, and YRSRKYSSVVY peptide; toxins such as botulinum toxin, nerve growth factor (NGF), brain-derived neurotrophic factor (BDNF), neurotrophin-3 (NT-3), neurotrophin-4/5 (NT-4/5), ciliary neurotrophic factor (CNTF), leukemia inhibitory factor, cardiotrophin-1, basic fibroblast growth factor (bFGF), acidic fibroblast growth factor (aFGF), transforming growth factors β1 (TGFβ1; TGFβ2, TGFβ3), activin, glial cell-derived neurotrophic factor (GDNF), midkine, heparin-binding neurotrophic factor (HBNF), transforming growth factor α (TGFα), heregulin (neuregulin, ARIA), axon ligand-1 (AI-1), epidermal growth factors (EGFs), platelet-derived growth factor (PDGFs), insulin-like growth factors (IGFs), fibroblast-growth factors (FGFs), transforming-growth factors (TGFs), interleukins (ILs), colony-stimulating factors (CSFs, MCFs, GCSFs, GMCSFs), interferons (IFNs), endothelial growth factors (VEGF, EGFs), erythropoietins (EPOs), angiopoietins (ANGs), placenta-derived growth factors (PIGFs), bone morphogenetic proteins (BMPs), growth and differentiation factors (GDFs); antibodies, antigens, or their analogs, fragments or derivatives; the salts of the following substances or analogs of: a ligand for adenosine receptors, adrenergic receptors, acetycholine receptors, histamine receptors, dopamine receptors, calcium receptors, glutamic acid receptors, GABA receptors, cannabinoid receptors, prostanoid receptors, leukotriene receptors, proteinase-activated receptor, lysophospholipid (LPA) receptors, sphingosine 1-phosphate receptors, LHRH receptor, vasopressin receptors, oxytocin receptors, apelin receptor, neurotensin receptors, kisspeptin receptor, bombesin receptors, opioid receptors, substance P receptors, angiotensin II receptors, parathyroid hormone receptors, GLP-1 receptor, GLP-2 receptor, glucagon receptor, calcitonin receptor, amylin receptors, calcitonin gene related peptide (CGRP) receptors, adrenomedullin receptors, neuropeptide Y (NPY) receptor, peptide YY (PYY) receptor, vasoactive intestinal polypeptide (VIP) receptor, urocortin receptors, bradykinin receptors, somatostatin receptors, endothelin receptors, adrenocorticotropic hormone (ACTH) receptors, melanocyte stimulating hormone (MSH) receptors, melanocortin receptors, growth hormone releasing hormone receptors, ghrelin receptors, insulin receptors, relaxin receptors, natriuretic peptide receptors, guanylin receptors, chemokine receptors, cytokine receptors, growth factor receptors, interferon receptors, erythropoietin receptors, growth hormone receptor, FSH receptor, LH receptor, TSH receptor, interleukin receptors, tumour necrosis factor (TNF) receptors, nerve growth factor receptors, platelet derived growth factor (PDGF) receptors, colony stimulating factor (CSF) receptors, bone morphogenetic protein (BMP) receptors, FGF receptors, growth and differentiation factor receptors; analogs of glatiramer peptide (copaxone), thymosins, compstatin, temporin A, YIGSR peptide, KTTKS peptide, RGD peptide, VGVAPG peptide, YRSRKYSSVVY peptide, nucleotide derivatives, antibiotics, antibodies, enzyme inhibitors, enzymes, complement factors, urokinase, asparaginase, kallikreins, kallikrein inhibitors, and blood clotting factors, cytotoxic therapeutics, microbial antigens, viral antigens, tumor antigens, neoantigens, and cosmetheutical peptides and pharmaceutically acceptable salts of these compounds, or their analogs, fragments or derivatives thereof.

38. The gel-forming polypeptide of any of claims 34-37, wherein the gel-forming component Ea and Ga are selected from a ligand for adenosine receptors, adrenergic receptors, acetycholine receptors, histamine receptors, dopamine receptors, calcium receptors, glutamic acid receptors, GABA receptors, cannabinoid receptors, prostanoid receptors, leukotriene receptors, proteinase-activated receptor, lysophospholipid (LPA) receptors, sphingosine 1-phosphate receptors, LHRH receptor, vasopressin receptors, oxytocin receptors, apelin receptor, neurotensin receptors, kisspeptin receptor, bombesin receptors, opioid receptors, substance P receptors, angiotensin II receptors, parathyroid hormone receptors, GLP-1 receptor, GLP-2 receptor, glucagon receptor, calcitonin receptor, amylin receptors, calcitonin gene related peptide (CGRP) receptors, adrenomedullin receptors, neuropeptide Y (NPY) receptor, peptide YY (PYY) receptor, vasoactive intestinal polypeptide (VIP) receptor, urocortin receptors, bradykinin receptors, somatostatin receptors, endothelin receptors, adrenocorticotropic hormone (ACTH) receptors, melanocyte stimulating hormone (MSH) receptors, melanocortin receptors, growth hormone releasing hormone receptors, ghrelin receptors, insulin receptors, relaxin receptors, natriuretic peptide receptors, guanylin receptors, chemokine receptors, cytokine receptors, growth factor receptors, interferon receptors, erythropoietin receptors, growth hormone receptor, FSH receptor, LH receptor, TSH receptor, interleukin receptors, tumour necrosis factor (TNF) receptors, nerve growth factor receptors, platelet derived growth factor (PDGF) receptors, colony stimulating factor (CSF) receptors, bone morphogenetic protein (BMP) receptors, growth and differentiation factor receptors and pharmaceutically acceptable salts of these compounds, or their analogs, fragments or derivatives thereof.

39. The gel-forming polypeptide of any of claims 34-37, wherein the gel-forming component Ea and Ga are selected from SEQ ID NOS: 1-15, 48-58, 61, 64, 263, 274, 106-114, 116-124, 126-131, and 139-140 or a pharmaceutically acceptable salt thereof.

40. The gel-forming polypeptide of any of claims 34-37 wherein Ea and Ga comprise a stereoisomer, derivative, analogs, or peptidomimetics of an amino acid sequence selected from SEQ ID NOS: 1-15, 48-58, 61, 64, 263, 274, 106-114, 116-124, 126-131, and 139-140.

41. The gel-forming polypeptide of any of claims 34-37 wherein Ea or Ga is a gel-forming polypeptide comprising an amino acid sequence having at least 70% sequence identity to an amino acid sequence selected from SEQ ID NOS: 1-15, 48-58, 61, 64, 263, 274, 106-114, 116-124, 126-131, and 139-140.

42. The gel-forming polypeptide of any of claims 34-37 wherein Ea or Ga is a gel-forming polypeptide comprising an amino acid sequence having at least 80% sequence identity to an amino acid sequence selected from SEQ ID NOS: 1-15, 48-58, 61, 64, 263, 274, 106-114, 116-124, 126-131, and 139-140.

43. The gel-forming polypeptide of any of claims 34-37, wherein the gel-forming polypeptide comprises an amino acid sequence of SEQ ID NOS: 1-15, 61, 64, 263, 274, 106-114, 116-124, 126-131, or 139-140, or an analog thereof.

44. The gel-forming polypeptide of any of claims 34-37, comprising an amino acid sequence selected from SEQ ID NOS:201-275 or an analogs thereof; wherein the functional component of the gel-forming polypeptide is an analog or derivative of GnRH, GnRH antagonist, vasopressin, oxytocin, apelin, neurotensin, kisspeptin, bombesin, bombesin receptor antagonist, deltorphin, enkephalin, a kappa receptor agonist, substance P, saralasin, calcitonin, pramlintide (amylin analog), exenatide 4, GLP-1, Teduglutide (GLP-2 analog), afamelanotide (melanotan I), melanotan II, gamma-MSH, ACTH1-24, setmelanotide, PYY3-36, urocortin 2, urocortin 3, parathyroid hormone, VIP, bradykinin receptor 1 antagonist, HOE140 (a BKR2 antagonist), sermorelin, atrial natriuretic peptide (ANP), thymosin alpha-1, thymosin beta 4, adrenomedullin, adrenomedullin 2, a TAT cell penetrating-enhancing peptide, a kallikrein inhibitor, antimicrobial temporin A, compstatin, Glatiramer peptide (Copaxone), a matrix modifying peptide 1, a matrix modifying peptide 4, a matrix modifying peptide 7, a matrix modifying peptide 8, and an acetyl hexapeptide-3 matrix modifying peptide and their analogs thereof.

45. The gel-forming polypeptide of any of claims 34-44, wherein the polypeptide has a relative activity of at least 0.01% compared to that of a corresponding wild-type polypeptide ligand/enzyme/enzyme substrate/mediator on at least one cognate receptor or a cellular target.

46. A gel-forming polypeptide having at least 70% sequence identity to an amino acid sequence selected from SEQ ID NOS: 201-275.

47. A gel-forming polypeptide having at least 80% sequence identity to an amino acid sequence selected from SEQ ID NOS: 201-275.

48. A pharmaceutical composition comprising a pharmaceutically acceptable excipient and a gel-forming polypeptide according to any of claims 34-47.

49. A pharmaceutical composition according to any of claim 1-11, 22-26 or 48, wherein the active agent is therapeutic for cardiovascular, pulmonary, gastrointestinal, immunological, oncological, cutaneous, renal, endocrine, ocular, muscoskeletal, or neuronal diseases.

50. A pharmaceutical composition according to any of claim 1-11, 22-26 or 48-49, formulated as a liquid or liquid gel for administration by injection, infusion or topocal application

51. A pharmaceutical composition according to any of claim 1-11, 22-26 or 48-49, formulated to cause slow release of a polypeptide and/or a therapeutic agent in an individual.

52. A method of treating and/or preventing a cardiovascular, pulmonary, gastrointestinal, immunological, oncological, cutaneous, renal, endocrine, ocular, muscoskeletal, or neuronal disease or a condition associated with the aberrant regulation of a cellular process in an individual, the method comprising:
administering to the individual an effective amount of a pharmaceutical composition according to any of claim 1-11, 22-26 or 48-49.

53. A device comprising a gel-forming polypeptide of any one of claims 12-18 or an engineered gel-forming polypeptide of any of claims 35-47, for delivery of therapeutic agent(s) to a subject.

54. A kit comprising a gel-forming polypeptide of any one of claims 12-18 or an engineered gel-forming polypeptide of any of claims 35-47, and optionally further comprising packaging and instructions for use.

55. A device according to claim 53 or a kit according to claim 54, further comprising at least one additional therapeutic agent.

56. A pharmaceutical composition comprising an aqueous solution or an aqueous mixture, a suspension, a liquid gel or semisolid gel, or a solid gel pharmaceutical composition of (a) at least one gel-forming polypeptide compound having an aqueous solubility greater than 0.01 mg/mL at room temperature, which is selected from the group consisting of SEQ ID NOS: 1-15, 48-58, 61, 64, 106-114, 116-124, 126-131, 139-140, and 201-275 and analogs and derivatives thereof.

57. A method of eliciting an agonistic or antagonistic effect from a cell surface or intracellular receptor, an enzyme, or a biological process mediator in a subject in need thereof, comprising:
administering to said subject an effective amount of a pharmaceutical composition according to any of claim 1-11, 22-26 or 48-49.

58. The method of claim 57, wherein the receptor/enzyme/enzyme substrate/mediator is a GnRH receptor, vasopressin receptors, oxytocin receptors, apelin receptor, neurotensin receptors, kisspeptin receptor, bombesin receptors, opioid receptors, substance P receptors, angiotensin receptors, ghrelin receptor, parathyroid hormone receptors, PTHrP receptors, GIP receptor, GLP-1 receptor, GLP-2 receptor, glucagon receptor, calcitonin receptor, amylin receptor, CGRP receptors, Adremoedullin receptors, CGRP receptors, MSH receptors, melanocortin receptors, peptide Y receptors, peptide YY receptors, urocortin receptors, bradykinin receptors, GHRH receptors, ACTH receptors, VIP receptor, thrombin receptor, somatostatin receptors, protease-activated receptors, natriuretic peptide receptors, insulin receptors, relaxin receptors, matrix proteins, cellular targets of metrkine matrix-modifying peptides, thymosins, thymosin alpha-1, thymosin beta 4, kallikrein inhibitors, thrombopoietin receptor binding domain (or Romiplostim analog), matrikines peptides, Glatiramer peptide (Copaxone), antibiotics and antimicrobial agent (e.g., temporin A), complement regulators (e.g., compstatin), a cell-penetrating peptide-containing molecule for intracellular delivery of a compound (e.g., TAT peptide), microbial antigen, viral antigen, neoantigen, tumor antigen, or a cytotoxic agent.

59. A method of eliciting a sustained immune response in a patient or an animal, which comprises administering to said subject an effective amount of a pharmaceutical composition according to any of claim 1-11, 22-26 or 48-49.

60. A method of generating a gel-forming compounds for eliciting a sustained immune response in a patient or an animal, wherein the gel-forming compounds is generated via the conjugation of a gel-forming-enhancing motif to an antigen or neoantigen; said gel-forming-enhancing motif is selected from an amino acid sequence selected from SEQ ID NOS: 1-15, 61, 64, 263, 274, 106-114, 116-124, 126-131, and 139-140, and their analogs or derivatives thereof.

61. A method of delivering a gel-forming polypeptide of any one of claims 12-18 or an engineered gel-forming polypeptide of any of claims 35-47, the method comprising coating the surface of an implantable device or a tissue with a gel derived from the polypeptide.

62. A method of encapsulating a therapeutic agent, an antigen, a nanostructure, organelles, or cells with a gel-forming polypeptide of any one of claims 12-18 or an engineered gel-forming polypeptide of any of claims 35-47, the method comprising holding the therapeutic agent, the antigen, the nanostructure, the organelles, or the cells within a confined space with a gel derived from the polypeptide.
